(19) United States
(12) Patent Application Publication
(10) Pub. No.: US 2025/0000991 A1
(43) Pub. Date: Jan. 2, 2025

(54) NECTIN-4 ANTIBODIES AND ANTIBODY-DRUG CONJUGATES

(71) Applicant: Eli Lilly and Company, Indianapolis, IN (US)

(72) Inventors: Jeffrey Streetman BOYLES, Indianapolis, IN (US); Kyla Elizabeth DRISCOLL, Rutherford, NJ (US); Omar DURAMAD, Berkeley, CA (US); Qianxu GUO, Ossining, NY (US); Rikke Baek HOLMGAARD, West New York, NJ (US); Kevin Charles LINDQUIST, Berkeley, CA (US); Joshua Inshik PARK, Castro Valley, CA (US); Divya SAGAR, Hoboken, NJ (US); Mohan SRINIVASAN, Cupertino, CA (US); Petra VERDINO, San Diego, CA (US); Jieyu ZHOU, Pleasanton, CA (US)

(21) Appl. No.: 18/747,793

(22) Filed: Jun. 19, 2024

Related U.S. Application Data
(60) Provisional application No. 63/509,077, filed on Jun. 20, 2023.

(51) Int. Cl. A61K 47/68; A61K 45/06; A61P 35/00; C07K 16/28

(52) U.S. Cl. CPC A61K 47/68037; A61K 45/06; A61K 47/6849; A61K 47/6889; A61P 35/00; C07K 16/2803; C07K 2317/565

(57) ABSTRACT

The present disclosure provides nectin-4 antibody drug conjugates and pharmaceutical compositions thereof, and methods of using for the treatment of cancer.

### REFERENCE TO A SEQUENCE LISTING

The present application is being filed along with a Sequence Listing in ST.26 XML format. The Sequence Listing is provided as a file titled “30650_US” created 3 May 2024 and is 120 kilobytes in size. The Sequence Listing information in the ST.26 XML format is incorporated herein by reference in its entirety.

### BACKGROUND

The disclosure relates to the field of medicine. More particularly, the disclosure relates to nectin-4 antibody drug conjugates and pharmaceutical compositions thereof, and their use in treating cancer.

Nectin-4 is a member of the nectin family of Ca2+ independent immunoglobulin-like cellular adhesion molecules. Unlike others in the nectin family, nectin-4 expression in healthy tissue is largely placental or embryonic, but is overexpressed in several tumor types, including urothelial, breast, lung, gastric, colorectal, pancreatic, and ovarian cancer. Studies have connected high nectin-4 expression with tumor occurrence in several cancer types.

Antibody drug conjugates (ADCs) for use as oncology treatments contain a tumor-targeting antibody conjugated to a payload designed to be cell-killing once inside the tumor cell. Certain nectin-4 antibodies have been used to create ADCs with an MMAE payload (WO201247724) and with certain camptothecin analogs (WO2022112356 and WO2021151984).

ADCs for use in oncology are very challenging compounds to design since multiple aspects of the molecule must be balanced, including, sufficient specificity for the tumor target over healthy cells, acceptable toxicity while maintaining desirable activity against bystander tumor cells, and labile payloads to allow intracellular delivery yet maintain good physical and chemical stability.

### SUMMARY

There remains a need for nectin-4 ADCs for treating cancer. In particular, nectin-4 ADCs are needed with a sufficient therapeutic index based on better tolerability and/or better efficacy to support high enough doses to effectively kill the tumor cells but also be tolerable for the patients. In particular, a need remains for nectin-4 ADCs with an effector null antibody and a topoisomerase I payload. In particular, a need remains for nectin-4 ADCs with enhanced bystander activity against nectin-4-low tumors while allowing lower dosing. In particular, a need remains for nectin-4 ADCs that avoid or allow for better management of dermatological events seen in certain nectin-4 ADCs. In particular, a need remains for nectin-4 ADCs that avoid ocular and/or peripheral neuropathy signals seen in certain nectin-4 ADCs. In particular, a need remains for nectin-4 ADCs that have low immunogenicity, stable in vivo pharmacokinetics, and adequate chemical and physical stability. Additionally, a need remains for nectin-4 ADCs that possess one or more of the following features: exhibit better anti-tumor activity as measured in certain tumor models, enhanced bystander activity for nectin-4 low tumors, lower immunogenicity, no measurable antibody effector function, and/or better physical and chemical stability. The ADCs provided herein address one or more of these needs.

Provided herein are certain nectin-4 ADCs and compositions comprising a nectin-4 ADC. Also provided herein are methods of using the nectin-4 ADCs or compositions comprising a nectin-4 ADC for cancer in a subject.

In one aspect, provided herein is an antibody that binds human nectin-4, wherein the antibody comprises a heavy chain variable region (HCVR) and a light chain variable region (LCVR), wherein the HCVR comprises heavy chain complementarity determining regions (HCDR) HCDR1, HCDR2, and HCDR3, and the LCVR comprises light chain complementarity determining regions (LCDR) LCDR1, LCDR2, and LCDR3, wherein
a) the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9;
b) the HCDR1 comprises SEQ ID NO: 18, the HCDR2 comprises SEQ ID NO: 19, the HCDR3 comprises SEQ ID NO: 20, the LCDR1 comprises SEQ ID NO: 21, the LCDR2 comprises SEQ ID NO: 22, and the LCDR3 comprises SEQ ID NO: 23;
c) the HCDR1 comprises SEQ ID NO: 28, the HCDR2 comprises SEQ ID NO: 29, the HCDR3 comprises SEQ ID NO: 30, the LCDR1 comprises SEQ ID NO: 31, the LCDR2 comprises SEQ ID NO: 32, and the LCDR3 comprises SEQ ID NO: 33;
d) the HCDR1 comprises SEQ ID NO: 38, the HCDR2 comprises SEQ ID NO: 39, the HCDR3 comprises SEQ ID NO: 40, the LCDR1 comprises SEQ ID NO: 41, the LCDR2 comprises SEQ ID NO: 42, and the LCDR3 comprises SEQ ID NO: 43;
e) the HCDR1 comprises SEQ ID NO: 48, the HCDR2 comprises SEQ ID NO: 49, the HCDR3 comprises SEQ ID NO: 50, the LCDR1 comprises SEQ ID NO: 41, the LCDR2 comprises SEQ ID NO: 42, and the LCDR3 comprises SEQ ID NO: 51;
f) the HCDR1 comprises SEQ ID NO: 56, the HCDR2 comprises SEQ ID NO: 57, the HCDR3 comprises SEQ ID NO: 58, the LCDR1 comprises SEQ ID NO: 59, the LCDR2 comprises SEQ ID NO: 60, and the LCDR3 comprises SEQ ID NO: 61;
g) the HCDR1 comprises SEQ ID NO: 66, the HCDR2 comprises SEQ ID NO: 67, the HCDR3 comprises SEQ ID NO: 68, the LCDR1 comprises SEQ ID NO: 69, the LCDR2 comprises SEQ ID NO: 70, and the LCDR3 comprises SEQ ID NO: 71; or
h) the HCDR1 comprises SEQ ID NO: 76, the HCDR2 comprises SEQ ID NO: 77, the HCDR3 comprises SEQ ID NO: 78, the LCDR1 comprises SEQ ID NO: 69, the LCDR2 comprises SEQ ID NO: 70, and the LCDR3 comprises SEQ ID NO: 79.

In a further aspect, provided herein is an antibody that binds human nectin-4, wherein the antibody comprises a heavy chain variable region (HCVR) and a light chain variable region (LCVR), wherein
a) the HCVR comprises SEQ ID NO: 10 and the LCVR comprises SEQ ID NO: 11;
b) the HCVR comprises SEQ ID NO: 14 and the LCVR comprises SEQ ID NO: 15;
c) the HCVR comprises SEQ ID NO: 24 and the LCVR comprises SEQ ID NO: 25;
d) the HCVR comprises SEQ ID NO: 34 and the LCVR comprises SEQ ID NO: 35;
e) the HCVR comprises SEQ ID NO: 44 and the LCVR comprises SEQ ID NO: 45;
f) the HCVR comprises SEQ ID NO: 52 and the LCVR comprises SEQ ID NO: 53;
g) the HCVR comprises SEQ ID NO: 62 and the LCVR comprises SEQ ID NO: 63;
h) the HCVR comprises SEQ ID NO: 72 and the LCVR comprises SEQ ID NO: 73; or
i) the HCVR comprises SEQ ID NO: 80 and the LCVR comprises SEQ ID NO: 81.

In another aspect, provided herein is an antibody that binds human nectin-4, wherein the antibody comprises a heavy chain (HC) and a light chain (LC), wherein:
a) the HC comprises amino acids 2-444 of SEQ ID NO: 2 and the LC comprises amino acids 2-215 of SEQ ID NO: 3;
b) the HC comprises amino acids 2-444 of SEQ ID NO: 12 and the LC comprises amino acids 2-215 of SEQ ID NO: 13;
c) the HC comprises amino acids 2-443 of SEQ ID NO: 16 and the LC consists of SEQ ID NO: 17;
d) the HC comprises amino acids 2-447 of SEQ ID NO: 26 and the LC consists of SEQ ID NO: 27;
e) the HC comprises amino acids 2-446 of SEQ ID NO: 36 and the LC consists of SEQ ID NO: 37;
f) the HC comprises amino acids 2-446 of SEQ ID NO: 46 and the LC consists of SEQ ID NO: 47;
g) the HC comprises amino acids 2-446 of SEQ ID NO: 54 and the LC consists of SEQ ID NO: 55;
h) the HC comprises amino acids 2-450 of SEQ ID NO: 64 and the LC comprises amino acids 2-216 of SEQ ID NO: 65; or
i) the HC comprises amino acids 2-450 of SEQ ID NO: 74 and the LC comprises amino acids 2-216 of SEQ ID NO: 75.

In another aspect, provided herein is an antibody-drug conjugate (ADC) comprising a nectin-4 antibody disclosed herein conjugated directly or through a linker to a cytotoxic agent.

In a further aspect, provided herein is an ADC wherein the cytotoxic agent is a camptothecin analog comprising the Formula X-Y, wherein:
Y is of Formula

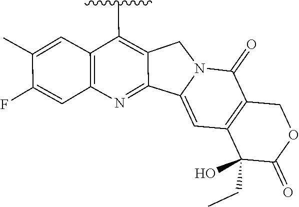 [SMILES: *c1c2c(nc3cc(F)c(C)cc13)-c1cc3c(c(=O)n1C2)COC(=O)[C@]3(O)CC]

and X is *—CH₂O—, *—(CH₂)₂O—, *—(CH₂)₃O—, *—(CH₂)₄O—, *—CH₂NH—, *—(CH₂)₂NH—, *—(CH₂)₃NH—, *—(CH₂)₄NH—, *—CH₂N(CH₃)—, *—(CH₂)₂N(CH₃)—, *—(CH₂)₃N(CH₃)—, *—(CH₂)₄N(CH₃)—, *—CH₂N(R¹)—, *—(CH₂)₂N(R¹)—, *—(CH₂)₃N(R¹)—, *—(CH₂)₄N(R¹)—, *—CH₂N(CH₃)C(═O)CH₂O—, *—CH₂N(CH₂R¹)C(═O)CH₂O—, *—CH₂NHC(═O)CH₂O—, *—CH₂NHC(═O)(CH₂)₂O—, *—CH₂NHC(═O)(CH₂)₃O—, *—CH₂NHC(═O)(CH₂)₄O—, *—CH₂NHC(═O)(CH₂)₅O—, *—CH₂NHC(═O)CH₂—, *—CH₂NHC(═O)(CH₂)₂—, *—CH₂NHC(═O)(CH₂)₃—, *—CH₂NHC(═O)(CH₂)₄—, *—CH₂NHC(═O)(CH₂)₅—, *—CH₂SCH₂—, *—CH₂S(CH₂)₂—, *—CH₂S(CH₂)₃—, *—CH₂S(CH₂)₄—, or *—CH₂S(CH₂)₅—; wherein * is the site covalently attached to Y and R¹ is phenyl.

In another aspect, provided herein is an antibody-drug conjugate (ADC) of the Formula:

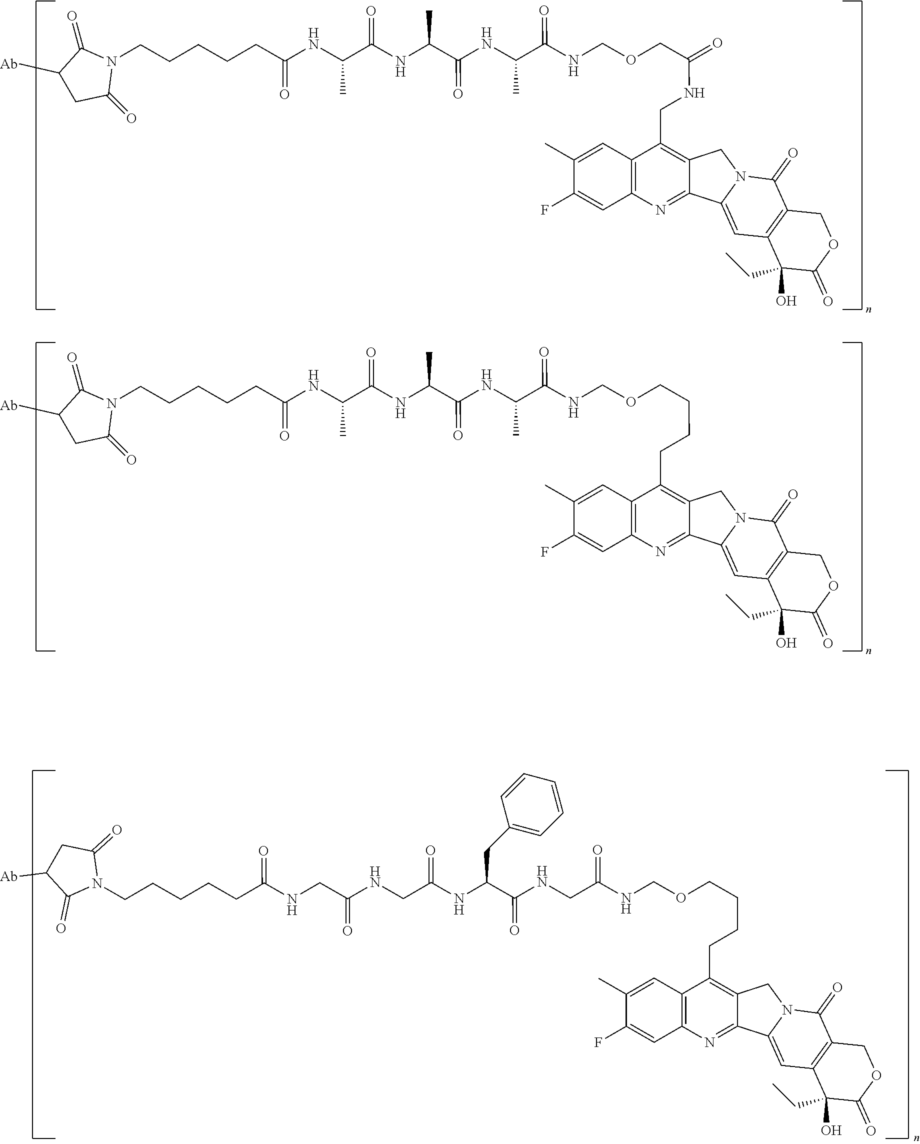

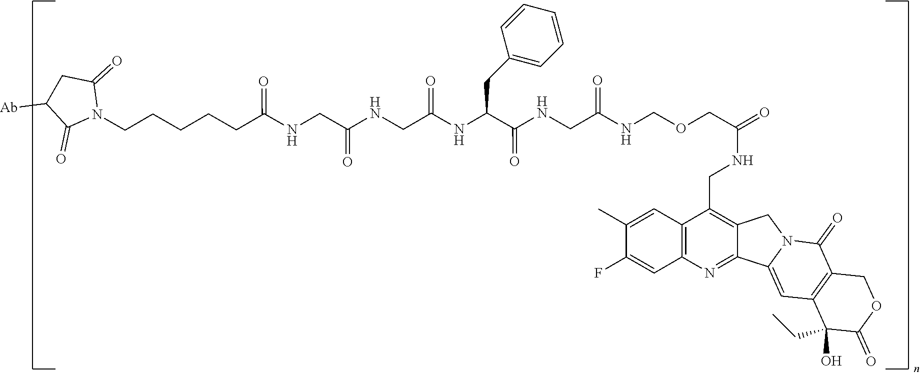

wherein: Ab is a nectin-4 antibody disclosed herein, and n is from about 1 to about 16.

In another aspect, provided herein is a pharmaceutical composition comprising a nectin-4 antibody disclosed herein, and one or more pharmaceutically acceptable carriers, diluents, or excipients. In another aspect, provided herein is a pharmaceutical composition comprising a nectin-4 ADC disclosed herein, and one or more pharmaceutically acceptable carriers, diluents, or excipients.

In another aspect, provided herein is a method of treating cancer, comprising administering to a patient in need thereof, an effective amount of a nectin-4 ADC disclosed herein. In a further aspect, provided herein is a method of treating cancer, comprising administering to a patient in need thereof, an effective amount of a nectin-4 ADC disclosed herein, wherein the cancer is urothelial carcinoma, breast cancer, lung cancer, gastric cancer, colorectal cancer, pancreatic cancer, head and neck cancer, ovarian cancer, or prostate cancer.

### DETAILED DESCRIPTION

### Nectin-4

As used herein, “human nectin-4” refers to a human nectin-4 protein or polypeptide, also known as poliovirus receptor-related 4, Ig superfamily receptor LNIR, or poliovirus receptor-related protein 4 (PVRL4). An amino acid sequence of human nectin-4 can be found at NP_112178.2, including the signal peptide as provided in SEQ ID NO: 1.

### Nectin-4 Antibodies (Also Known as Anti-Nectin-4 Antibodies)

The term “antibody,” as used herein, refers to an immunoglobulin molecule that binds an antigen. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH, also known as HCVR) and a heavy chain constant region. Each light chain is comprised of a light chain variable region (VL, also known as LCVR) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). The CDRs of the present disclosure are determined by North.

Certain antibodies described herein contain an IgG1 Fc region or an Fc region derived from human IgG1, e.g., a modified IgG1 Fc region having altered Fc effector functions. IgG1 is known to induce antibody-dependent cell cytotoxicity (ADCC) and complement-dependent cytotoxicity (CDC). Some antibodies of the present disclosure have amino acid substitutions introduced into IgG1 Fc region altering effector function. According to some disclosed herein, mutations are introduced in the Fc region at positions 234 and 235 (according to the EU Index numbering). According to some disclosed herein, mutations are introduced in the Fc region at positions 234, 235, and 265 (according to the EU Index numbering). In some aspects, the nectin-4 antibodies of the present disclosure comprise a modified human IgG1 Fc region comprising alanine at residues 234 and 235, and serine at position 265 (according to the EU Index numbering, also called hIgG1 effector null or hIgG1EN Fc region). In further aspects, some antibodies have further mutations in the Fc region, including glutamine, alanine, or glycine at position 297, alanine or glutamine at position 322, alanine or glycine at position 329, and/or alanine or serine at position 331 (according to the EU Index numbering). In some aspects, these antibody mutations are alanine at position 234, glutamic acid at position 235, alanine at position 237, serine at position 330, and serine at position 331 (according to the EU Index numbering). In further aspects, these amino acid substitutions introduced into IgG1 Fc region reduced or eliminated measurable antibody effector function.

In certain aspects of the present disclosure, the nectin-4 antibody has a modified human IgG1 or human IgG4 constant domain comprising one or more engineered cysteine residues. In further aspects, the antibody comprises an engineered cysteine in one or more sites within the heavy chain constant domain 1 (CH1), the heavy chain constant domain 2 (CH₂), and/or the heavy chain constant domain 3 (CH₃).

Mammalian expression of antibodies typically results in glycosylation. Glycosylation of antibodies is typically either N-linked or O-linked. N-linked glycosylation refers to the attachment of a carbohydrate moiety to the side chain of an asparagine residue. O-linked glycosylation refers to the attachment of a sugar, for example N-acetylgalactosamine, galactose, or xylose to a hydroxyamino acid. Typically, glycosylation occurs in the Fc region of the antibody at a highly conserved N-glycosylation site (e.g., position 297 in IgG1, according to IMGT or EU Index numbering). Glycosylation sites can be modified to alter glycosylation (e.g., blocking or reducing glycosylation or altering the amino acid sequence to produce additional or diverse glycosylation).

Mammalian expression of antibodies from IgG subclasses can result in clipping of C-terminal amino acids from one or both heavy chains; for example, one or two C-terminal amino acids can be removed for IgG1 antibodies. For IgG1 antibodies, if a C-terminal lysine is present, then it may be truncated or clipped off from the heavy chain during expression. Additionally, a penultimate glycine may also be truncated or clipped off from the heavy chain as well.

Mammalian expression of antibodies can also result in the modification of N-terminal amino acids. For example, where the N-terminal most amino acid of a heavy chain or light chain is a glutamine or glutamic acid, it may be modified into pyro-glutamic acid. For example, where the C-terminal most amino acid of a heavy chain or light chain is a lysine or glycine, it may be removed.

The terms “nucleic acid” or “polynucleotide”, as used interchangeably herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

Polynucleotides of the present disclosure may be expressed in a host cell, for example after the polynucleotides have been operably linked to an expression control sequence. Expression control sequences capable of expression of polynucleotides to which they are operably linked are well known in the art. For example, an expression vector may include a sequence that encodes one or more signal peptides that facilitate secretion of the polypeptide(s) from a host cell. The signal peptide may be an immunoglobulin signal peptide or a heterologous signal peptide, for example. Expression vectors containing a polynucleotide of interest (e.g., a polynucleotide encoding a polypeptide of an antibody) may be transferred into a host cell by well-known methods. Additionally, expression vectors may contain one or more selection markers, e.g., tetracycline, neomycin, and dihydrofolate reductase, to aid in detection of host cells transformed with the desired polynucleotide sequences.

A host cell includes cells stably or transiently transfected, transformed, transduced or infected with one or more expression vectors expressing all or a portion of an antibody of the present disclosure. According to some embodiments, a host cell may be stably or transiently transfected, transformed, transduced, or infected with an expression vector expressing HC polypeptides and an expression vector expressing LC polypeptides of an antibody of the present disclosure. In some embodiments, a host cell may be stably or transiently transfected, transformed, transduced, or infected with an expression vector expressing HC and LC polypeptides of an antibody of the present disclosure. The antibody of the present disclosure may be produced in mammalian cells such as CHO, NS0, HEK293 or COS cells according to techniques well known in the art.

Medium, into which an antibody of the present disclosure has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. The product may be immediately frozen, for example at −70 degrees C., refrigerated, or may be lyophilized. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, NY (1994).

In an aspect, provided herein is an antibody that binds human nectin-4, wherein the antibody comprises a heavy chain variable region (HCVR) and a light chain variable region (LCVR), wherein the HCVR comprises heavy chain complementarity determining regions (HCDR) HCDR1, HCDR2, and HCDR3, and the LCVR comprises light chain complementarity determining regions (LCDR) LCDR1, LCDR2, and LCDR3, wherein the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9. In another aspect, provided herein is an antibody that binds human nectin-4, wherein the HCDR1 comprises SEQ ID NO: 18, the HCDR2 comprises SEQ ID NO: 19, the HCDR3 comprises SEQ ID NO: 20, the LCDR1 comprises SEQ ID NO: 21, the LCDR2 comprises SEQ ID NO: 22, and the LCDR3 comprises SEQ ID NO: 23. In another aspect, provided herein is an antibody that binds human nectin-4, wherein the HCDR1 comprises SEQ ID NO: 28, the HCDR2 comprises SEQ ID NO: 29, the HCDR3 comprises SEQ ID NO: 30, the LCDR1 comprises SEQ ID NO: 31, the LCDR2 comprises SEQ ID NO: 32, and the LCDR3 comprises SEQ ID NO: 33. In another aspect, provided herein is an antibody that binds human nectin-4, wherein the HCDR1 comprises SEQ ID NO: 38, the HCDR2 comprises SEQ ID NO: 39, the HCDR3 comprises SEQ ID NO: 40, the LCDR1 comprises SEQ ID NO: 41, the LCDR2 comprises SEQ ID NO: 42, and the LCDR3 comprises SEQ ID NO: 43. In another aspect, provided herein is an antibody that binds human nectin-4, wherein the HCDR1 comprises SEQ ID NO: 48, the HCDR2 comprises SEQ ID NO: 49, the HCDR3 comprises SEQ ID NO: 50, the LCDR1 comprises SEQ ID NO: 41, the LCDR2 comprises SEQ ID NO: 42, and the LCDR3 comprises SEQ ID NO: 51. In another aspect, provided herein is an antibody that binds human nectin-4, wherein the HCDR1 comprises SEQ ID NO: 56, the HCDR2 comprises SEQ ID NO: 57, the HCDR3 comprises SEQ ID NO: 58, the LCDR1 comprises SEQ ID NO: 59, the LCDR2 comprises SEQ ID NO: 60, and the LCDR3 comprises SEQ ID NO: 61. In another aspect, provided herein is an antibody that binds human nectin-4, wherein the HCDR1 comprises SEQ ID NO: 66, the HCDR2 comprises SEQ ID NO: 67, the HCDR3 comprises SEQ ID NO: 68, the LCDR1 comprises SEQ ID NO: 69, the LCDR2 comprises SEQ ID NO: 70, and the LCDR3 comprises SEQ ID NO: 71. In another aspect, provided herein is an antibody that binds human nectin-4, wherein the HCDR1 comprises SEQ ID NO: 76, the HCDR2 comprises SEQ ID NO: 77, the HCDR3 comprises SEQ ID NO: 78, the LCDR1 comprises SEQ ID NO: 69, the LCDR2 comprises SEQ ID NO: 70, and the LCDR3 comprises SEQ ID NO: 79.

In an aspect, provided herein is an antibody that binds human nectin-4, wherein the antibody comprises a heavy chain variable region (HCVR) and a light chain variable region (LCVR), wherein the antibody comprises a HCVR comprising SEQ ID NO: 10 and a LCVR comprising SEQ ID NO: 11. In another aspect, provided herein is an antibody that binds human nectin-4, wherein the antibody comprises a HCVR comprising SEQ ID NO: 14 and a LCVR comprising SEQ ID NO: 15. In another aspect, provided herein is an antibody that binds human nectin-4, wherein the antibody comprises a HCVR comprising SEQ ID NO: 24 and a LCVR comprising SEQ ID NO: 25. In another aspect, provided herein is an antibody that binds human nectin-4, wherein the antibody comprises a HCVR comprising SEQ ID NO: 34 and a LCVR comprising SEQ ID NO: 35. In another aspect, provided herein is an antibody that binds human nectin-4, wherein the antibody comprises a HCVR comprising SEQ ID NO: 44 and a LCVR comprising SEQ ID NO: 45. In another aspect, provided herein is an antibody that binds human nectin-4, wherein the antibody comprises a HCVR comprising SEQ ID NO: 52 and a LCVR comprising SEQ ID NO: 53. In another aspect, provided herein is an antibody that binds human nectin-4, wherein the antibody comprises a HCVR comprising SEQ ID NO: 62 and a LCVR comprising SEQ ID NO: 63. In another aspect, provided herein is an antibody that binds human nectin-4, wherein the antibody comprises a HCVR comprising SEQ ID NO: 72 and a LCVR comprising SEQ ID NO: 73. In another aspect, provided herein is an antibody that binds human nectin-4, wherein the antibody comprises a HCVR comprising SEQ ID NO: 80 and a LCVR comprising SEQ ID NO: 81.

In a further aspect, provided herein is an antibody that binds human nectin-4, wherein the antibody comprises a heavy chain variable region (HCVR) and a light chain variable region (LCVR), and wherein the antibody has a human IgG1 or IgG4 isotype. In a further aspect, wherein the antibody has a human IgG1 isotype. In a further aspect, wherein the antibody comprises alanine at residues 234 and 235 (according to EU Index numbering). In a further aspect, wherein the antibody further comprises serine at position 265 (according to EU Index numbering). In another aspect, wherein the antibody has a human IgG4 isotype.

In an aspect, provided herein is an antibody that binds human nectin-4, wherein the antibody comprises a heavy chain (HC) and a light chain (LC), wherein the HC comprises amino acids 2-444 of SEQ ID NO: 2 and the LC comprises amino acids 2-215 of SEQ ID NO: 3. In an aspect, provided herein is an antibody that binds human nectin-4, wherein the antibody comprises a heavy chain (HC) and a light chain (LC), wherein the HC comprises amino acids 2-444 of SEQ ID NO: 12 and the LC comprises amino acids 2-215 of SEQ ID NO: 13. In an aspect, provided herein is an antibody that binds human nectin-4, wherein the antibody comprises a heavy chain (HC) and a light chain (LC), wherein the HC comprises amino acids 2-443 of SEQ ID NO: 16 and the LC consists of SEQ ID NO: 17. In an aspect, provided herein is an antibody that binds human nectin-4, wherein the antibody comprises a heavy chain (HC) and a light chain (LC), wherein the HC comprises amino acids 2-447 of SEQ ID NO: 26 and the LC consists of SEQ ID NO: 27. In an aspect, provided herein is an antibody that binds human nectin-4, wherein the antibody comprises a heavy chain (HC) and a light chain (LC), wherein the HC comprises amino acids 2-446 of SEQ ID NO: 36 and the LC consists of SEQ ID NO: 37. In an aspect, provided herein is an antibody that binds human nectin-4, wherein the antibody comprises a heavy chain (HC) and a light chain (LC), wherein the HC comprises amino acids 2-446 of SEQ ID NO: 46 and the LC consists of SEQ ID NO: 47. In an aspect, provided herein is an antibody that binds human nectin-4, wherein the antibody comprises a heavy chain (HC) and a light chain (LC), wherein the HC comprises amino acids 2-446 of SEQ ID NO: 54 and the LC consists of SEQ ID NO: 55. In an aspect, provided herein is an antibody that binds human nectin-4, wherein the antibody comprises a heavy chain (HC) and a light chain (LC), wherein the HC comprises amino acids 2-450 of SEQ ID NO: 64 and the LC comprises amino acids 2-216 of SEQ ID NO: 65. In an aspect, provided herein is an antibody that binds human nectin-4, wherein the antibody comprises a heavy chain (HC) and a light chain (LC), wherein the HC comprises amino acids 2-450 of SEQ ID NO: 74 and the LC comprises amino acids 2-216 of SEQ ID NO: 75.

In a further aspect, provided herein is an antibody that binds human nectin-4, wherein the HC consists of SEQ ID NO: 2 and the LC consists of SEQ ID NO: 3. In another aspect, provided herein is an antibody that binds human nectin-4, wherein the HC consists of SEQ ID NO: 12 and the LC consists of SEQ ID NO: 13. In another aspect, provided herein is an antibody that binds human nectin-4, wherein the HC consists of SEQ ID NO: 16 and the LC consists of SEQ ID NO: 17. In another aspect, provided herein is an antibody that binds human nectin-4, wherein the HC consists of SEQ ID NO: 26 and the LC consists of SEQ ID NO: 27. In another aspect, provided herein is an antibody that binds human nectin-4, wherein the HC consists of SEQ ID NO: 36 and the LC consists of SEQ ID NO: 37. In another aspect, provided herein is an antibody that binds human nectin-4, wherein the HC consists of SEQ ID NO: 46 and the LC consists of SEQ ID NO: 47. In another aspect, provided herein is an antibody that binds human nectin-4, wherein the HC consists of SEQ ID NO: 54 and the LC consists of SEQ ID NO: 55. In another aspect, provided herein is an antibody that binds human nectin-4, wherein the HC consists of SEQ ID NO: 64 and the LC consists of SEQ ID NO: 65. In another aspect, provided herein is an antibody that binds human nectin-4, wherein the HC consists of SEQ ID NO: 74 and the LC consists of SEQ ID NO: 75.

In another aspect, provided herein are different mammalian cells comprising a DNA molecule comprising a polynucleotide sequence encoding polypeptides having the amino acid sequences of SEQ ID NO: 2 and SEQ ID NO: 3, SEQ ID NO: 12 and SEQ ID NO: 13, SEQ ID NO: 16 and SEQ ID NO: 17, SEQ ID NO: 26 and SEQ ID NO: 27, SEQ ID NO: 36 and SEQ ID NO: 37, SEQ ID NO: 46 and SEQ ID NO: 47, SEQ ID NO: 54 and SEQ ID NO: 55, SEQ ID NO: 64 and SEQ ID NO: 65, or SEQ ID NO: 74 and SEQ ID NO: 75, wherein the cell is capable of expressing nectin-4 antibodies disclosed herein.

In another aspect, provided herein are mammalian cells comprising a first DNA molecule and a second DNA molecule, wherein the first DNA molecule comprises a polynucleotide sequence encoding polypeptides having the amino acid sequence as follows, and wherein the second DNA molecule comprises a polynucleotide sequence encoding polypeptides having the amino acid sequence as follows, wherein the cell is capable of expressing nectin-4 antibodies disclosed herein:

| first DNA encodes polypeptide | second DNA encodes polypeptide |
|---|---|
| SEQ ID NO: 2 | SEQ ID NO: 3, |
| SEQ ID NO: 12 | SEQ ID NO: 13, |
| SEQ ID NO: 16 | SEQ ID NO: 17, |
| SEQ ID NO: 26 | SEQ ID NO: 27, |
| SEQ ID NO: 36 | SEQ ID NO: 37, |
| SEQ ID NO: 46 | SEQ ID NO: 47, |
| SEQ ID NO: 54 | SEQ ID NO: 55, |
| SEQ ID NO: 64 | SEQ ID NO: 65, or |
| SEQ ID NO: 74 | SEQ ID NO: 75. |

In another aspect, provided herein is a process for producing a nectin-4 antibody comprising cultivating one of the mammalian cells disclosed herein under conditions such that the antibody is expressed, and recovering the expressed antibody.

In another aspect, provided herein is an antibody produced by cultivating a mammalian cell comprising a DNA molecule comprising a polynucleotide sequence encoding polypeptides having the amino acid sequences of SEQ ID NO: 2 and SEQ ID NO: 3, SEQ ID NO: 12 and SEQ ID NO: 13, SEQ ID NO: 16 and SEQ ID NO: 17, SEQ ID NO: 26 and SEQ ID NO: 27, SEQ ID NO: 36 and SEQ ID NO: 37, SEQ ID NO: 46 and SEQ ID NO: 47, SEQ ID NO: 54 and SEQ ID NO: 55, SEQ ID NO: 64 and SEQ ID NO: 65, or SEQ ID NO: 74 and SEQ ID NO: 75 under conditions such that the antibody is expressed, and recovering the expressed antibody.

In another aspect, provided herein is an antibody produced by cultivating a mammalian cell comprising a first DNA molecule and a second DNA molecule, wherein the first DNA molecule comprises a polynucleotide sequence encoding polypeptides having the amino acid sequence following, and wherein the second DNA molecule comprises a polynucleotide sequence encoding polypeptides having the amino acid sequence following under conditions such that the antibody is expressed, and recovering the expressed antibody:

| first DNA encodes polypeptide | second DNA encodes polypeptide |
|---|---|
| SEQ ID NO: 2 | SEQ ID NO: 3, |
| SEQ ID NO: 12 | SEQ ID NO: 13, |
| SEQ ID NO: 16 | SEQ ID NO: 17, |
| SEQ ID NO: 26 | SEQ ID NO: 27, |
| SEQ ID NO: 36 | SEQ ID NO: 37, |
| SEQ ID NO: 46 | SEQ ID NO: 47, |
| SEQ ID NO: 54 | SEQ ID NO: 55, |
| SEQ ID NO: 64 | SEQ ID NO: 65, or |
| SEQ ID NO: 74 | SEQ ID NO: 75. |

The term “enfortumab” as used herein refers to a fully human anti-nectin-4 IgG1 kappa monoclonal antibody with the sequence as disclosed in FIGS. 3A and 3B of WO2012047724, expressed and purified using standard conditions.

### Payloads

Nectin-4 antibodies of the present disclosure can be conjugated to various payloads (including pharmaceutically acceptable salts thereof) to form an antibody drug conjugate (ADC). Suitable moieties for conjugation to the nectin-4 antibodies disclosed herein include cytotoxic agents (e.g., chemotherapeutic agents), prodrug converting enzymes, radioactive isotopes or compounds, toxins, and other known payloads in the art.

Exemplary ADCs herein utilize camptothecin-based payloads (e.g., a camptothecin analog). Camptothecins are topoisomerase I (TOPO 1) inhibitors that have been shown to have anticancer activity. Camptothecin and its derivatives bind to the TOPO 1/DNA complex which prevents reannealing leading to cell death from the accumulation of partially cleaved DNA. Other topoisomerase I inhibitors are known in the art can be used as payloads, such as SN-38 and DXd.

Other payloads for ADCs described herein are maytansinoids (e.g., DM1 and DM4), pyrrolobenzodiazepines (e.g., PBD dimer), auristatin peptides (e.g., MMAE and MMAF), duocarmycins, calicheamicins, DNA minor groove binders (e.g., enediynes and lexitropsins), and taxanes (e.g., paclitaxel and docetaxel).

In an aspect, provided herein is an ADC, wherein the camptothecin analog comprises the Formula: X-Y, wherein:
Y is of Formula I

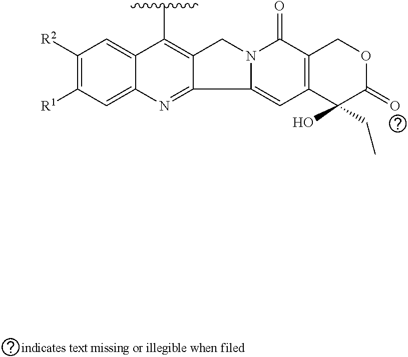

and wherein,
R¹ is F, CH₃, or CF₃;
R² is H, F, OR³, SR³, S(O)R⁴, —S(O)₂R⁴, C₁-C₆ alkyl, or C₁-C₆ fluoroalkyl; or R¹ and R² taken together with the carbon atoms to which they are attached form a methylenedioxy or a difluoromethylenedioxy ring;
R³ is H or C₁-C₆ alkyl;
R⁴ is C₁-C₆ alkyl; and
X is the Formula of A-B
and wherein,
B is absent, —(C₁-C₆ alkylene)-, —(C₁-C₆ alkylene)-X¹—(C₁-C₆ alkylene)-, —X¹′—(C₁-C₆ alkylene)-*, or —(C₁-C₆ alkylene)-X¹-L²-*; where * is the site covalently attached to A;
X¹ is —O—, —S—, —S(O)—, —S(O)₂—, —C(—O)—, —NR⁵—, —NR⁵C(—O)—, or —C(—O)NR⁵—;
each R⁵ is independently —H, C₁-C₆ alkyl, C₁-C₆ fluoroalkyl, C₃-C₆ cycloalkyl, aryl, heteroaryl, or benzyl;
X¹′ is —O—, —S—, —S(O)—, or —S(O)₂—;
L² is phenylene;
A is —H or —X²;
X² is OR⁶, SR⁶, S(O)R⁶, S(O)₂R, SSR⁶, or N(R⁶)₂;
each R⁶ is independently H, C₁-C₆ alkyl, C₁-C₆ fluoroalkyl, C₃-C₆ cycloalkyl, aryl, heteroaryl, or benzyl;
B and L² are each independently optionally substituted with 1-4 substituents selected from halogen, —CN, —OR⁷, —SR⁷, —N(R⁷)₂, C₁-C₆ alkyl, C₁-C₆ fluoroalkyl, C₁-C₆ heteroalkyl, C₃-C₆ cycloalkyl, C₂-C₁₀ heterocycloalkyl, aryl, or heteroaryl; and
each R⁷ is independently H, C₁-C₆ alkyl, C₁-C₆ fluoroalkyl, C₃-C₆ cycloalkyl, aryl, heteroaryl, or benzyl;
with the proviso that if R¹ is F, then B is —(C1-C6 alkylene)-, (C₁-C₆ alkylene)-X¹—(C₁-C₆ alkylene)-, —X¹′—(C₁-C₆ alkylene)-*, or —(C₁-C₆ alkylene)-X¹-L²-*; where * is the site covalently attached to A; and A is —X²; and with the proviso that if R¹ is F and R² is —OMe, then —B-A cannot be —NH₂.

In an aspect, provided herein is an ADC, wherein the camptothecin analog comprises the Formula: X-Y, wherein:
Y is of Formula I

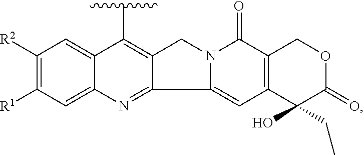

and wherein,
R¹ is F, CH₃, or CF₃;
R² is H, F, OR³, SR³, S(O)R⁴, —S(O)₂R⁴, C₁-C₆ alkyl, or C₁-C₆ fluoroalkyl; or R¹ and R² taken together with the carbon atoms to which they are attached form a methylenedioxy or a difluoromethylenedioxy ring;
R³ is H or C₁-C₆ alkyl;
R⁴ is C₁-C₆ alkyl; and
X is *—CH₂O—, *—(CH₂)₂O—, *—(CH₂)₃O—, *—(CH₂)₄O—, *—CH₂NH—, *—(CH₂)₂NH—, *—(CH₂)₃NH—, *—(CH₂)₄NH—, *—CH₂N(CH₃)—, *—(CH₂)₂N(CH₃)—, *—(CH₂)₃N(CH₃)—, *—(CH₂)₄N(CH₃)—, *—CH₂N(R⁵)—, *—(CH₂)₂N(R⁵)—, *—(CH₂)₃N(R⁵)—, *—(CH₂)₄N(R⁵)—, *—CH₂N(CH₃)C(═O)CH₂O—, *—CH₂N(CH₂R⁵)C(═O)CH₂O—, *—CH₂NHC(═O)CH₂O—, *—CH₂NHC(═O)(CH₂)₂O—, *—CH₂NHC(═O)CH₂)₃O—, *—CH₂NHC(═O)(CH₂)₄O—, *—CH₂NHC(═O)((CH₂)₅O—, *—CH₂NHC(═O)CH₂—, *—CH₂NHC(═O)(CH₂)₂—, *—CH₂NHC(═O)(CH₂)₃—, *—CH₂NHC(═O)(CH₂)₄—, *—CH₂NHC(═O)(CH₂)₅—, *—CH₂SCH₂—, *—CH₂S(CH₂)₂—, *—CH₂S(CH₂)₃—, *—CH₂S(CH₂)₄—, or *—CH₂S(CH₂)₅—; wherein * is the site covalently attached to Y and R⁵ is phenyl.

In an aspect, provided herein is an ADC, wherein the camptothecin analog comprises the Formula: X-Y, wherein:
Y is of Formula II

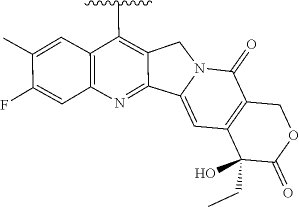 [SMILES: *c1c2c(nc3cc(F)c(C)cc13)-c1cc3c(c(=O)n1C2)COC(=O)[C@]3(O)CC]

and X is *—CH₂O—, *—(CH₂)₂O—, *—(CH₂)₃O—, *—(CH₂)₄O—, *—CH₂NH—, *—(CH₂)₂NH—, *—(CH₂)₃NH—, *—(CH₂)₄NH—, *—CH₂N(CH₃)—, *—(CH₂)₂N(CH₃)—, *—(CH₂)₃N(CH₃)—, *—(CH₂)₄N(CH₃)—, *—CH₂N(R¹)—, *—(CH₂)₂N(R¹)—, *—(CH₂)₃N(R¹)—, *—(CH₂)₄N(R¹)—, *—CH₂N(CH₃)C(═O)CH₂O—, *—CH₂N(R¹)C(═O)CH₂O—, *—CH₂NHC(═O)CH₂O—, *—CH₂NHC(═O)(CH₂)₂O—, *—CH₂NHC(═O)(CH₂)₃O—, *—CH₂NHC(═O)(CH₂)₄O—, *—CH₂NHC(═O)(CH₂)₅O—, *—CH₂NHC(═O)CH₂—, *—CH₂NHC(═O)(CH₂)₂—, *—CH₂NHC(═O)(CH₂)₃—, *—CH₂NHC(═O)(CH₂)₄—, *—CH₂NHC(═O)(CH₂)₅—, *—CH₂SCH₂—, *—CH₂S(CH₂)₂—, *—CH₂S(CH₂)₃—, *—CH₂S(CH₂)₄—, or *—CH₂S(CH₂)₅—; wherein * is the site covalently attached to Y and R¹ is benzyl.

In another aspect, provided herein is an ADC, wherein the camptothecin analog comprises any one of the following Formula:

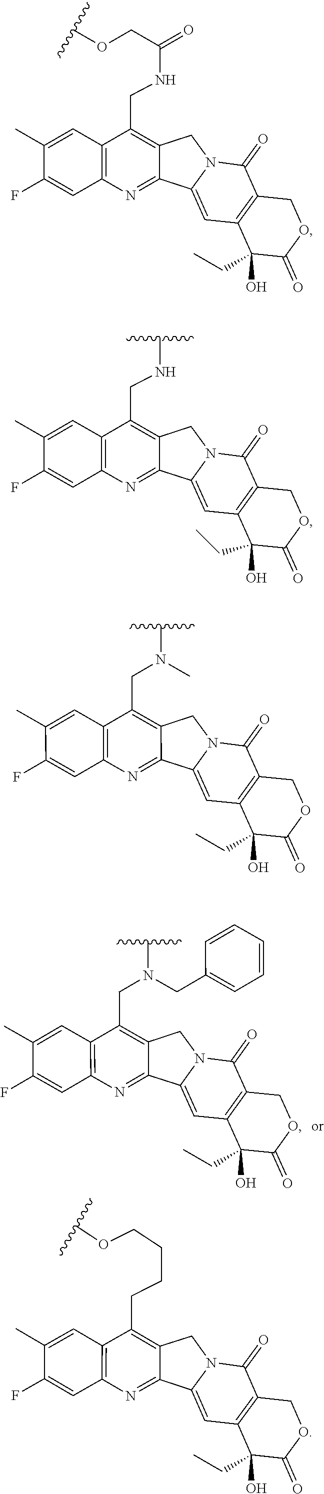 [SMILES: *OCC(=O)NCc1c2c(nc3cc(F)c(C)cc13)-c1cc3c(c(=O)n1C2)COC(=O)[C@]3(O)CC ; *NCc1c2c(nc3cc(F)c(C)cc13)-c1cc3c(c(=O)n1C2)COC(=O)[C@]3(O)CC ; *N(C)Cc1c2c(nc3cc(F)c(C)cc13)-c1cc3c(c(=O)n1C2)COC(=O)[C@]3(O)CC ; *N(Cc1ccccc1)Cc1c2c(nc3cc(F)c(C)cc13)-c1cc3c(c(=O)n1C2)COC(=O)[C@]3(O)CC ; *OCCCCc1c2c(nc3cc(F)c(C)cc13)-c1cc3c(c(=O)n1C2)COC(=O)[C@]3(O)CC]

In a further aspect, provided herein is an ADC, wherein the camptothecin analog comprises the Formula III:

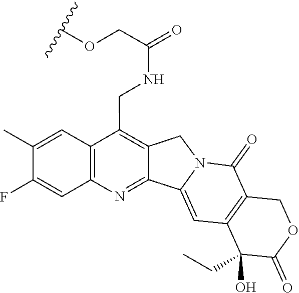 [SMILES: *OCC(=O)NCc1c2c(nc3cc(F)c(C)cc13)-c1cc3c(c(=O)n1C2)COC(=O)[C@]3(O)CC]

In a further aspect, provided herein is an ADC, wherein the camptothecin analog comprises the Formula IV:

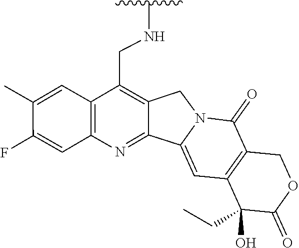 [SMILES: *NCc1c2c(nc3cc(F)c(C)cc13)-c1cc3c(c(=O)n1C2)COC(=O)[C@]3(O)CC]

In a further aspect, provided herein is an ADC, wherein the camptothecin analog comprises the Formula V:

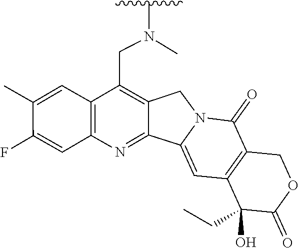 [SMILES: *N(C)Cc1c2c(nc3cc(F)c(C)cc13)-c1cc3c(c(=O)n1C2)COC(=O)[C@]3(O)CC]

In a further aspect, provided herein is an ADC, wherein the camptothecin analog comprises the Formula VI:

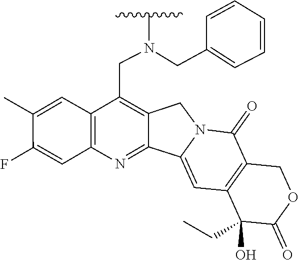 [SMILES: *N(Cc1ccccc1)Cc1c2c(nc3cc(F)c(C)cc13)-c1cc3c(c(=O)n1C2)COC(=O)[C@]3(O)CC]

In a further aspect, provided herein is an ADC, wherein the camptothecin analog comprises the Formula VII:

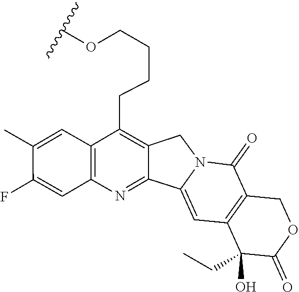 [SMILES: *OCCCCc1c2c(nc3cc(F)c(C)cc13)-c1cc3c(c(=O)n1C2)COC(=O)[C@]3(O)CC]

### Self-Immolative Units

Self-immolation, or self-removal, of a part of the ADC can be designed into the global structure of the ADC. Self-immolation typically involves a trigger group being activated and then chemical and/or biological reactions cause the spontaneous elimination of the group itself, the self-immolation unit. The self-immolative unit can provide positive attributes to the ADC, such as providing space to reduce steric hindrance of cellular proteases reaching the peptide cleavage site in the ADC.

In some aspects of the present disclosure, the ADC described herein contains a self-immolative unit. When present, the self-immolative unit is located on the ADC after the peptide unit of the linker and so the trigger group is exposed after protease cleavage of the peptide unit of the ADC. In further aspects, the self-immolative unit is a —NH—CH₂— group, a para-aminobenzyloxycarbonyl (PABC), ortho-aminobenzyl carbonate (OABC), or other self-immolative units known in the art.

In some aspects of the present disclosure, the ADC described herein does not contain a self-immolative unit. In these aspects, the terminal amine from the camptothecin analog described herein is directly linked to the peptide unit.

### Linkers

As disclosed herein, payloads can be conjugated with a nectin-4 antibody to form a nectin-4 ADC described herein by methods understood by one of skill in the art. One example of such conjugation would include connection of a payload described herein to a nectin-4 antibody described herein via a linker.

Linkers used for ADCs are designed for stability in plasma to allow time for the ADC to localize to the target cells. Releasing the payload too soon lowers the therapeutic index of the ADC by damaging non-targeted tissue of all kinds. When the ADC is internalized into the target cell, then the linker should provide a mechanism for liberation of the payload such so the payload can work as designed.

Linkers known to those of skill in the art contain, for example, cleavable moieties and noncleavable moieties. Accordingly, provided herein are ADCs where the payload, e.g. camptothecin analog, is conjugated to an antibody via a linker with a cleavable moiety or is conjugated to an antibody via a linker with a non-cleavable moiety.

Any suitable linkers known in the art can be used in preparing the ADCs of the present disclosure. In certain aspects, the linkers comprise reactive groups capable of conjugating with both the antibodies of the present disclosure and the drug or cytotoxic agent. Examples include but are not limited to N-succinimidyl-4-(N-maleimidomethyl)-cyclohexane-1-carboxylate (SMCC), V-succinimidyl-4-(iodoacetyl)-aminobenzoate (SIAB), bis-maleimidopolyethyleneglycol (BMPEO), BM(PEO)₂, BM(PEO)₃, N-(b-maleimidopropyloxy)succinimide ester (BMPS), g-maleimidobutyric acid N-succinimidyl ester (GMBS), e-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), k-maleimidoundecanoic acid N-succinimidyl ester (KMUA), N-succinimidyl 4-(p-maleimidophenyl)-butyrate (SMPB), succinimidyl-6-(maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4-bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(y-maleimidobutryloxy)sulfosuccinimide ester (sulfo-GMBS or sGMBS), N-(e-maleimidocaproyloxy)sulfosuccimido ester (sulfo-EMCS), N—(K-maleimidoundecanoyloxy)sulfosuccinimide ester (sulfo-KMUS), and sulfosuccinimidyl 4-(p-maleimidophenyl)butyrate (sulfo-SMPB), succinimidyl 6-hydrazinonicotinamide acetone hydrazone (SANH), succinimidyl 4-hydrazidoterephthalate hydrochloride (SHTH), succinimidyl hydrazinium nicotinate hydrochloride (SHNH), succinimidyl-p-formyl benzoate (SFB), and succinimidyl-p-formylphenoxyacetate (SFPA), V-succinimidyl-3-(2-pyridyldithio)propionate (SPDP), V-succinimidyl-4-(2-pyridyldithio)pentanoate (SPP), N-succinimidyl-4-(2-pyridyldithio)butanoate (SPDB), and V-succinimidyl-4-(2-pyridyldithio)2-sulfo butanoate (sulfo-SPDB).

In certain aspects of the present disclosure, the ADC comprises a cleavable linker. Known in the art are different mechanisms to employ in the linker to release the drug or cytotoxic agent. Included among these mechanisms are (1) utilizing protease cleavage sites in the linker that will be cleaved by cellular proteases (e.g. cathepsin B or beta-glucuronidase, (2) using the lower pH of the lysosome to cause hydrolysis of an acid-labile unit in the linker, or (3) utilizing the higher intracellular levels of glutathione to reduce a disulfide bridge in the linker.

In some aspects of the present disclosure, the ADC comprises a linker which comprises a peptide unit which provides a site for protease cleavage. In some aspects, the peptide unit is -Gly-Gly-Gly-, -Ala-Val-, -Val-Ala-, -Val-Cit-, -Val-Lys-, -Lys-Val-, -Phe-Lys-, -Lys-Phe-, -Lys-Lys-, -Ala-Lys-, -Lys-Ala-, -Phe-Cit-, -Cit-Phe, -Leu-Cit-, -Cit-Leu-, -Ile-Cit-, -Phe-Ala-, -Ala-Phe-, -Phe-Phe-Lys-, -Lys-Phe-Phe-, -Gly-Phe-Lys-, -Lys-Phe-Gly-, -Leu-Ala-Leu-, -Ile-Ala-Leu-, -Leu-Ala-Ile-, -Val-Ala-Val-, -Ala-Leu-Ala-Leu-(SEQ ID NO: 100), -Leu-Ala-Leu-Ala-(SEQ ID NO: 101), -Gly-Phe-Leu-Gly-(SEQ ID NO: 103), -Gly-Leu-Phe-Gly- (SEQ ID NO: 104), -Val-Arg-, -Arg-Val-, -Arg-Arg-, -Ala-Ala-, -Ala-Met-, -Met-Ala-, -Thr-Thr-, -Thr-Met-, -Met-Thr-, -Leu-Ala-, -Ala-Leu-, -Cit-Val-, -Gln-Val-, -Val-Gln-, -Ser-Val-, -Val-Ser-, -Ser-Ala-, -Ser-Gly-, -Ala-Ser-, -Gly-Ser-, -Leu-Gln-, -Gln-Leu-, -Phe-Arg-, -Arg-Phe-, -Tyr-Arg-, -Arg-Tyr-, -Phe-Gln-, -Gln-Phe-, -Val-Thr-, -Thr-Val-, -Met-Tyr-, -Tyr-Met-, -Ala-Ala-, -Ala-Ala-Ala-, -Ala-Ala-Ala-Ala-(SEQ ID NO: 105), -Gly-Ala-Gly-Gly- (SEQ ID NO: 106), -Gly-Gly-Ala-Gly-(SEQ ID NO: 107), -Gly-Val-Gly-Gly- (SEQ ID NO: 108), -Gly-Gly-Val-Gly-(SEQ ID NO: 109), -Gly-Phe-Gly-Gly- (SEQ ID NO: 110), or -Gly-Gly-Phe-Gly- (SEQ ID NO: 102). Citrulline is represented by Cit. In aspects of the disclosure, the peptide unit contains all natural amino acids of the L-amino acid form. In further aspects, the peptide unit may comprise all D-amino acids or L-amino acids, or a combination therein.

In some aspects, provided herein is an ADC further comprising a linker which connects the antibody to the cytotoxic agent. In a further aspect, provided herein the linker comprises a peptide unit. In a further aspect, provided herein the peptide unit comprises Ala-Ala-Ala, Val-Cit, or Gly-Gly-Phe-Gly (SEQ ID NO: 102). In an aspect, the peptide unit comprises Ala-Ala-Ala. In an aspect, the peptide unit comprises Val-Cit. In an aspect, the peptide unit comprises Gly-Gly-Phe-Gly (SEQ ID NO: 102). In a further aspect, the peptide unit contains all natural amino acids of the L-amino acid form.

In some aspects of the present disclosure, the ADC comprises a linker which comprises a spacer unit, herein called spacer unit A, that connects the cysteine(s) of the antibodies disclosed herein to the peptide units and/or payloads described herein. Some of the chemistries used in the art to connect to the cysteine include maleimide, succinimide, or bromoacetamide chemistries, and can be utilized for ADCs of the present disclosure. In further aspects of the present disclosure, maleimide-type spacers such as maleimidocaproyl (mc) or maleimidomethyl cyclohexane-1-carboxylate can be used.

In some aspects of the present disclosure, provided herein is an ADC with a spacer unit A of Formula VIII:

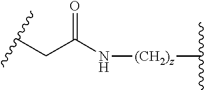

wherein z is from 1-5.

In some aspects of the present disclosure, provided herein is an ADC with a spacer unit A of Formula IX:

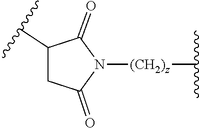

wherein z is from 1-5.

In an aspect of the present disclosure, provided herein is an antibody-drug conjugate (ADC), wherein the ADC is of the Formula:

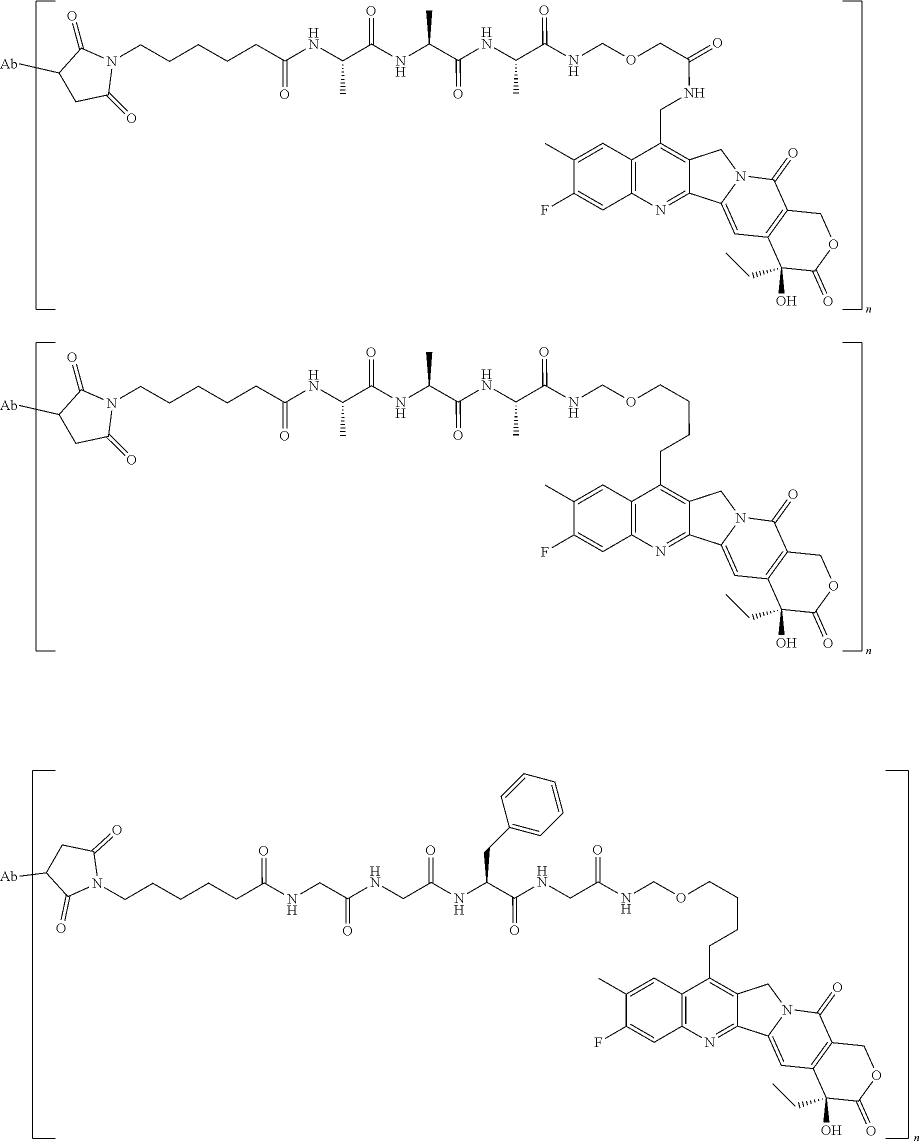

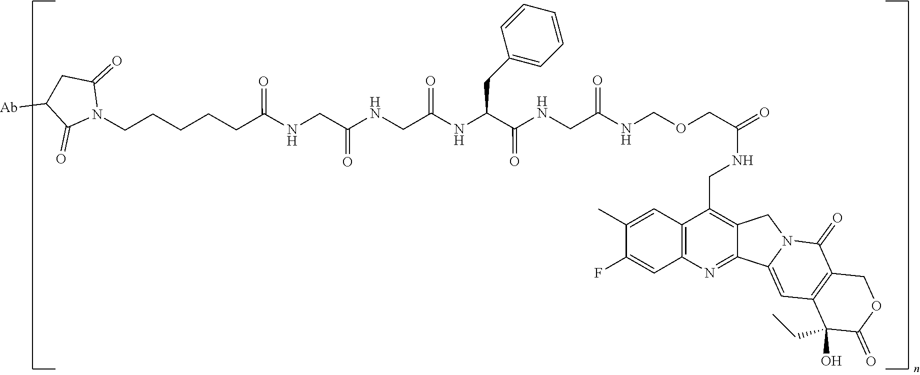

wherein: Ab is a nectin-4 antibody disclosed herein, and n is from about 1 to about 16.

In another aspect, provided herein is an ADC, wherein the ADC is of Formula X:

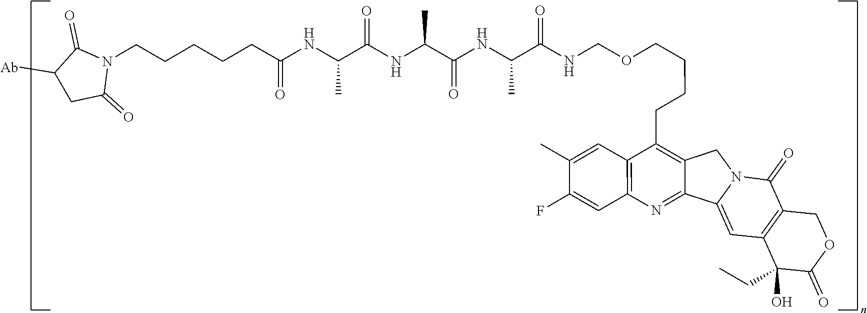

wherein: Ab is a nectin-4 antibody disclosed herein, and n is from about 1 to about 16.

In another aspect, provided herein is an ADC, wherein the ADC is of Formula XI:

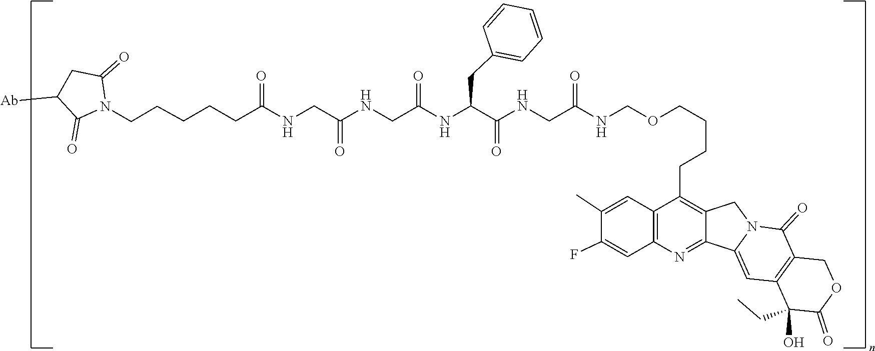

wherein: Abis a nectin-4 antibody disclosed herein, and n is from about 1 to about 16.

In another aspect, provided herein is an ADC, wherein the ADC is of Formula XII:

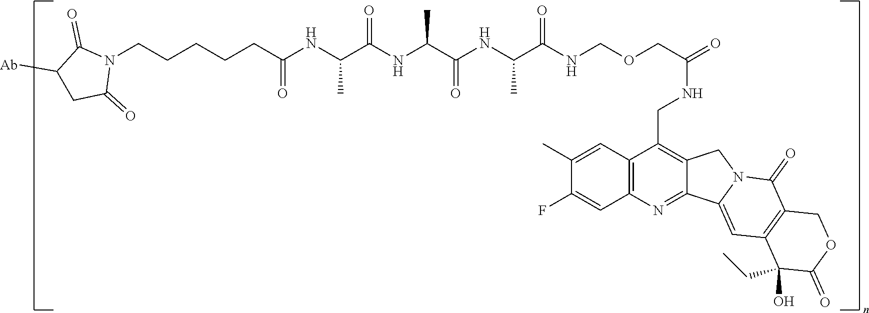

wherein: Ab is a nectin-4 antibody disclosed herein, and n is from about 1 to about 16.

In another aspect, provided herein is an ADC, wherein the ADC is of Formula XIII:

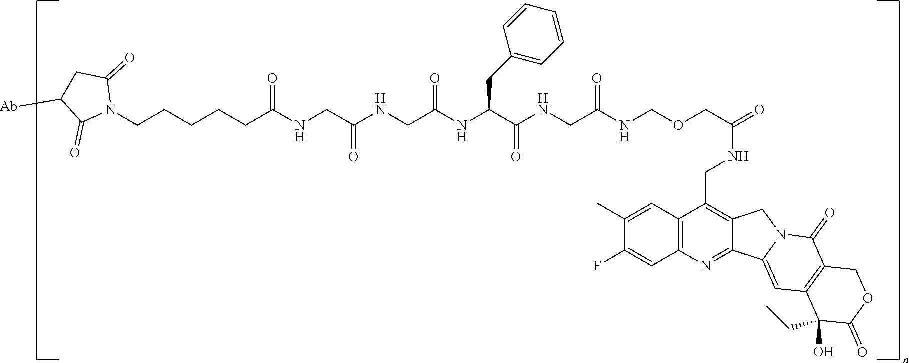

wherein: Ab is a nectin-4 antibody disclosed herein, and n is from about 1 to about 16.

In a further aspect, n is from about 2 to about 12. In another aspect, n is from about 2 to about 8. In another aspect, n is from about 4 to about 8. In another aspect, n is from about 8 to about 12. In another aspect, n is about 2. In another aspect, n is about 4. In another aspect, n is about 6. In another aspect, n is about 8. In another aspect, n is about 10. In another aspect, n is about 12.

In an aspect, disclosed herein, wherein the Ab comprises a HC comprising amino acids 2-444 of SEQ ID NO: 2 and a LC comprising amino acids 2-215 of SEQ ID NO: 3, and wherein n is about 8. In an aspect, disclosed herein, wherein the Ab comprises a HC comprising amino acids 2-444 of SEQ ID NO: 2 and a LC comprising amino acids 2-215 of SEQ ID NO: 3, and wherein n is about 4. In a further aspect, disclosed herein, wherein the Ab comprises a HC consisting of SEQ ID NO: 2 and a LC consisting of SEQ ID NO: 3.

In an aspect, disclosed herein, wherein the Ab comprises a HC comprising amino acids 2-444 of SEQ ID NO: 12 and a LC comprising amino acids 2-215 of SEQ ID NO: 13, and wherein n is about 8. In an aspect, disclosed herein, wherein the Ab comprises a HC comprising amino acids 2-444 of SEQ ID NO: 12 and a LC comprising amino acids 2-215 of SEQ ID NO: 13, and wherein n is about 4. In a further aspect, disclosed herein, wherein the Ab comprises a HC consisting of SEQ ID NO: 12 and a LC consisting of SEQ ID NO: 13.

In an aspect, disclosed herein, wherein the Ab comprises a HC comprising amino acids 2-443 of SEQ ID NO: 16 and a LC consisting of SEQ ID NO: 17, and wherein n is about 8. In an aspect, disclosed herein, wherein the Ab comprises a HC comprising amino acids 2-443 of SEQ ID NO: 16 and a LC consisting of SEQ ID NO: 17, and wherein n is about 4. In a further aspect, disclosed herein, wherein the Ab comprises a HC consisting of SEQ ID NO: 16 and a LC consisting of SEQ ID NO: 17.

In an aspect, disclosed herein, wherein the Ab comprising amino acids 2-447 of SEQ ID NO: 26 and a LC consisting of SEQ ID NO: 27, and wherein n is about 8. In an aspect, disclosed herein, wherein the Ab comprising amino acids 2-447 of SEQ ID NO: 26 and a LC consisting of SEQ ID NO: 27, and wherein n is about 4. In a further aspect, disclosed herein, wherein the Ab comprises a HC consisting of SEQ ID NO: 26 and a LC consisting of SEQ ID NO: 27.

In an aspect, disclosed herein, wherein the Ab comprising amino acids 2-446 of SEQ ID NO: 36 and a LC consisting of SEQ ID NO: 37, and wherein n is about 8. In an aspect, disclosed herein, wherein the Ab comprising amino acids 2-446 of SEQ ID NO: 36 and a LC consisting of SEQ ID NO: 37, and wherein n is about 4. In a further aspect, disclosed herein, wherein the Ab comprises a HC consisting of SEQ ID NO: 36 and a LC consisting of SEQ ID NO: 37.

In an aspect, disclosed herein, wherein the Ab comprising amino acids 2-446 of SEQ ID NO: 46 and a LC consisting of SEQ ID NO: 47, and wherein n is about 8. In an aspect, disclosed herein, wherein the Ab comprising amino acids 2-446 of SEQ ID NO: 46 and a LC consisting of SEQ ID NO: 47, and wherein n is about 4. In a further aspect, disclosed herein, wherein the Ab comprises a HC consisting of SEQ ID NO: 46 and a LC consisting of SEQ ID NO: 47.

In an aspect, disclosed herein, wherein the Ab comprising amino acids 2-446 of SEQ ID NO: 54 and a LC consisting of SEQ ID NO: 55, and wherein n is about 8. In an aspect, disclosed herein, wherein the Ab comprising amino acids 2-446 of SEQ ID NO: 54 and a LC consisting of SEQ ID NO: 55, and wherein n is about 4. In a further aspect, disclosed herein, wherein the Ab comprises a HC consisting of SEQ ID NO: 54 and a LC consisting of SEQ ID NO: 55.

In an aspect, disclosed herein, wherein the Ab comprising amino acids 2-450 of SEQ ID NO: 64 and a LC comprising amino acids 2-216 of SEQ ID NO: 65, and wherein n is about 8. In an aspect, disclosed herein, wherein the Ab comprising amino acids 2-450 of SEQ ID NO: 64 and a LC comprising amino acids 2-216 of SEQ ID NO: 65, and wherein n is about 4. In a further aspect, disclosed herein, wherein the Ab comprises a HC consisting of SEQ ID NO: 64 and a LC consisting of SEQ ID NO: 65.

In an aspect, disclosed herein, wherein the Ab comprising amino acids 2-450 of SEQ ID NO: 74 and a LC comprising amino acids 2-216 of SEQ ID NO: 75, and wherein n is about 8. In an aspect, disclosed herein, wherein the Ab comprising amino acids 2-450 of SEQ ID NO: 74 and a LC comprising amino acids 2-216 of SEQ ID NO: 75, and wherein n is about 4. In a further aspect, disclosed herein, wherein the Ab comprises a HC consisting of SEQ ID NO: 74 and a LC consisting of SEQ ID NO: 75.

### Conjugation to Anti-Nectin-4 Antibody

Methods to conjugate antibodies disclosed herein to the payloads disclosed herein are known in the art. In some methods, the antibody is conjugated to a linker in a first reaction, and then the antibody and linker are conjugated to the payload in a second reaction. In some methods, the antibody is conjugated to the payload or payload/linker in one reaction.

In some aspects of the present disclosure, a nectin-4 antibody described herein is covalently linked to a camptothecin analog described herein through the thiol group of one or more cysteine residues located on the nectin-4 antibody. In further aspects, the cysteine residue(s) used for conjugation are each an interchain disulfide cysteine residue. Known in the art are methods to control reduction of the interchain disulfides to allow for conjugation to those involved cysteines. In other aspects, the cysteine residue or residues used for conjugation are engineered into the antibody, separate from the ones used for interchain disulfides.

In other aspects of the present disclosure, a nectin-4 antibody described herein is covalently linked to a camptothecin analog described herein through the amino group of one or more lysine residues located on the nectin-4 antibody.

In an aspect, provided herein is an ADC, wherein connection of the cytotoxic agent, cytotoxic agent-self-immolative spacer, or cytotoxic agent-self-immolative spacer-linker to the antibody occurs through a thiol group on one or more cysteines of the antibody. In a further aspect, wherein the one or more cysteines are each a natural cysteine in the hinge region of the antibody.

### Drug-to-Antibody Ratio (DAR)

In the present disclosure, the drug loading in formulas is represented by n, which is the number of drug molecules per antibody (also known as drug-to-antibody ratio or DAR). Depending on the context, the subscript n represents the number of drug molecules attached to an individual antibody molecule and as such, is an integer value, or can represent an average drug load and, as such, can be an integer or non-integer value. An average drug load represents the average number of drug-linker molecules per antibody in a composition.

Higher DAR can produce more potent ADCs, but also, higher DAR can also result in destabilization, aggregation, increased off-target toxicity, and enhanced drug clearance from systemic circulation.

In aspects of the present disclosure, the average drug load when referring to a composition comprising a population of ADCs is from about 1 to about 16, about 2 to about 12, about 2 to about 10. In further aspects, the DAR is from about 2 to about 8. In a further aspect, the DAR is 4. In another aspect, the DAR is from about 6 to about 10. In a further aspect, the DAR is 8. The DAR in a preparation may be characterized by conventional means known in the art using technology such as mass spectroscopy, HIC, ELISA assay, or HPLC.

The present disclosure provides a method of producing an ADC, the method comprising the steps of:
(a) reducing a nectin-4 antibody disclosed herein with a reducing agent to produce a reduced nectin-4 antibody; and
(b) contacting the reduced nectin-4 antibody with a compound of the present disclosure to produce the conjugate, wherein the compound comprises one or more of Formulas I-IX. A further aspect, wherein the reducing agent is DTT or TCEP

The conjugates of the present disclosure, or salts thereof, may be readily prepared by a variety of procedures known to one of ordinary skill in the art, some of which are illustrated in the preparations and examples below. One of ordinary skill in the art recognizes that the specific synthetic steps for each of the routes described may be combined in different ways, or in conjunction with steps from different schemes, to prepare conjugates of the disclosure, or salts thereof. The product of each step can be recovered by conventional methods well known in the art, including extraction, evaporation, precipitation, chromatography, filtration, trituration, and crystallization. All substituents unless otherwise indicated, are as previously defined. The reagents and starting materials are readily available to one of ordinary skill in the art. The following preparations, examples, and assays further illustrate the disclosure, but should not be construed to limit the scope of the disclosure in any way.

### Therapeutic Applications

In another aspect, provided herein are methods of treating cancer, comprising administering to a patient in need thereof, an effective amount of a nectin-4 ADC or pharmaceutical composition described herein. In a further aspect, provided herein is a method of treating cancer, comprising administering to a patient in need thereof, an effective amount of an ADC or pharmaceutical composition described herein, wherein the cancer is bladder cancer, breast cancer, lung cancer, gastric cancer, colorectal cancer, pancreatic cancer, head and neck cancer, ovarian cancer, cervical cancer, or prostate cancer. In a further aspect, wherein the ADC or pharmaceutical composition described herein is administered in peri-operative, adjuvant, or neoadjuvant setting.

In a further aspect, provided is a method of treating cancer, wherein the cancer is bladder cancer. In a further aspect, provided is a method of treating cancer, wherein the cancer is urothelial carcinoma. In a further aspect, wherein the urothelial carcinoma is methastatic urothelial carcinoma (mUC). In a further aspect, provided is a method of treating cancer, wherein the cancer is breast cancer. In a further aspect, provided is a method of treating cancer, wherein the cancer is lung cancer. In a further aspect, provided is a method of treating cancer, wherein the cancer is gastric cancer. In a further aspect, provided is a method of treating cancer, wherein the cancer is colorectal cancer. In a further aspect, provided is a method of treating cancer, wherein the cancer is pancreatic cancer. In a further aspect, provided is a method of treating cancer, wherein the cancer is head and neck cancer. In a further aspect, provided is a method of treating cancer, wherein the cancer is ovarian cancer. In a further aspect, provided is a method of treating cancer, wherein the cancer is cervical cancer. In a further aspect, provided is a method of treating cancer, wherein the cancer is prostate cancer.

In a further aspect, the patient has relapsed after being administered enfortumab vedotin, or the patient has become refractory to enfortumab vedotin or to standard of care treatments. In a further aspect, the patient being treated with an ADC or pharmaceutical composition described herein is ineligible for treatment with enfortumab vedotin. In a further aspect, the patient being treated with an ADC or pharmaceutical composition described herein is naïve to treatment with enfortumab vedotin. In a further aspect, the patient being treated with an ADC or pharmaceutical composition described herein previously received a programmed death receptor-1 (PD-1) or programmed death-ligand 1 (PD-L1) inhibitor, and a platinum-containing chemotherapy in the neoadjuvant/adjuvant, locally advanced or metastatic setting. In a further aspect, the patient being treated with an ADC or pharmaceutical composition described herein has become refractory or has relapsed to enfortumab in combination with pembrolizumab, or nivolumab in combination with ipilimumab, or atezolizumab in combination with cisplatin/gemcitabine. In a further aspect, the patient being treated with an ADC or pharmaceutical composition described herein has become refractory or has relapsed on cisplatin or carboplatin in combination with gemcitabine. In a further aspect, the patient being treated with an ADC or pharmaceutical composition described herein in combination with a PD-1 inhibitor or PD-L1 inhibitor is ineligible for treatment with cisplatin-containing chemotherapy.

In a further aspect, provided are methods comprising the administration of an effective amount of an ADC or pharmaceutical composition described herein in simultaneous, separate, or sequential combination with one or more antitumor agents. In a further aspect, provided are methods comprising the administration of an effective amount of an ADC or pharmaceutical composition described herein in simultaneous, separate, or sequential combination with a PD-1 inhibitor or PD-L1 inhibitor.

In another aspect, provided herein are methods comprising administration of an effective amount of a nectin-4 ADC or pharmaceutical composition described herein in simultaneous, separate, or sequential combination with a FGFR compound. In a further aspect, wherein the cancer is urothelial carcinoma. In a further aspect, wherein the FGFR compound is erdafitinib, LOXO-435, futibatinib, vofatamab, bemarituzumab, derazantinib, infigratinib, pemigatinib, rogaratinib, FGF401, or pemigatinib.

In another aspect, provided herein is a nectin-4 ADC or pharmaceutical composition described herein, for use in therapy. In a further aspect, provided is an ADC or pharmaceutical composition described herein, for use in the treatment of cancer. In a further aspect, the cancer is urothelial carcinoma, breast cancer, lung cancer, gastric cancer, colorectal cancer, pancreatic cancer, head and neck cancer, ovarian cancer, cervical cancer, or prostate cancer. In a further aspect, the ADC or pharmaceutical composition described herein is for use in the peri-operative, adjuvant, or neoadjuvant setting.

In a further aspect, provided herein is a nectin-4 ADC or pharmaceutical composition described herein, for use in the treatment of bladder cancer. In a further aspect, provided herein is a nectin-4 ADC or pharmaceutical composition described herein, for use in the treatment of urothelial carcinoma. In a further aspect, wherein the urothelial carcinoma is methastatic urothelial carcinoma (mUC). In a further aspect, provided herein is an ADC or pharmaceutical composition described herein, for use in the treatment of breast cancer. In a further aspect, provided herein is an ADC or pharmaceutical composition described herein, for use in the treatment of lung cancer. In a further aspect, provided herein is an ADC or pharmaceutical composition described herein, for use in the treatment of gastric cancer. In a further aspect, provided herein is an ADC or pharmaceutical composition described herein, for use in the treatment of colorectal cancer. In a further aspect, provided herein is an ADC or pharmaceutical composition described herein, for use in the treatment of pancreatic cancer. In a further aspect, provided herein is an ADC or pharmaceutical composition described herein, for use in the treatment of head and neck cancer. In a further aspect, provided herein is an ADC or pharmaceutical composition described herein, for use in the treatment of ovarian cancer. In a further aspect, provided herein is an ADC or pharmaceutical composition described herein, for use in the treatment of cervical cancer. In a further aspect, provided herein is an ADC or pharmaceutical composition described herein, for use in the treatment of prostate cancer.

In a further aspect, provided herein is a nectin-4 ADC or pharmaceutical composition described herein, for use in the treatment of cancer, wherein the cancer has relapsed after treatment with enfortumab vedotin, or the cancer has become refractory to enfortumab vedotin. In a further aspect, provided herein is a nectin-4 ADC or pharmaceutical composition described herein, for use in the treatment of cancer, wherein the cancer has relapsed after treatment with enfortumab vedotin, or the cancer has become refractory to standard of care treatment. In a further aspect, provided herein is an ADC or pharmaceutical composition described herein, for use in the treatment of cancer, wherein prior use of enfortumab vedotin was contraindicated. In a further aspect, provided herein is an ADC or pharmaceutical composition described herein, for use in the treatment of cancer, wherein no prior use of enfortumab vedotin occurred. In a further aspect, provided herein is an ADC or pharmaceutical composition described herein, for use in the treatment of cancer, wherein prior use occurred for a PD-1 or PD-L1 inhibitor, and a platinum-containing chemotherapy in the neoadjuvant/adjuvant, locally advanced or metastatic setting, or wherein prior use occurred for enfortumab in combination with pembrolizumab, or nivolumab in combination with ipilimumab, or atezolizumab in combination with cisplatin/gemcitabine. In a further aspect, provided herein is an ADC or pharmaceutical composition described herein in simultaneous, separate, or sequential combination with a PD-1 inhibitor or PD-L1 inhibitor, for use in the treatment of cancer, wherein the cancer cannot be treated with cisplatin-containing chemotherapy.

In a further aspect, provided herein is an ADC or pharmaceutical composition described herein in simultaneous, separate, or sequential combination with one or more antitumor agents for use in the treatment of cancer. In a further aspect, with the antitumor agent is a PD-1 inhibitor or PD-L1 inhibitor.

In another aspect, provided herein is a nectin-4 ADC or pharmaceutical composition described herein in simultaneous, separate, or sequential combination with a FGFR compound for use in the treatment of cancer. In a further aspect, wherein the cancer is urothelial carcinoma. In a further aspect, wherein the FGFR compound is erdafitinib, LOXO-435, futibatinib, vofatamab, bemarituzumab, derazantinib, infigratinib, pemigatinib, rogaratinib, FGF401, or pemigatinib.

In another aspect, provided herein is the use of a nectin-4 ADC or pharmaceutical composition described herein for the manufacture of a medicament for the treatment of cancer. In a further aspect, provided herein is the use of an ADC or pharmaceutical composition described herein for the manufacture of a medicament for the treatment of cancer, wherein the cancer is bladder cancer, breast cancer, lung cancer, gastric cancer, colorectal cancer, pancreatic cancer, head and neck cancer, ovarian cancer, cervical cancer, or prostate cancer.

In a further aspect, provided herein is the use of a nectin-4 ADC or pharmaceutical composition described herein for the manufacture of a medicament for the treatment of cancer, wherein the cancer has relapsed after treatment with enfortumab vedotin, or the cancer has become refractory to enfortumab vedotin or to standard of care treatment. In a further aspect, provided herein is the use of an ADC or pharmaceutical composition described herein for the manufacture of a medicament for the treatment of cancer, wherein prior use of enfortumab vedotin was contraindicated. In a further aspect, provided herein is the use of an ADC or pharmaceutical composition described herein for the manufacture of a medicament for the treatment of cancer, wherein there was no prior use of enfortumab vedotin. In a further aspect, provided herein is the use of an ADC or pharmaceutical composition described herein for the manufacture of a medicament for the treatment of cancer, wherein prior use of a PD-1 or PD-L1 inhibitor, and a platinum-containing chemotherapy in the neoadjuvant/adjuvant, locally advanced or metastatic setting. In a further aspect, provided herein is the use of an ADC or pharmaceutical composition described herein for the manufacture of a medicament for the treatment of cancer, wherein the cancer cannot be treated with cisplatin-containing chemotherapy and wherein said medicament is to be administered simultaneously, separately, or sequentially with a PD-1 inhibitor or PD-L1 inhibitor.

In a further aspect, provided herein is the use of an ADC or pharmaceutical composition described herein in the manufacture of a medicament for the treatment of cancer wherein said medicament is to be administered simultaneously, separately, or sequentially with one or more antitumor agents. In a further aspect, provided herein is the use of an ADC or pharmaceutical composition described herein in the manufacture of a medicament for the treatment of cancer wherein said medicament is to be administered simultaneously, separately, or sequentially with a PD-1 inhibitor or PD-L1 inhibitor.

In another aspect, provided herein is the use of a nectin-4 ADC or pharmaceutical composition described herein in the manufacture of a medicament for the treatment of cancer wherein said medicament is to be administered simultaneously, separately, or sequentially with a FGFR compound. In a further aspect, wherein the cancer is urothelial carcinoma. In a further aspect, wherein the FGFR compound is erdafitinib, LOXO-435, futibatinib, vofatamab, bemarituzumab, derazantinib, infigratinib, pemigatinib, rogaratinib, FGF401, or pemigatinib.

In a further aspect, the bladder cancer is urothelial carcinoma, squamous cell carcinoma, or adenocarcinoma. In a further aspect, the bladder cancer is noninvasive, non-muscle-invasive, or muscle invasive. In a further aspect, the bladder cancer is muscle-invasive bladder cancer and after a cystectomy. In a further aspect, the bladder cancer is of the bladder, renal pelvis, ureter, or urethra. In a further aspect, the breast cancer is HR-positive, HER2-negative breast cancer, or triple negative breast cancer (TNBC). In a further aspect, the breast cancer is ductal or lobular. In a further aspect, the lung cancer is squamous non-small cell lung cancer (NSCLC) or non-squamous NSCLC. In a further aspect, the lung cancer is squamous, adenocarcinoma, or small cell carcinoma. In a further aspect, the prostate cancer is metastatic castration-resistant prostate cancer. In a further aspect, the gastric cancer is esophageal cancer or gastroesophageal junction cancer. In a further aspect, the ovarian cancer is serous or mucinous. In a further aspect, the ovarian cancer is fallopian or peritoneal.

In a further aspect, the antitumor agents may be chemotherapeutic therapeutic agents, including platinum-containing chemotherapy, and/or may include cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), gemcitabine, topotecan, liposomal irinotecan, pemetrexed, methotrexate, vinblastine, and cetuximab. In a further aspect, the chemotherapeutic agents is a combination of cisplatin (or carboplatin) and gemcitabine, including for bladder cancer. In a further aspect, the antitumor agents may be immuno-oncology agents, including those selected from the group consisting of nivolumab, ipilimumab, pidilizumab, pembrolizumab, tremelimumab, urelumab, lirilumab, atezolizumab, epacadostat, and durvalumab.

### Pharmaceutical Compositions and Methods of Administration

The antibodies or ADCs described herein can be formulated as pharmaceutical compositions administered by any route which makes the antibody or ADC bioavailable including, for example, oral, topical, or subcutaneous administration.

Also provided herein is a pharmaceutical composition comprising an antibody or ADC provided herein and one or more agents selected from the group consisting of a physiologically acceptable carrier, a diluent, an excipient, and an auxiliary.

The antibodies or ADCs of the present disclosure, or pharmaceutical compositions comprising the same, may be administered by parenteral routes (e.g., subcutaneous and intravenous). An antibody or ADC of the present disclosure may be administered to a patient alone with pharmaceutically acceptable carriers, diluents, or excipients in single or multiple doses. Pharmaceutical compositions described herein can be prepared by methods well known in the art (e.g., Remington: The Science and Practice of Pharmacy, 22nd ed. (2012), A. Loyd et al., Pharmaceutical Press) and comprise an antibody or ADC, as disclosed herein, and one or more pharmaceutically acceptable carriers, diluents, or excipients.

In an aspect, disclosed herein is a pharmaceutical composition comprising an antibody disclosed herein and one or more pharmaceutically acceptable carriers, diluents, or excipients. In an aspect, disclosed herein is a pharmaceutical composition comprising an ADC disclosed herein and one or more pharmaceutically acceptable carriers, diluents, or excipients.

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

The terms “bind” and “binds” as used herein, are intended to mean, unless indicated otherwise, the ability of a protein or molecule to form a chemical bond or attractive interaction with another protein or molecule, which results in proximity of the two proteins or molecules as determined by common methods known in the art.

As used herein, the term “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

As used herein, the terms “treating”, “treatment”, or “to treat” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms.

The term “patient”, as used herein, refers to a human patient.

Certain abbreviations are defined as follows: “ACN” refers to acetonitrile; “DCM” refers to dichloromethane; “DIPEA” refers to N,N-diisopropylethylamine; “DBU” refers to 1,8-diazabicyclo[5.4.0]undec-7-ene; “DMTMM” refers to (4-(4,6-dimethoxy-1,3,5-triazin-2-yl)-4-methyl-morpholinium chloride; “DMAC” refers to dimethylacetamide; “DMF” refers to N, N-dimethylformamide; “DTT” refers to dithiothreitol; “EtOAc” refers to ethyl acetate; “EDTA” refers to ethylenediaminetetraacetic acid, “FA” refers to formic acid; “HMPA” refers to hexamethylphosphoramide; “h” refers to hour; “HEPES” refers to (N-2-hydroxyethylpiperazine-N′-2-ethanesulfonic acid); “NMM” refers to N-methylmorpholine; “NMP” refers to (N-methyl-2-pyrrolidone); “Su” refers to succinimide; “PPTS” refers to pyridinium p-toluenesulfonate; “THF” refers to tetrahydrofuran; “TsOH” refers to p-toluenesulfonic acid; and “TCEP” refers to (tris(2-carboxyethyl)phosphine).

### EXAMPLES

### Example 1: Generation of Nectin-4 Antibodies

The amino acid sequences of the CDRs, the variable regions, the complete heavy chain and light chain of Antibodies 1-8, and the nucleotide sequences encoding the same, are listed below in the section entitled “Amino Acid and Nucleotide Sequences.” In addition, the SEQ ID NOs for the CDRs, light chain, heavy chain, light chain variable region, and heavy chain variable region of Antibodies 1-8 are shown in Tables 1 and 2.

The anti-nectin-4 antibodies of the present disclosure, including, but not limited to Abs 1-8, can be expressed and purified essentially as follows. Antibodies are expressed in an appropriate host cell, such as HEK293 or CHO, either transiently or stably transfected with an expression system for secreting antibodies using an optimal predetermined HC:LC vector ratio or a single vector system encoding both HC and LC. The expression plasmid contains cDNA versions of the LC and HC genes for an antibody (for example, as presented in Table 3); and are expressed from a commonly-used and suitable construct for this purpose, such as one based on human cytomegalovirus major immediate early promoters.

Medium, into which an antibody of the present disclosure has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized. Various methods of protein purification may be employed, and such methods are known in the art and described, for example, in 30 Deutscher, Methods in Enzymology 182: 83-89 (1990) and Scopes, Protein Purification: Principles and Practice, 3rd Edition, Springer, NY (1994). Antibodies may be immediately frozen at −70° C. or stored at 2-8° C. for several months, or may be lyophilized, or preserved in 4° C. for immediate use.

TABLE 1
SEQ ID Nos for CDR amino acid sequences of exemplified human
nectin-4 antibodies

| Nectin-4 | SEQ ID No. for CDR Sequence | | | | | |
|---|---|---|---|---|---|---|
| Antibody | HCDR1 | HCDR2 | HCDR3 | LCDR1 | LCDR2 | LCDR3 |
| Ab1 | 4 | 5 | 6 | 7 | 8 | 9 |
| Ab1a | 4 | 5 | 6 | 7 | 8 | 9 |
| Ab2 | 18 | 19 | 20 | 21 | 22 | 23 |
| Ab3 | 28 | 29 | 30 | 31 | 32 | 33 |
| Ab4 | 38 | 39 | 40 | 41 | 42 | 43 |
| Ab5 | 48 | 49 | 50 | 41 | 42 | 51 |
| Ab6 | 56 | 57 | 58 | 59 | 60 | 61 |
| Ab7 | 66 | 67 | 68 | 69 | 70 | 71 |
| Ab8 | 76 | 77 | 78 | 69 | 70 | 79 |

TABLE 2
SEQ ID Nos for exemplified human nectin-4 antibodies

| Nectin-4 | | | | |
|---|---|---|---|---|
| Antibody | HC | LC | HCVR | LCVR |
| Ab1 | 2 | 3 | 10 | 11 |
| Ab1a | 12 | 13 | 14 | 15 |
| Ab2 | 16 | 17 | 24 | 25 |
| Ab3 | 26 | 27 | 34 | 35 |
| Ab4 | 36 | 37 | 44 | 45 |
| Ab5 | 46 | 47 | 52 | 53 |
| Ab6 | 54 | 55 | 62 | 63 |
| Ab7 | 64 | 65 | 72 | 73 |
| Ab8 | 74 | 75 | 80 | 81 |

TABLE 3
SEQ ID Nos for DNA sequences for heavy and light
chains of exemplified human nectin-4 antibodies

| Nectin-4 | | |
|---|---|---|
| Antibody | HC | LC |
| Ab1 | 82 | 83 |
| Ab1a | 84 | 85 |
| Ab2 | 86 | 87 |
| Ab3 | 88 | 89 |
| Ab4 | 90 | 91 |
| Ab5 | 92 | 93 |
| Ab6 | 94 | 95 |
| Ab7 | 96 | 97 |
| Ab8 | 98 | 99 |

### Example 2: Generation of Nectin-4 ADCs

### Synthesis of Camptothecin Analog

Essentially as prepared in ACS Med. Chem. Lett. 2019, 10, 1386-1392 and WO2020219287, camptothecin analogs of the present disclosure, such as A1, can be synthesized as follows:

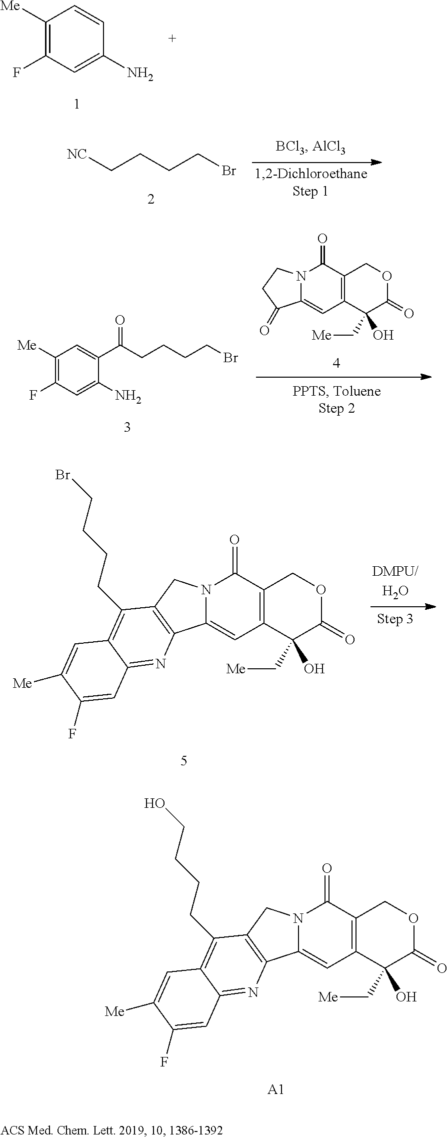

Step 1: To a flask containing anhydrous 1,2-dichloroethane (50 mL) was added 1 M BCl₃ in DCM (9.95 mL, 9.95 mmol) then cooled to 0° C. with an ice H₂O bath. 3-Fluoro-4-methylaniline 1 (1.56 g, 12.4 mmol) was added in portions then stirred at 0° C. for 10 min, then 5-bromovaleronitrile 2 (1.72 mL, 14.9 mmol) was added followed by the addition of AlCl₃ (2.16 g, 16.2 mmol). The ice bath was removed, and the reaction solution was gradually warmed to room temperature. After stirring at room temperature for 10 min, the mixture was heated at reflux for 39 h. The solution was cooled to room temperature and cold H₂O (25 mL) was added slowly followed by the addition of 5% HCl aqueous solution. After 30 min, the solution was diluted with DCM (50 mL). The organic layer was washed with H₂O and brine, dried over anhydrous Na₂SO₄ then filtered. The filtrate was concentrated under reduced pressure. The residue was purified by reverse phase chromatography using the following conditions: Column: C18 (100 g); eluted with 25% ACN in H₂O for 5 min then switched to a gradient of 25% to 95% ACN in H₂O for 15 min then 95% ACN in H₂O for 5 min to give compound 3 as an off-white solid (1.42 g, 40%).

Step 2: Compound 3 (3.15 g, 15.64 mmol), Compound 4 (3.92 g, 14.89 mmol) in toluene (200 mL) and PPTS (0.037 g, 0.15 mmol) were suspended in a 50 mL flask equipped with a reflux condenser containing anhydrous toluene (10 mL). The reaction was heated at reflux for 40 h with magnetic stirring under an argon atmosphere overnight then allowed to cool to room temperature. The mixture was filtered, and the solids were washed with toluene (5 mL) to give 5 (4.74 g, 74%).

Step 3: A solution of compound 5 (0.860 g, 1.67 mmol) in HMPA (5 mL) and deionized water (0.9 mL) was heated at 101° C. for 18 h. Upon cooling to room temperature, the solution was loaded on a C18 cartridge and purified by reverse phase chromatography using the following conditions: Column: C18 (30 g); eluted with 25% ACN in H₂O, then switched to a gradient of 25% to 95% ACN in H₂O for 15 min then 95% ACN in H₂O for 5 min to give a mixture. The mixture was further purified by silica gel chromatography, eluted with a linear gradient of 0 to 20% MeOH in CH₂Cl₂ over 15 min to give compound A1 as an off-white solid (0.392 g, 51% yield).

### Synthetic Scheme of Linker+Self-Immolative Unit+Camptothecin Analog

Essentially as prepared in ACS Med. Chem. Lett. 2019, 10, 1386-1392 and WO2020219287, camptothecin analogs of the present disclosure can be conjugated to the linker (with maleimide chemistry) and self-immolative unit as follows:

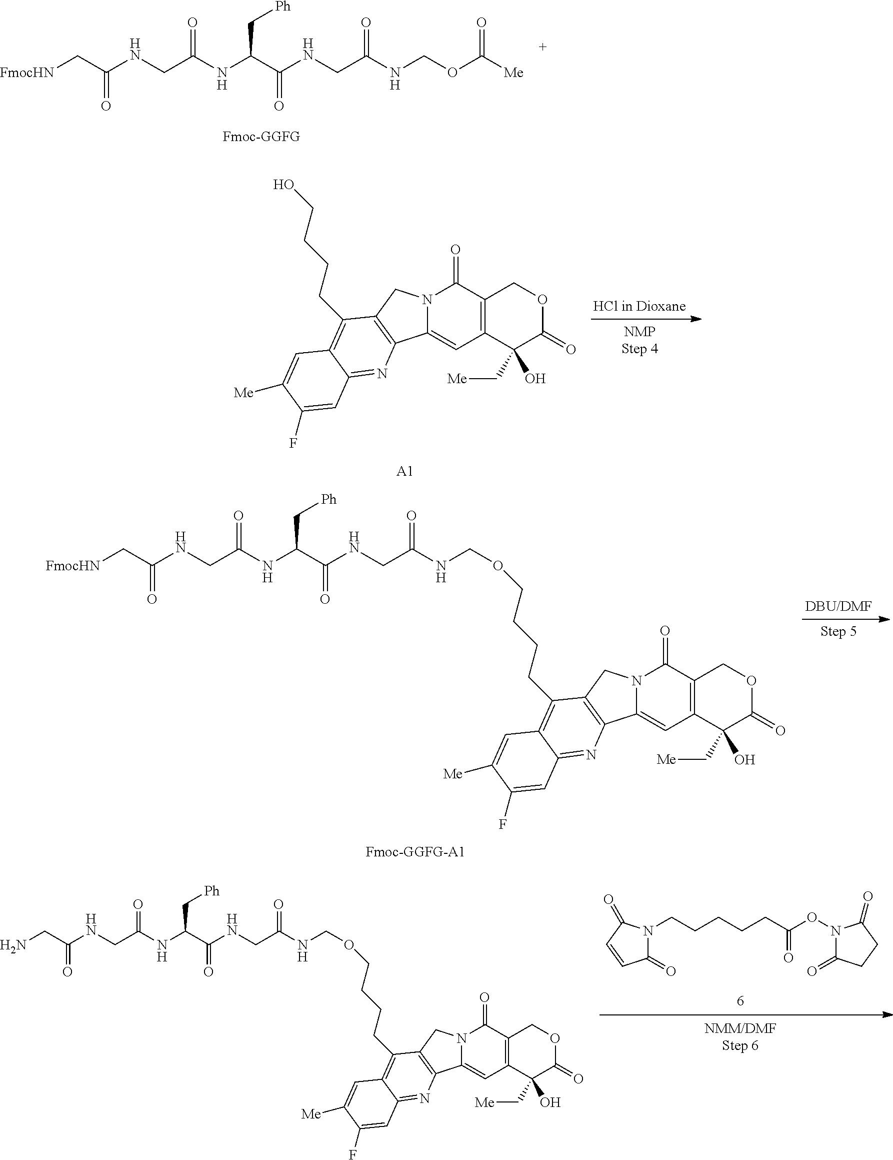

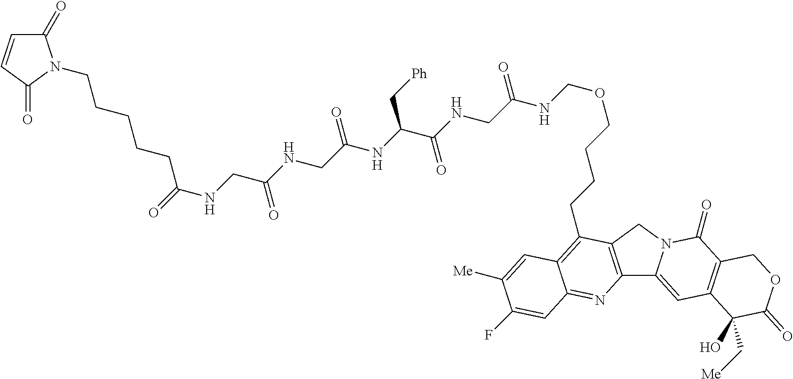 [SMILES: CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)CCCCCN1C(=O)C=CC1=O]

Step 4: A mixture of Fmoc-GGFG (1 g, 1.59 mmol, 1.5 equiv.), A1 (0.48 mg, 1.06 mmol, 1 equiv.) and 4 Å molecular sieve (3 g, 3× weight of A1) in NMP was treated with HCl (4M, 4.5 equiv.) in 1,4-dioxane at 20° C. The reaction was quenched by the addition of H2O. The solids were removed by filtration and the filtrate was extracted with EtOAc. The organic layer was dried over Na₂SO₄, filtered, and concentrated under reduced pressure. The residue was purified by normal phase chromatography (SiO₂, 0-10% MeOH in DCM) to give Fmoc-GGFG-A1 (0.75 g, 69% yield).

Step 5: Fmoc-GGFG-A1 (0.75 g, 0.73 mmol, 1 equiv.) in DMF was treated with DBU (0.11 mL, 0.77 mmol, 1.05 equiv.) at 0° C. The reaction was quenched by the addition of TsOH (0.25 g, 1.47 mmol, 2 equiv.). The resulting mixture was stirred at 0° C. for 4 h then used directly in the next step without further purification.

Step 6: The crude mixture of GGFG-A1 (0.73 mmol, 1 equiv.) and TsOH in DMF was basified to pH 7 with NMM at 0° C., followed by the addition of more NMM (0.16 mL, 1.47 mmol, 2 equiv.) and Compound 6 (0.283 g, 0.917 mmol, 1.25 equiv.). The resulting mixture was stirred at 0° C. for 4-6 hours, then quenched and purified by normal phase chromatography (SiO2, 0-10% MeOH in DCM) to give mc-GGFG-A1 (0.51 g, 70% yield).

Camptothecin analogs of the present disclosure can be conjugated to the linker (with bromoacetamide chemistry) and self-immolative unit as follows:

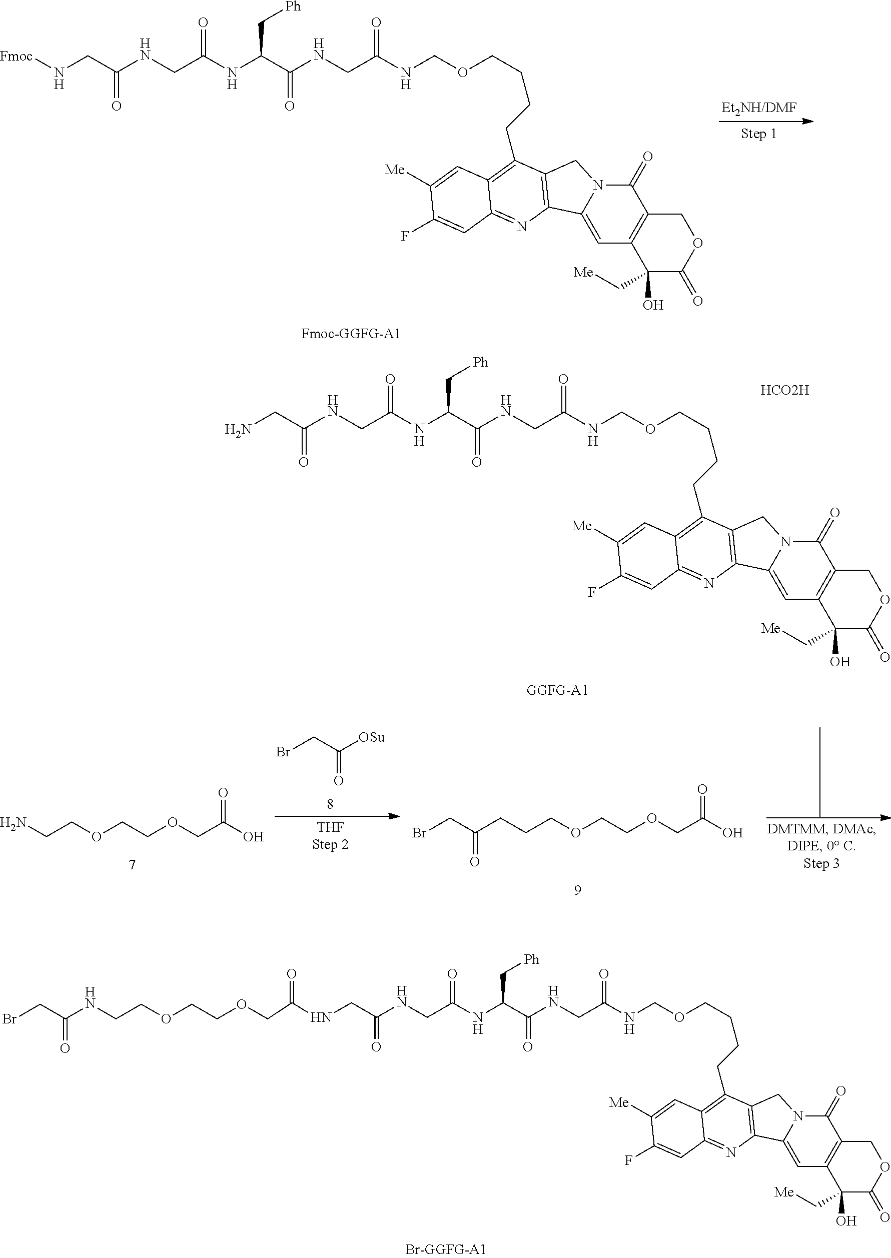

Step 1: To a solution of Fmoc-GGFG-A1 (0.5 g, 0.489 mmol) in DMF (5 ml) was added Et₂NH (0.025 g, 0.34 mmol). The mixture was stirred between 15-25° C. for 1 h. The reaction was then quenched by the addition of THF, followed by concentration under reduced pressure. The residue was purified by reverse phase reverse phase chromatography using the following conditions: Column: C18, 150*30 mm*5 μm; eluted with 22-45% ACN in H₂O (0.225% FA to give GGFG-A1 as the FA salt (0.196 g, 47% yield).

Step 2: To a solution of Compound 7 (2.0 g, 12.26 mmol) in 20 ml THF at 0° C. was added Compound 8 (2.9 g, 12.29 mmol). The reaction mixture was allowed to warm to 20° C. The stirring was continued at 20° C. for 18 h, followed by the addition of more Compound 8 (0.5 g, 3.06 mmol). After being stirred for additional 4 h at 20° C., the reaction was concentrated under reduced pressure. The residue was purified by reverse phase chromatography; Column: C18 column, 150*30 mm*5 μm, eluted with 22-45% ACN in H₂O (0.225% FA to give Compound 9 (1.15 g, 33% yield)

Step 3: A mixture of Compound 9 (430 mg, 1.51 mmol) and DMTMMT (340 mg, 1.04 mmol) in DMAC (11 ml) was cooled to 0° C. A mixture of GGFG-A1 (0.500 g, 0.63 mmol) and DIPEA (0.090 g, 0.70 mmol) in DMAC (4 ml) was added dropwise. The stirring was continued at 0° C. for 0.5 h, followed by addition of DCM (300 mL). The mixture was washed with aq 10% NaBr (3×50 ml). The organic layer was dried over Na₂SO₄, filtered, and concentrated between 0-10° C. under reduced pressure. The residue was purified by reverse phase chromatography; Column: C18 column, 150*30 mm*5 μm; eluted with 15-45% ACN in H₂O (0.225% FA) to give Br-GGFG-A1 (0.23 g, 34.7% yield).

Part I. Preparation of ADCs with Maleimide Linker-Payload

To prepare antibody-drug conjugates with eight drugs per antibody, the IgG1 antibody is fully reduced using a reducing reagent such as DTT or TCEP at 6 to 8 molar equivalents at 37° C. for 2 h. The reduced antibody is then buffer exchanged using a PD-10 desalting column with 50 mM HEPES with 2 mM EDTA at pH 7.0, and the eluent is adjusted with HEPES buffer to a protein concentration between 5-10 mg/ml. An excess of linker-payloads, such as 10 molar equivalents, is added for 1 hour, and the conjugation reaction may be stopped by adding a substantial excess of L-cysteine, such as 6 molar equivalents. The resulting mixture of ADCs may be purified on a PD-10 desalting column equilibrated in 25 mM histidine, 9% sucrose at pH 5.5, followed by 3 spin cycles with a 30 kDa MWCO centrifugal unit to remove any unreacted linker-payload related species. Finally, the resulting ADC may be sterile filtered through a 0.2 μM filter and stored at 4° C. or −80° C. for future use.

Part II. Preparation of ADCs with Bromoacetyl Linker-Payload

To prepare antibody-drug conjugates with eight drugs per antibody, the IgG1 antibody is fully reduced using a reducing reagent such as DTT or TCEP at 6 to 8 molar equivalents at 37° C. for 2 h. The reduced antibody is then buffer exchanged using a PD-10 desalting column with 50 mM HEPES with 2 mM EDTA at pH 7.4, and the eluent is adjusted with HEPES buffer to a protein concentration between 5-10 mg/ml. An excess of linker-payloads, such as 12 molar equivalents, is added for 2-3 hrs., and the conjugation reaction may be stopped by adding a substantial excess of L-cysteine, such as 10 molar equivalents. The resulting mixture of ADCs may be purified on a PD-10 desalting column equilibrated in 25 mM Histidine 9% Sucrose at pH 5.5, followed by 3 spin cycles with a 30 kDa MWCO centrifugal unit to remove any unreacted linker-payload related species. Finally, the resulting ADC may be sterile filtered through a 0.2 μM filter and stored at 4° C. or −80° C. for future use.

### Example 3: Antibody Binding Affinity, Cross-Reactivity, and Selectivity

### Characterizing the Human and Cross-Species Binding of Nectin-4 ADCs by Surface Plasmon Resonance

A Biacore 8K+ instrument (Cytiva, Marlborough, MA) was used to determine kinetics and affinity parameters for the binding interactions of the nectin-4 ADCs (exemplified herein with each of Abs1-8 conjugated as in Formula XI, and with a DAR of 8) with recombinant HIS-tagged human (Acro Biosystems Cat. No. NE4-H52H3, Newark, DE), cyno (Acro Biosystems, Cat. No. NE4-C52H4), and rat (R&D Systems, Minneapolis, MN, Cat. No. 9997-N4-050).

An anti-human Fc sensor surface was prepared by amine-coupling of goat anti-human IgG Fc (Southern Biotech Cat. No. 2014-01, Birmingham, AL) to a Biacore Series S CM4 (Cytiva Cat. No. BR-100534) sensor surface at 25° C. For immobilization, a running buffer of 10 mM HEPES, 150 mM NaCl, 0.05% Tween-20, pH 7.4 was used. Flow cells 1 & 2 of all 8 channels were activated with a 1:1 (v/v) mixture of 400 mM 1-Ethyl-3-(3-dimethylaminopropyl) carbodiimide hydrochloride (EDC) and 100 nM N-Hydroxysuccinimide (NHS) at a flow rate of 10 uL/min for 7 minutes. Then the anti-human IgG Fc capture reagent was coupled to the sensor surface (diluted to 50 μg/mL in 10 mM Acetate pH 4.5 buffer) by injecting it in all flow cells and channels at a flow rate of 10 uL/min for 7 minutes. Remaining active groups were blocked by injecting 100 mM ethylenediamine (in 200 mM Borate buffer, pH 8.5) in all flow cells and channels at a flow rate of 10 uL/min for 7 minutes. All channels and flow cells were then preconditioned using three consecutive 1-minute injections of 75 mM phosphoric acid at 10 uL/min.

For kinetics/affinity analysis, the running and sample dilution buffer was 10 mM sodium phosphate, 150 mM NaCl, 0.05% Tween-20, pH 7.4, 1 mg/mL bovine serum albumin (BSA) and the analysis temperature was 37° C.

In each analysis cycle, a different ADC was captured on flow cell 2 of each channel by injecting it at 5 μg/mL and 10 uL/min. After capture, the same analyte was injected on flow cells 1 and 2 in all 8 channels for 2 minutes at 30 uL/n and dissociation was monitored for 10 minutes. After dissociation, all surfaces were regenerated with three 1-minute injections of 5 mM phosphoric acid at 10 uL/min. ADC capture and analyte cycles were repeated to obtain analyte binding of each ADC at concentrations of 0, 2.5, 7.4, 22, 67, 200 and 600 nM nectin-4.

Sensorgram data were fit globally with the default 1:1 binding model in Biacore Insight Evaluation Software v3.0.12.15655. Kinetics and affinity parameters for the ADCs are shown in Table 4.

TABLE 4
Kinetics and affinity parameters for ADC/Nectin-4 interactions at 37° C.

| Ab in the | | | kₐ | k_{d} | K_{D} |
|---|---|---|---|---|---|
| ADC | Antigen | n | (1/Ms) | (1/s) | (nM) |
| Ab4 | human | 3 | (3.59 ± 0.80) × 10⁵ | (7.32 ± 1.19) × 10⁻² | 204 ± 56 |
| | Nectin-4 | | | | |
| Ab5 | human | 3 | (8.78 ± 1.07) × 10⁵ | (3.46 ± 0.48) × 10⁻³ | 3.94 ± 0.73 |
| | Nectin-4 | | | | |
| Ab1 | human | 3 | (2.27 ± 1.10) × 10⁵ | (3.78 ± 0.99) × 10⁻² | 166 ± 92 |
| | Nectin-4 | | | | |
| Ab3 | human | 3 | (4.24 ± 0.76) × 10⁵ | (4.37 ± 1.56) × 10⁻⁴ | 1.03 ± 0.41 |
| | Nectin-4 | | | | |
| Ab2 | human | 3 | (1.02 ± 0.34) × 10⁶ | (2.14 ± 0.66) × 10⁻² | 21.0 ± 9.6 |
| | Nectin-4 | | | | |
| Ab6 | human | 3 | (1.78 ± 0.41) × 10⁶ | (1.63 ± 0.25) × 10⁻¹ | 91.8 ± 25.5 |
| | Nectin-4 | | | | |
| Ab8 | human | 3 | (6.94 ± 1.14) × 10⁵ | (3.38 ± 0.75) × 10⁻² | 48.7 ± 13.5 |
| | Nectin-4 | | | | |
| Ab1a | human | 2 | 2.83 × 10⁵, | 4.02 × 10⁻², | 142, 72.5 |
| | Nectin-4 | | 4.24 × 10⁵ | 3.08 × 10⁻² | |
| Ab7 | human | 2 | 5.91 × 10⁵, | 2.43 × 10⁻², | 41.2, 37.6 |
| | Nectin-4 | | 5.64 × 10⁵ | 2.12 × 10⁻² | |
| Ab4 | cyno | 1 | 3.80 × 10⁵ | 7.24 × 10⁻² | 191 |
| | Nectin-4 | | | | |
| Ab5 | cyno | 1 | 1.06 × 10⁶ | 3.30 × 10⁻³ | 3.10 |
| | Nectin-4 | | | | |
| Ab1 | cyno | 1 | heterogeneous kinetics* | | |
| | Nectin-4 | | | | |
| Ab3 | cyno | 1 | 4.32 × 10⁵ | 4.26 × 10⁻⁴ | 0.986 |
| | Nectin-4 | | | | |
| Ab2 | cyno | 1 | 1.01 × 10⁶ | 2.00 × 10⁻² | 19.8 |
| | Nectin-4 | | | | |
| Ab6 | cyno | 1 | heterogeneous kinetics* | | |
| | Nectin-4 | | | | |
| Ab8 | cyno | 1 | 1.25 × 10⁶ | 5.24 × 10⁻² | 42.1 |
| | Nectin-4 | | | | |
| Ab1a | cyno | 1 | 3.10 × 10⁵ | 2.60 × 10⁻² | 83.9 |
| | Nectin-4 | | | | |
| Ab7 | cyno | 1 | 1.18 × 10⁶ | 4.64 × 10⁻² | 39.2 |
| | Nectin-4 | | | | |
| Ab4 | rat Nectin-4 | 1 | weak binding | | |
| Ab5 | rat Nectin-4 | 1 | 6.36 × 10⁵ | 1.99 × 10⁻² | 31.3 |
| Ab1 | rat Nectin-4 | 1 | 1.85 × 10⁵ | 6.12 × 10⁻² | 331 |
| Ab3 | rat Nectin-4 | 1 | 1.71 × 10⁵ | 9.03 × 10⁻⁴ | 5.28 |
| Ab2 | rat Nectin-4 | 1 | 6.95 × 10⁵ | 7.53 × 10⁻² | 108 |
| Ab6 | rat Nectin-4 | 1 | 9.25 × 10⁵ | 1.05 × 10⁻¹ | 114 |
| Ab8 | rat Nectin-4 | 1 | 4.82 × 10⁵ | 1.65 × 10⁻² | 34.4 |
| Ab1a | rat Nectin-4 | 1 | 2.52 × 10⁵ | 4.53 × 10⁻² | 180 |
| Ab7 | rat Nectin-4 | 1 | 4.04 × 10⁵ | 2.96 × 10⁻² | 73.1 |

*Binding is clearly observed; however, data exhibits kinetic heterogeneity resulting in a poor fit to the 1:1 binding model.
kₐ is the association rate constant, k_{d} is the dissociation rate constant, K_{D} is the equilibrium dissociation constant (calculated using K_{D} = k_{d}/kₐ), and n is the number of replicates. For n = 1 or 2, the replicate values are shown. For n = 3, mean ± standard deviations are shown.

### Cell Surface Binding of Nectin-4 Antibodies on Cell Lines Expressing the Nectin-4 Receptor

A panel of nine nectin-4 antibodies were tested for cell surface binding to two nectin-4 expressing tumor cell lines. Cell lines were selected to represent high and low receptor densities with SUM190PT tumor cells representing high endogenous expression, and NCI-H1781 tumor cells representing lower endogenous expression. SUM190PT and NCI H1781 cells were determined to have antibody binding capacity of 108,000 and 20,000 respectively (using MESF quantification kit, Bangs Laboratories). T24 parental cells were selected as the nectin-4 negative cell line. Antibody binding was quantified via flow cytometry and both the EC₅₀ of the binding curves and the maximum binding MFI for each antibody were recorded.

Cells were dissociated using non-enzymatic dissociation buffer for 5 min at 37° C. Cells were counted and aliquoted to a V-bottom polypropylene 96-well plate at 10⁵ cells/well. Cells were centrifuged at 1800 rpm×5 min and the supernatant discarded. An 11-point antibody dilution series was prepared in assay buffer (1×PBS containing 1% BSA and 0.09% Sodium Azide) starting at 300 nM and diluting 1:4 down. The dilution series was added to cells at 100 uL/well and mixed by pipetting. Several untreated control wells/cell line were prepared in assay buffer alone. Cells and antibodies were incubated at 4° C. on an orbital shaker for 1 hour. After incubation, the assay plates were centrifuged and washed twice with 300 uL/well of assay buffer. Cell pellets were then stained with a 1:500 dilution of Alexa647 conjugated mouse anti-human IgG secondary antibody in assay buffer, at 100 uL/well. Assay plates were incubated shaking in the dark at 4° C. for 1 hour. After incubation, plates were centrifuged, and the cells washed twice with 300 uL/well of assay buffer. A 1:5000 solution of Zombie Green viability marker (BioLegend) in 1×PBS was aliquoted to the cells at 100 uL/well and plates were incubated in the dark for 10 min shaking at 4° C. Cell pellets were then washed 1× with assay buffer and fixed with 200 uL/well of 4% paraformaldehyde in 1×PBS in the dark at room temperature for 15 min. Cells were centrifuged and washed 1× before resuspending in 65 μL of assay buffer for acquisition on a Sartorious iQue HTFC Cytometer.

Cells were acquired on a Sartorious iQue HTFC Cytometer and FCS files generated using ForeCyt standard edition (v. 6.2.6652). FCS files were then analyzed on FlowJo (v10.8.1). Debris was excluded from analysis by Forward scatter (FSC) vs side scatter (SSC) gating and single cells were selected via forward scatter area (FSC-A) vs height (FSC—H) gating. Finally, dead cells positive for staining with Zombie Green were excluded and the MFIs of live, Alexa647 positive cells were quantified. Average unstained cell autofluorescence was subtracted from all samples. Data was graphed and analyzed on GraphPad Prism (v9.5.1). EC₅₀s were determined via agonist vs response-variable slope (four-parameter) curve fitting and % of enfortumab maximum binding calculated by setting the average of maximum enfortumab MFIs for each cell line at 100%.

The nine nectin-4 antibodies did not bind nectin-4 negative T24 parental line. As shown in Table 5, the antibodies showed good binding to both high and low nectin-4 expression tumor cells.

TABLE 5
Cell Surface Binding of Nectin-4 Antibodies to Nectin-4 expressing
cell lines

| | Average Max | | % of enfortumab | | | |
|---|---|---|---|---|---|---|
| | MFI | | (Average Max MFI) | | EC₅₀ (nM) | |
| | NCI- | SUM- | NCI- | SUM- | NCI- | SUM- |
| mAbs | H1781 | 190PT | H1781 | 190PT | H1781 | 190PT |
| Enfortumab | 9.1E+04 | 1.1E+06 | 100.0% | 100.0% | 0.05 | 0.11 |
| Ab1 | 7.3E+04 | 8.4E+05 | 80.8% | 79.3% | 0.92 | 0.55 |
| Ab1a | 7.7E+04 | 5.9E+05 | 84.5% | 55.6% | 0.20 | 0.12 |
| Ab2 | 8.7E+04 | 9.2E+05 | 95.7% | 87.2% | 0.06 | 0.19 |
| Ab3 | 9.6E+04 | 1.2E+06 | 105.4% | 111.8% | 0.10 | 0.23 |
| Ab4 | 8.7E+04 | 1.0E+06 | 95.8% | 97.9% | 0.14 | 0.19 |
| Ab5 | 9.3E+04 | 1.5E+06 | 102.2% | 144.8% | 0.08 | 0.31 |
| Ab6 | 8.5E+04 | 1.3E+06 | 93.8% | 126.9% | 0.03 | 0.17 |
| Ab7 | 8.7E+04 | 1.2E+06 | 96.0% | 111.8% | 0.09 | 0.14 |
| Ab8 | 9.0E+04 | 8.7E+06 | 99.6% | 81.9% | 0.10 | 0.11 |

MFI = Median Fluorescence Intensity

### Characterizing the Binding of Nectin-4 Antibodies and ADCs to Normal Human Epidermal Keratinocytes Using Flow Cytometry

To characterize binding of nectin-4 antibodies and ADCs of the present disclosure to normal human epidermal keratinocytes, HEKα cells were plated in 150×25 mm tissue culture dish (Corning Inc.) in complete culture medium (Dermal Cell Basal Medium+Keratinocytes Growth Kit, ATCC) at 1 million per well to achieve confluency within 24 hours. The differentiation process progressed for an additional 5-10 days. Following the induction of differentiation, cells were dissociated with 2 mg/mL Type XI Collagenase from Clostridium histolyticum (Sigma-Aldrich) at 37° C. Cells were counted and aliquoted to a V-bottom polypropylene 96-well plate (Thermo Scientific Nunc) at 10⁵ cells/well. Antibodies and ADCs were added to cells starting at 300 nM, 1:4 serially diluted in assay buffer (1×DPBS containing 2% FBS, Gibco). Cells were incubated on a microplate shaker at 4° C., protected from light for 1 hour. After incubation, cells were washed twice with 200 uL/well of assay buffer and stained with Alexa Fluor 647-conjugated AffiniPure F(ab′)2 Fragment Goat Anti-Human IgG (H+L) (Jackson ImmunoResearch) at 1:1,000 dilution. Following an hour incubation at 4° C., cells were washed twice with 200 uL/well of assay buffer and stained with Zombie Green fixable viability dye (BioLegend) at 1:2,000 dilution. Cells were incubated on a microplate shaker at 4° C. for 20 minutes. Cells were washed with assay buffer and fixed with 200 uL/well of BD Cytofix fixation buffer for 20 minutes at 4° C. Cells are resuspended in assay buffer for acquisition on Attune CytPix flow cytometer (Thermo Fisher Scientific). Cells were determined to have antibody binding capacity of 17,000 (using MESF quantification kit, Bangs Laboratories).

As shown in Table 6, the tested nectin-4 antibodies and ADCs (conjugated as in Formula XI, and with a DAR of 8) bind to normal human epidermal keratinocytes at similar or higher EC₅₀ than enfortumab and enfortumab ADC (exemplified herein with each Ab conjugated as in Formula XI, and with a DAR of 8). Ab1 has a lower affinity for HEKα cells than enfortumab.

TABLE 6
Cell Surface Binding to Normal Human Epidermal Keratinocytes
of Nectin-4 Lead Antibodies and CAMP98 ADCs

| mAb | EC₅₀ (nM) | ADC | EC₅₀ (nM) |
|---|---|---|---|
| Enfortumab | 0.06 | Enfortumab | 0.03 |
| Ab1 | 0.32 | Ab1 | 1.6 |
| Ab2 | 0.05 | Ab2 | 0.05 |

EC₅₀ = Half Maximal Effective Concentration

### Example 4: ADC Binding and Internalization

### Characterizing the Internalization Capacity of Nectin-4 Antibodies and ADCs in Human Nectin-4 Positive Cells Using Fluorescence Imaging

T24 cell line was engineered to express human Nectin-4-eGFP and clonally selected for high expression. T24-human Nectin-4-eGFP clone 3 was determined to have antibody binding capacity of 379,000 (using MESF quantification kit, Bangs Laboratories). 124 human Nectin-4-eGFP clone 3 cells were plated at 12,000 cells per well, in black clear-bottom CellCarrier Ultra 384-well microplates (Perkin Elmer) in complete culture medium (McCoy's 5A, modified, +10% FBS+Glutamax+400 μg/mL G418+Pen/Strep). Plates were covered with AeraSeal™ Sealing Films and incubated overnight at 37° C., 5% CO₂. Next day, antibodies and ADCs were added to cells, starting at 300 nM at 1:3 dilution. Plates were covered with AeraSeal™ and placed in incubator for imaging on PerkinElmer Opera Phenix Screening System over a 24 h time course. Data were processed and analyzed in Harmony and Microsoft Excel and graphed in GraphPad Prism.

As shown in Table 7, nectin-4 antibodies and nectin-4 ADCs (exemplified herein with each of Abs1-8 conjugated as in Formula XI, and with a DAR of 8) induce degradation of nectin-4-eGFP signal. The tested nectin-4 ADCs, as shown in Table 7, had internalizing potential equal or greater than the enfortumab antibody in the same ADC format as Abs1-8.

TABLE 7
Percent activity and EC₅₀ (nM) of Nectin-4 Antibodies and ADCs

| | | EC₅₀ | | | EC₅₀ |
|---|---|---|---|---|---|
| mAb | % activity | (nM) | ADC | % activity | (nM) |
| Enfortumab | 97 | 2.13 | Enfortumab | 119.9 | 2.55 |
| Ab1 | 138.7 | 2.08 | Ab1 | 147.4 | 2.02 |
| Ab1a | 144.8 | 2.54 | Ab1a | 150.2 | 2.30 |
| Ab2 | 112.6 | 1.43 | Ab2 | 131.5 | 1.96 |
| Ab3 | 64.4 | 1.30 | Ab3 | 135.5 | 2.68 |
| Ab4 | 40.9 | 0.41 | Ab4 | 142.8 | 2.10 |
| Ab5 | 40.1 | 2.69 | Ab5 | 112.0 | 2.80 |
| Ab6 | 65.1 | 41.42 | Ab6 | 118.0 | 2.26 |
| Ab7 | 66.5 | 3.37 | Ab7 | 126.4 | 2.61 |
| Ab8 | 53.7 | 3.68 | Ab8 | 125.1 | 2.48 |

% activity = max GFP signal loss compared to enfortumab mAb (100%)

### Example 5: ADC Cytotoxicity and Bystander Activity

### Characterizing Cytotoxicity of Nectin-4 ADCs in Low and High Nectin-4 Expression Cell Lines

NCI-H1781 cells, a low expression cell line, were seeded in white clear-bottom 96-well tissue culture plates in culture medium (RPMI 1640+1× GlutaMax+10% Heat Inactivated Fetal Bovine Serum+1 mM sodium pyruvate). Cells were incubated at 37° C. with 5% CO₂ overnight. Next day, ADCs were added at final working concentration from 100 nM, 1:3 serially diluted in culture medium. Plates were covered with Breathe-Easy® sealing membrane and incubated at 37° C. with 5% CO₂. Plates were read after 5 days treatment using CellTiter-Glo Luminescent Cell Viability Assay. 100 ul/well CellTiter-Glo reagent were incubated in plates at room temperature for 10 min. Luminescence was read in the SpectraMax M5e. RLUs (Relative Light Units) were obtained with SoftMax Pro 5.4. Percentage of cell killing was calculated against no treatment as 0%. Data was graphed and analyzed using GraphPad Prism version 9.5.1. IC₅₀ was determined via log(inhibitor) vs. response-variable slope (four parameters) curve fitting.

T24 cell line was engineered to express human nectin-4 and clonally selected. T24-human nectin-4 clone 108, a high expression clonal cell line, was seeded in culture medium (McCoy's 5A+1× GlutaMax+10% Heat Inactivated Fetal Bovine Serum+400 μg/ml G418). ADCs were added at final working concentration from 200 nM, 1:4 serially diluted in culture medium for 5 days.

T24-human Nectin-4 clone 108 and NCI H1781 cells were determined to have antibody binding capacity of 56,000 and 20,000 respectively (using MESF quantification kit, Bangs Laboratories).

As shown in Table 8, select exemplified nectin-4 ADCs (exemplified herein with each of Abs1-8 conjugated as in Formula XI, and with a DAR of 8) exhibited similar robust maximal cell killing as the enfortumab antibody in the same ADC format as Abs1-8 (enfortumab with effector null mutations and conjugated to Formula XI, and with a DAR of 8) on nectin-4 cell lines with differential expression levels. IC₅₀ values suggest similar potencies across the tested ADCs in T24-human nectin-4 clone 108 cells and variable potencies amongst ADCs tested in NCI H1781 cells. No non-specific cytotoxicity was observed on T24 Nectin-4 negative cells tested.

TABLE 8
Cytotoxicity of Nectin-4 conjugates on high
and low expression Nectin-4 cell lines

| | T24 Nectin-4 clone 108 | NCI H1781 |
|---|---|---|
| ADC | (IC50; nM) | (IC50; nM) |
| Ab1 | 0.0837 | 0.2201 |
| Ab1a | 0.0729 | 0.0981 |
| Ab2 | 0.0700 | 0.0473 |
| Ab3 | 0.0825 | 0.0553 |
| Ab4 | 0.1154 | 0.3107 |
| Ab5 | 0.0994 | 0.0657 |
| Ab6 | 0.0783 | 0.0608 |
| Ab7 | 0.0801 | 0.0543 |
| Ab8 | 0.0880 | 0.0572 |
| Enfortumab | 0.0747 | 0.0409 |

### Characterizing Cytotoxicity of Nectin-4 ADCs in MMAE-Resistant Cell Lines

In T24 nectin-4 cells that are resistant to MMAE, two nectin-4 ADCs described herein and enfortumab vedotin were tested for activity. T24-hNectin-4 clone 14 cells were seeded at 500 per well in 100 ul culture medium, in white clear-bottom 96-well tissue culture plates and incubated at 37° C. with 5% CO₂ overnight. The next day each ADC was serially diluted 1:4, with a starting concentration of 400 nM, in culture medium. One-hundred ul of each ADC dilution was added per well and the plates were covered with a Breathe-Easy® sealing membrane and incubated at 37° C. with 5% CO₂. After 5 days of treatment, the plates were removed from the incubator, placed at room temperature for 15 minutes, and 100 ul medium per well was removed. One hundred ul of CellTiter-Glo reagent was added per well and the plates were incubated at room temperature for 10 minutes. RLUs (Relative Light Units) were obtained by SpectraMax M5e with SoftMax Pro 5.4. Percentage of cell killing was calculated against DMSO only (for free payloads) or no treatment (for ADCs) as 0%. Data was graphed and analyzed using Graphpad Prism version 9.5.1. IC50s were determined via log(inhibitor) vs. response-variable slope (four parameters) curve fitting.

ADCs made with Ab1 and Ab2 (conjugated with Formula XI, and with a DAR of 8) retained activity against the MMAE-resistant cells, while enfortumab vedotin lost efficacy.

Bystander Activity of Enfortumab with Exemplified Linker/Payload Compared to Enfortumab Vedotin

UMUC3 cell line was engineered to express human Nectin4 and two clonal populations with different levels of expression were selected. In flat white clear-bottom 96-well plates, UMUC3-hNectin4 clone F7/UMUC3-Luc-GFP cells were mixed and seeded at a total of 1500 cells/well/100 ul with a ratio of 4:1 in assay medium (MEM+1× GlutaMax+10% Heat Inactivated Fetal Bovine Serum+1 mM sodium pyruvate). UMUC3-hNectin4 clone E3/UMUC3-Luc-GFP cells were mixed and seeded at a total of 2100 cells/well/100 ul with a ratio of 6:1 in assay medium. UMUC3-Luc-GFP cells were negative for nectin-4 expression. Plates were incubated at 37° C. with 5% CO₂ overnight. Next day, ADCs were added at final working concentration from 100 nM, 1:3 serially diluted in assay medium. To assess sensitivity of cell line to free payload, free payloads were added at final working concentration from 200 nM, 1:3 serially diluted in assay medium. Plates were covered with Breathe-Easy® sealing membrane and incubated at 37° C. with 5% CO₂. Plates were read after 5-days treatment using ONE-Glo™ Luciferase Assay System. 100 ul/well ONE-Glo™ Assay reagent was incubated in plates at room temperature for 10 min. Luminescence was read in the SpectraMax M5e. RLUs (Relative Light Units) were obtained with SoftMax Pro 5.4. Percentage of UMUC3-Luc-GFP killing was calculated against no treatment as 0%. Data was graphed and analyzed using Graphpad Prism version 9.5.1. IC₅₀ was determined via log(inhibitor) vs. response (three parameters) curve fitting for bystander activity and log(inhibitor) vs. response (four parameters) for free payload cytotoxicity.

UMUC3 Nectin-4 Clone F7 and UMUC3 Nectin-4 Clone E3 cells were determined to have antibody binding capacity of 113,000 and 567,000 respectively (using MESF quantification kit, Bangs Laboratories).

As shown in Table 9, enfortumab conjugated as in Formula XI and with a DAR of 8 showed higher potency of bystander effect on UMUC3-Luc-GFP cells compared to enfortumab vedotin. Notably, Table 10 shows that UMUC3-Luc-GFP cell line was similarly sensitive to free payloads Formula XI and MMAE. Higher expression on positive cell lines results in higher bystander activity by enfortumab vedotin, however, its potency remains lower than enfortumab conjugated as in Formula XI.

TABLE 9
IC50s of Bystander Effect by Enfortumab conjugated
as Formula XI and enfortumab vedotin on UMUC3-
Luc-GFP Nectin-4 negative cells

| | IC₅₀ (nM) | |
|---|---|---|
| | UMUC3-Luc-GFP + | UMUC3-Luc-GFP + |
| | UMUC3- | UMUC3- |
| ADCs | hNectin4 clone F7 | hNectin4 clone E3 |
| Enfortumab conjugated | 0.05 | 0.06 |
| as in Formula XI | | |
| Enfortumab vedotin | 4.30 | 0.35 |

TABLE 10
IC50s of Cytotoxicity by Formula XI payload and MMAE
payload on UMUC3-Luc-GFP Nectin-4 negative cells

| | IC₅₀(nM) |
|---|---|
| ADCs | UMUC3-Luc-GFP |
| Formula XI payload | 0.24 |
| MMAE | 0.38 |

In experiments run as essentially described herein, the bystander effects of four nectin-4 ADCs described herein and enfortumab vedotin were evaluated using the UMUC3-hNectin-4 cloneF7/UMUC3-Luciferase-GFP cell pair. The cells were plated at a ratio of 4:1 and treated with serial dilutions of each ADC for 5 days. To determine the bystander effect of the ADCs, Luciferase luminescence was monitored using ONE-Glo™ Luciferase Assay System. ADCs made with Abs 1, 1a, 2, and 7 (conjugated with Formula XI, and with a DAR of 8) demonstrated potent bystander effects, while enfortumab vedotin showed much less potent bystander effect.

### Characterizing Bystander Effect of Nectin-4 ADCs in Cell Line T24 Nectin-4 Negative Cells

T24 cell line was engineered to express mScarlet and clonally selected to test in bystander assay. T24-mScarlet clone5 and T24-human nectin4 clone108 were mixed and seeded at a total of 1000 cells/well/100 ul with a ratio of 9:1 in culture medium (McCoy's 5A+1× GlutaMax+10% Heat Inactivated Fetal Bovine Serum+400 μg/ml G418) in flat clear bottom 96-well plates. T24 mScarlet clone 5 cells were negative for nectin-4 expression. Plates were incubated at 37° C. with 5% CO₂ overnight. Next day, ADCs were added at final working concentration from 50 nM, 1:4 serially diluted in culture medium. Plates were covered with Breathe-Easy® sealing membrane, placed in BioSpa and scanned in Cytation5 every day for 5 days.

Images of T24-mScarlet cells were captured and analyzed by Cytation5 with Gen5 Image Prime 3.11. Percentage of T24-mScarlet cell killing was calculated by decreased Integrated Intensity (area×mean intensity), normalized to zero hour and no treatment. Data was graphed and analyzed using GraphPad Prism version 9.5.1. IC₅₀ was determined via log(inhibitor) vs. response-variable slope (four parameters) curve fitting.

As shown in Table 11, certain nectin-4 ADCs of this disclosure (exemplified herein with each of Abs1-8 conjugated as in Formula XI, and with a DAR of 8) showed similar potency and magnitude of bystander effect to the enfortumab antibody in the same ADC format as Abs1-8 (enfortumab conjugated with Formula XI, and with a DAR of 8) on T24 mScarlet clone 5 nectin-4 negative cells.

TABLE 11
IC50s of Bystander Effect by Nectin-4 ADC conjugates
on T24 mScarlet clone 5 Nectin-4 negative cells

| ADC | IC50 (nM) |
|---|---|
| Enfortumab | 0.0593 |
| Ab1 | 0.0572 |
| Ab1a | 0.0410 |
| Ab2 | 0.0627 |
| Ab3 | 0.0470 |
| Ab4 | 0.0798 |
| Ab5 | 0.0766 |
| Ab6 | 0.0587 |
| Ab7 | 0.0836 |
| Ab8 | 0.0694 |

Characterizing the Activity of Nectin-4 ADCs with Other Payloads and Other Linker Chemistry in Low and High Nectin-4 Expression Cell Lines

NCI-H1781 cells were seeded in white clear-bottom 96-well tissue culture plates in culture medium (RPMI 1640+1× GlutaMax+10% Heat Inactivated Fetal Bovine Serum+1 mM sodium pyruvate). Cells were incubated at 37° C. with 5% CO₂ overnight. Next day, ADCs were added at final working concentration from 100 nM, 1:4 serially diluted in culture medium. Plates were covered with Breathe-Easy® sealing membrane and incubated at 37° C. with 5% CO₂. Plates were read after 5 days treatment using CellTiter-Glo Luminescent Cell Viability Assay. 100 ul/well CellTiter-Glo reagent were incubated in plates at room temperature for 10 min. Luminescence was read in the SpectraMax M5e. RLUs (Relative Light Units) were obtained with SoftMax Pro 5.4. Percentage of cell killing was calculated against no treatment as 0%. Data was graphed and analyzed using GraphPad Prism version 9.5.1. IC₅₀ was determined via log(inhibitor) vs. response-variable slope (four parameters) curve fitting.

UMUC3 cell line was engineered to express human Nectin4 and clonally selected for high expression. UMUC3-human nectin4 cloneF7 engineered cell line was seeded in culture medium (MEM+1× GlutaMax+10% Heat Inactivated Fetal Bovine Serum+1 mM sodium pyruvate+500 μg/ml G418). ADCs were added at final working concentration from 100 nM, 1:4 serially diluted in culture medium for 6 days.

NCI H1781 and UMUC3-hNectin4 cloneF7 and cells were determined to have antibody binding capacity of 20,000 and 113,000 respectively (using MESF quantification kit, Bangs Laboratories).

As shown in Table 12, both Ab2 ADC conjugated as in Formula XI, and with a DAR of 8 and Ab2 ADC in a PEG8-VA-Exatecan, DAR 8 format exhibit potent cytotoxicity effect on NCI-H1781 and UMUC3-human nectin-4 cloneF7 cell lines.

TABLE 12
Cytotoxicity of Nectin-4 ADCs with other payloads and other linker
chemistry on low and high expression Nectin-4 cell lines

| | | UMUC3 |
|---|---|---|
| | NCI H1781 | Nectin-4 clone F7 |
| ADC | (IC50; nM) | (IC50; nM) |
| Ab2 in Formula XI format | 0.0245 | 0.0569 |
| Ab2 in PEG8-VA-exatecan format | 0.0757 | 0.1125 |
| Negative control ADC | 23.2300 | 16.9800 |

### Example 6: ADCC, ADCP, and/or CDC Assays

### In Vitro Antibody-Dependent Cell-Mediated Cytotoxicity (ADCC) Assay for Nectin-4 Antibodies

T24 cell line was engineered to express human Nectin4 and clonally selected for high expression. T24-human Nectin4 clone 147 was determined to have antibody binding capacity of 411,000 (using MESF quantification kit, Bangs Laboratories). Target cells, T24-human nectin4 clone147 were added in clear tissue culture 96-well plates in test medium (IMDM+1× GlutaMax+10% Heat Inactivated Fetal Bovine Serum+Pen-Strep 100 U/ml-100 μg/ml) and incubated at 37° C. with 5% CO₂ overnight. Next day, 40 ul/well antibodies were added at final working concentration from 200 nM, 1:4 serially diluted in test medium. Antibodies were incubated at 37° C. with 5% CO₂ for one hour. Effector cells, Jurkat-Lucia NFAT-CD16, were then added at 200 k/80 ul/well and incubated at 37° C. with 5% CO₂. 23 h later, 20 ul supernatants and 50 ul of pre-prepared QUANTI-Luc/well were mixed in white opaque plates. Luminescence was read in the SpectraMax M5e. RLUs (Relative Light Units) were obtained with SoftMax Pro 5.4 and plotted on Y-axis against compound concentrations on X-axis using GraphPad Prism version 9.5.1.

Unlike enfortumab antibody, which has wildtype IgG1 Fc, the Abs1-8 are effector null antibodies and did not result in antibody dependent cellular cytotoxicity potential.

### In Vitro Antibody-Dependent Cellular Phagocytosis (ADCP) Assay for Nectin-4 Antibodies

T24 cell line was engineered to express high level of human Nectin4-eGFP and clonally selected. Target cells, T24-human nectin4 eGFP clone3 were seeded in clear tissue culture 96-well plates in test medium (IMDM+1× GlutaMax+10% Heat Inactivated Fetal Bovine Serum+Pen-Strep 100 U/ml-100 μg/ml) and incubated at 37° C. with 5% CO₂ overnight. Next day, 40 ul/well mAbs were added at final working concentration from 200 nM, 1:4 serially diluted in test medium and incubated at 37° C. with 5% CO₂ for 1 h. Effector cells, Jurkat-Lucia NFAT-CD32, were then added at 200 k/80 ul/well and incubated at 37° C. with 5% CO₂. 23 h later, 20 ul supernatants and 50 ul of pre-prepared QUANTI-Luc/well were mixed in white opaque plates. Luminescence was read in the SpectraMax M5e. RLUs (Relative Light Units) were obtained with SoftMax Pro 5.4 and plotted on Y-axis against compound concentrations on X-axis using GraphPad Prism version 9.5.1.

Unlike enfortumab antibody, which has wildtype IgG1 Fc, the Abs1-8 are effector null antibodies and did not result in antibody dependent cellular phagocytosis potential.

### In Vitro Complement-Dependent Cytotoxicity (CDC) Assay for Nectin-4 Lead Antibodies

T24 cell line was engineered to express high level of human Nectin4 and clonally selected. T24-human nectin4 clone147 cells were added in white clear-bottom 96-well tissue culture plates in assay medium (McCoy's 5A+1× GlutaMax+10% Heat Inactivated Fetal Bovine Serum) and incubated in the incubator at 37° C. with 5% CO₂ overnight. Next day, 50 ul/well antibodies were added at final working concentration from 200 nM, 1:3 serially diluted in assay medium and incubated at 37° C. with 5% CO₂ for one hour. Diluted human serum complement (1:3) was then added at 50 ul/well in assay medium and incubated at 37° C. with 5% CO₂ for 3 hours. Plate was read using CellTiter-Glo Luminescent Cell Viability Assay. 100 ul/well CellTiter-Glo reagent were incubated in plates at room temperature for 10 min. Luminescence was read in the SpectraMax M5e. RLUs (Relative Light Units) were obtained with SoftMax Pro 5.4. Percentage of cell killing was calculated against no treatment. Data was graphed and analyzed using GraphPad Prism version 9.5.1.

As a positive control, Jeko-1 cells were treated as above with anti-CD20 antibody in assay medium (RPMI1640+1× GlutaMax+10% Heat Inactivated Fetal Bovine Serum).

Like enfortumab, Abs1-8 did not show CDC potential in nectin-4 expressing T24 cell line. Anti-CD20 control antibody resulted in CDC activity, used as positive control on Jeko-1 cells expressing CD20.

### Example 7: Efficacy of Nectin-4 ADCs in Tumor Xenograft Models

In order to test the efficacy of the nectin-4 ADCs of the present disclosure, which have effector null mutations in the Fc region, and to compare to the enfortumab antibody, which has wildtype Fc and in an ADC with the same payload background as the ones of this disclosure, two tumor xenograft models with either high or moderate nectin-4 expression were tested as described. Immune compromised female mice (nu/nu) between 5-8 weeks of age with weight 18-20 grams were implanted unilaterally on the right flank with UM-UC-3 Nectin4 clone F7 cells with high nectin-4 expression. When tumors reached approximately 150-250 mm³; animals were matched by tumor volume into treatment or control groups and dosing initiated (Day 0, n=8 per group). The test articles were given at a single dose (2 mg/kg) formulated with 5% dextrose. Tumors were measured biweekly until Day 40.

In a separate study, female NSG mice between 5-6 weeks of age were injected subcutaneously with MDA-MB-468 cells with moderate Nectin-4 expression. When tumors reached approximately 150-250 mm³; animals were matched by tumor volume into treatment or control groups and dosing initiated (Day 0, n=8 per group). The test articles were given at a single dose (2 mg/kg) formulated with 5% Dextrose. Tumors were measured biweekly until Day 71.

In both models, a 2 mg/kg single dose treatment of either the nectin-4 ADCs (exemplified herein with each of Abs1-8 conjugated as in Formula XI, and with a DAR of 8) or benchmark enfortumab ADC (enfortumab conjugated with Formula XI, and with a DAR of 8) was used. Tumor growth was measured at day 40 for UMUC3 Nectin-4 clone F7 and day 71 for MDAMB468 xenografts. As shown in Table 13, treatment with the nectin-4 ADCs using effector null Abs1-8 resulted in tumor growth inhibition that was equal or, with certain ADCs, better efficacy than the benchmark enfortumab ADC with a wild-type Fc region.

TABLE 13
Tumor measurements (Mean tumor volume ±
SEM) after treatment with Nectin-4 ADCs

| | UMUC3 Nectin-4 | | MDAMB468 | |
|---|---|---|---|---|
| | clone F7 Xenograft | | Xenograft | |
| ADC | Mean (mm³) | SEM | Mean (mm³) | SEM |
| Vehicle | 1338.34 | 51.2 | 464.25 | 28.43 |
| Enfortumab ADC | 812.22 | 187.34 | 272.28 | 22.15 |
| Control ADC | 1237.24 | 95.92 | 484.75 | 41.46 |
| Ab1 ADC | 192.39 | 69.04 | 121.05 | 12.02 |
| Ab1a ADC | 234.79 | 74.12 | 135.24 | 19.3 |
| Ab2 ADC | 72.57 | 14.06 | 124.95 | 10.32 |
| Ab3 ADC | 993.45 | 189.2 | 366.45 | 31.56 |
| Ab4 ADC | 928.09 | 103.94 | 343.65 | 36.11 |
| Ab5 ADC | 632.99 | 70.34 | 220.38 | 20.41 |
| Ab6 ADC | 866.94 | 119.56 | 304.9 | 27.25 |
| Ab7 ADC | 319.79 | 89.97 | 245.33 | 16.58 |
| Ab8 ADC | 820.06 | 131.53 | 349.91 | 38.36 |

SEM = Standard error of mean

In a xenograft study run similarly as described using the UM-UC-3 Nectin4 clone F7 cells, a nectin-4 ADC described herein was dosed at 0.5 mg/kg, 1 mg/kg, and 2 mg/kg in combination with cisplatin and gemcitabine. An increase in antitumor activity was observed upon combination of SOC agents with the nectin-4 ADC at low doses of 0.5 and 1 mg/kg.

### Amino Acid and Nucleotide Sequences

SEQ ID NO: 1
(human nectin-4)
MPLSLGAEMWGPEAWLLLLLLLASFTGRCPAGELETSDVVTVVLGQDAKLPCFY
RGDSGEQVGQVAWARVDAGEGAQELALLHSKYGLHVSPAYEGRVEQPPPPRNP
LDGSVLLRNAVQADEGEYECRVSTFPAGSFQARLRLRVLVPPLPSLNPGPALEEG
QGLTLAASCTAEGSPAPSVTWDTEVKGTTSSRSFKHSRSAAVTSEFHLVPSRSMN
GQPLTCVVSHPGLLQDQRITHILHVSFLAEASVRGLEDQNLWHIGREGAMLKCLS
EGQPPPSYNWTRLDGPLPSGVRVDGDTLGFPPLTTEHSGIYVCHVSNEFSSRDSQ
VTVDVLDPQEDSGKQVDLVSASVVVVGVIAALLFCLLVVVVVLMSRYHRRKAQ
QMTQKYEEELTLTRENSIRRLHSHHTDPRSQPEESVGLRAEGHPDSLKDNSSCSV
MSEEPEGRSYSTLTTVREIETQTELLSPGSGRAEEEEDQDEGIKQAMNHFVQENG
TLRAKPTGNGIYINGRGHLV
SEQ ID NO: 2
(HC of Ab1)
QVQLVQSGAEVKKPGASVKVSCKASGYTFTGYYIHWVRQAPGQGLEWMGWIN
PNSGGTNYAQKFQGRVTMTRDTSISTAYMELSRLRSDDTAVYYCAREDWDFDY
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVE
PKSCDKTHTCPPCPAPEAAGGPSVFLFPPKPKDTLMISRTPEVTCVVVSVSHEDPE
VKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALH
NHYTQKSLSLSPGK
SEQ ID NO: 3
(LC of Ab1)
EIVLTQSPGTLSLSPGERATLSCRTSQSVSSSYLAWYQQKPGQAPRLLIYGASNRA
TGIPDRFSGSGSGTDFTLTISRLEPEDFAVYYCQQYGSSPITFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 4
(HCDR1 of Ab1 and 1a)
KASGYTFTGYYIH
SEQ ID NO: 5
(HCDR2 of Ab1 and 1a)
WINPNSGGTN
SEQ ID NO: 6
(HCDR3 of Ab1 and 1a)
AREDWDFDY
SEQ ID NO: 7
(LCDR1 of Ab1 and 1a)
RTSQSVSSSYLA
SEQ ID NO: 8
(LCDR2 of Ab1 and 1a)
YGASNRAT
SEQ ID NO: 9
(LCDR3 of Ab1 and 1a)
QQYGSSPIT
SEQ ID NO: 10
(HCVR of Ab1)
QVQLVQSGAEVKKPGASVKVSCKASGYTFTGYYIHWVRQAPGQGLEWMGWIN
PNSGGTNYAQKFQGRVTMTRDTSISTAYMELSRLRSDDTAVYYCAREDWDFDY
WGQGTLVTVSS
SEQ ID NO: 11
(LCVR of Ab1)
EIVLTQSPGTLSLSPGERATLSCRTSQSVSSSYLAWYQQKPGQAPRLLIYGASNRA
TGIPDRFSGSGSGTDFTLTISRLEPEDFAVYYCQQYGSSPITFGQGTKVEIK
SEQ ID NO: 12
(HC of Ab1a)
QVQLVQSGAQVKKPGASVKVSCKASGYTFTGYYIHWVRQAPGQGLEWMGWIN
PNSGGTNYAQKFQGRVTMTRDTSISTAYMELSRLRSDDTAVYFCAREDWDFDY
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVE
PKSCDKTHTCPPCPAPEAAGGPSVFLFPPKPKDTLMISRTPEVTCVVVSVSHEDPE
VKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALH
NHYTQKSLSLSPGK
SEQ ID NO: 13
(LC of Ab1a)
EIVLTQSPGTLSLSPGERATLSCRTSQSVSSSYLAWYQQKPGQAPRLLIYGASNRA
TDIPDRFSGSGSGTDFTLTINRLEPEDFAVYYCQQYGSSPITFGQGTRLEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 14
(HCVR of Ab1a)
QVQLVQSGAQVKKPGASVKVSCKASGYTFTGYYIHWVRQAPGQGLEWMGWIN
PNSGGTNYAQKFQGRVTMTRDTSISTAYMELSRLRSDDTAVYFCAREDWDFDY
WGQGTLVTVSS
SEQ ID NO: 15
(LCVR of Ab1a)
EIVLTQSPGTLSLSPGERATLSCRTSQSVSSSYLAWYQQKPGQAPRLLIYGASNRA
TDIPDRFSGSGSGTDFTLTINRLEPEDFAVYYCQQYGSSPITFGQGTRLEIK
SEQ ID NO: 16
(HC of Ab2)
EVQLVESGGGLVQPGGSLRLSCAASGFTFSSYDMHWVRQATGKGLEWVSAIGT
VGDTYYPGSVKGRFTISRENAKNSLYLQMNSLRAGDTAVYYCAREWNGMDVW
GQGTTVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEP
KSCDKTHTCPPCPAPEAAGGPSVFLFPPKPKDTLMISRTPEVTCVVVSVSHEDPEV
KFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNH
YTQKSLSLSPGK
SEQ ID NO: 17
(LC of Ab2)
DIVMTQSPDSLAVSLGERATINCKSSQSVLYNSNNKNYLAWYQQKPGQPPKLLIY
WASTRESGVPDRFSGSGSGTDFTLTISSLQAEDVAVYYCQQFYTTPYSFGQGTKV
EIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
SEQ ID NO: 18
(HCDR1 of Ab2)
AASGFTFSSYDMH
SEQ ID NO: 19
(HCDR2 of Ab2)
AIGTVGDTY
SEQ ID NO: 20
(HCDR3 of Ab2)
AREWNGMDV
SEQ ID NO: 21
(LCDR1 of Ab2)
KSSQSVLYNSNNKNYLA
SEQ ID NO: 22
(LCDR2 of Ab2)
YWASTRES
SEQ ID NO: 23
(LCDR3 of Ab2)
QQFYTTPYS
SEQ ID NO: 24
(HCVR of Ab2)
EVQLVESGGGLVQPGGSLRLSCAASGFTFSSYDMHWVRQATGKGLEWVSAIGT
VGDTYYPGSVKGRFTISRENAKNSLYLQMNSLRAGDTAVYYCAREWNGMDVW
GQGTTVTVSS
SEQ ID NO: 25
(LCVR of Ab2)
DIVMTQSPDSLAVSLGERATINCKSSQSVLYNSNNKNYLAWYQQKPGQPPKLLIY
WASTRESGVPDRFSGSGSGTDFTLTISSLQAEDVAVYYCQQFYTTPYSFGQGTKV
EIK
SEQ ID NO: 26
(HC of Ab3)
EVQLVESGGGLVKPGGSLRLSCAASGFNLNHYNMNWVRQAPGKGLEWVSSVSS
GGGFRYYADSVRGRFTISRDNAKNSLYLQMNSLRAEDTAVYYCARGAVFHDAF
DIWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKR
VEPKSCDKTHTCPPCPAPEAAGGPSVFLFPPKPKDTLMISRTPEVTCVVVSVSHED
PEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEA
LHNHYTQKSLSLSPGK
SEQ ID NO: 27
(LC of Ab3)
DIQLTQSPSFLSASVGDRVTITCRASQDISSYLAWYQQKPGKAPKLLIYVASTLQS
GVPSRFSGSGSGTEFTLTISSLQPEDFATYYCQQINSYPFTFGQGTKVEIKRTVAAP
SVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 28
(HCDR1 of Ab3)
AASGFNLNHYNMN
SEQ ID NO: 29
(HCDR2 of Ab3)
SVSSGGGFRY
SEQ ID NO: 30
(HCDR3 of Ab3)
ARGAVFHDAFDI
SEQ ID NO: 31
(LCDR1 of Ab3)
RASQDISSYLA
SEQ ID NO: 32
(LCDR2 of Ab3)
YVASTLQS
SEQ ID NO: 33
(LCDR3 of Ab3)
QQINSYPFT
SEQ ID NO: 34
(HCVR of Ab3)
EVQLVESGGGLVKPGGSLRLSCAASGFNLNHYNMNWVRQAPGKGLEWVSSVSS
GGGFRYYADSVRGRFTISRDNAKNSLYLQMNSLRAEDTAVYYCARGAVFHDAF
DIWGQGTLVTVSS
SEQ ID NO: 35
(LCVR of Ab3)
DIQLTQSPSFLSASVGDRVTITCRASQDISSYLAWYQQKPGKAPKLLIYVASTLQS
GVPSRFSGSGSGTEFTLTISSLQPEDFATYYCQQINSYPFTFGQGTKVEIK
SEQ ID NO: 36
(HC of Ab4)
QVQLQESGPGLVKPSETLSLTCTVSGGSISGYYWSWIRQPPGKGLEWIGYIYYSGS
TNYNPSLKSRVTISVDTSKNQFSLKLSSVTAADTAVYYCARLGIFFDAFDIWGQG
TLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSC
DKTHTCPPCPAPEAAGGPSVFLFPPKPKDTLMISRTPEVTCVVVSVSHEDPEVKFN
WYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYT
QKSLSLSPGK
SEQ ID NO: 37
(LC of Ab4)
DIQLTQSPSFLSASVGDRVTITCRASQGISSYLAWYQQKPGKAPKLLIYAASTLOS
GVPSRFSGSGSGTEFTLTISSLQPEDFATYYCQQFNSYPWTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 38
(HCDR1 of Ab4)
TVSGGSISGYYWS
SEQ ID NO: 39
(HCDR2 of Ab4)
YIYYSGSTN
SEQ ID NO: 40
(HCDR3 of Ab4)
ARLGIFFDAFDI
SEQ ID NO: 41
(LCDR1 of Ab4 and Ab5)
RASQGISSYLA
SEQ ID NO: 42
(LCDR2 of Ab4 and Ab5)
YAASTLQS
SEQ ID NO: 43
(LCDR3 of Ab4)
QQFNSYPWT
SEQ ID NO: 44
(HCVR of Ab4)
QVQLQESGPGLVKPSETLSLTCTVSGGSISGYYWSWIRQPPGKGLEWIGYIYYSGS
TNYNPSLKSRVTISVDTSKNQFSLKLSSVTAADTAVYYCARLGIFFDAFDIWGQG
TLVTVSS
SEQ ID NO: 45
(LCVR of Ab4)
DIQLTQSPSFLSASVGDRVTITCRASQGISSYLAWYQQKPGKAPKLLIYAASTLQS
GVPSRFSGSGSGTEFTLTISSLQPEDFATYYCQQFNSYPWTFGQGTKVEIK
SEQ ID NO: 46
(HC of Ab5)
QVQLVQSGAEVKKPGASVKVSCKASGYTFTSYYVHWVRQAPGQGLEWMGIINP
SIISTSYAQKFQARVTMTRDTSTSTVYMELSSLRSEDTAVYYCARGLNFDAFDIW
GQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEP
KSCDKTHTCPPCPAPEAAGGPSVFLFPPKPKDTLMISRTPEVTCVVVSVSHEDPEV
KFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNH
YTQKSLSLSPGK
SEQ ID NO: 47
(LC of Ab5)
DIQLTQSPSFLSASVGDRVTITCRASQGISSYLAWYQQKPGKAPKLLIYAASTLQS
GVPSRFSGSGSGTEFTLTISSLQPEDFATYYCQQLNNYPFTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 48
(HCDR1 of Ab5)
KASGYTFTSYYVH
SEQ ID NO: 49
(HCDR2 of Ab5)
IINPSIISTS
SEQ ID NO: 50
(HCDR3 of Ab5)
ARGLNFDAFDI
SEQ ID NO: 51
(LCDR3 of Ab5)
QQLNNYPFT
SEQ ID NO: 52
(HCVR of Ab5)
QVQLVQSGAEVKKPGASVKVSCKASGYTFTSYYVHWVRQAPGQGLEWMGIINP
SIISTSYAQKFQARVTMTRDTSTSTVYMELSSLRSEDTAVYYCARGLNFDAFDIW
GQGTLVTVSS
SEQ ID NO: 53
(LCVR of Ab5)
DIQLTQSPSFLSASVGDRVTITCRASQGISSYLAWYQQKPGKAPKLLIYAASTLQS
GVPSRFSGSGSGTEFTLTISSLQPEDFATYYCQQLNNYPFTFGQGTKVEIK
SEQ ID NO: 54
(HC of Ab6)
QVQLVQSGAEVKKPGASVKVSCKASGYTFTGYYMHWVRQAPGQGLEWMGIIN
PSGGSTTYAQKFQGRVTMTRDTSTSTVYMELSSLRSEDTAVYYCARGRLGTYFD
YWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRV
EPKSCDKTHTCPPCPAPEAAGGPSVFLFPPKPKDTLMISRTPEVTCVVVSVSHEDP
EVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEAL
HNHYTQKSLSLSPGK
SEQ ID NO: 55
(LC of Ab6)
SYVLTQPPSVSVAPGQTARITCGGNNIGSKSVHWYQQKPGQAPVLVVYDDSDRP
SGIPERFSGSNSGNTATLTISRVEAGDEADYYCQVWDRSSDQVVFGGGTKLTVLG
QPKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECS
SEQ ID NO: 56
(HCDR1 of Ab6)
KASGYTFTGYYMH
SEQ ID NO: 57
(HCDR2 of Ab6)
IINPSGGSTT
SEQ ID NO: 58
(HCDR3 of Ab6)
ARGRLGTYFDY
SEQ ID NO: 59
(LCDR1 of Ab6)
GGNNIGSKSVH
SEQ ID NO: 60
(LCDR2 of Ab6)
YDDSDRPS
SEQ ID NO: 61
(LCDR3 of Ab6)
QVWDRSSDQVV
SEQ ID NO: 62
(HCVR of Ab6)
QVQLVQSGAEVKKPGASVKVSCKASGYTFTGYYMHWVRQAPGQGLEWMGIIN
PSGGSTTYAQKFQGRVTMTRDTSTSTVYMELSSLRSEDTAVYYCARGRLGTYFD
YWGQGTLVTVSS
SEQ ID NO: 63
(LCVR of Ab6)
SYVLTQPPSVSVAPGQTARITCGGNNIGSKSVHWYQQKPGQAPVLVVYDDSDRP
SGIPERFSGSNSGNTATLTISRVEAGDEADYYCQVWDRSSDQVVFGGGTKLTVL
SEQ ID NO: 64
(HC of Ab7)
QVQLVQSGAEVKKPGASVKVSCKASGYTFTNYYMHWVRQAPGQGLEWMGIIN
PSSGSASYAQKFQGRVTMTRDTSTSTVYMELSSLRSEDTAVYYCAREGVGRDIL
QAFDIWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKRVEPKSCDKTHTCPPCPAPEAAGGPSVFLFPPKPKDTLMISRTPEVTCVVVSVS
HEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVM
HEALHNHYTQKSLSLSPGK
SEQ ID NO: 65
(LC of Ab7)
QSVLTQPPSVSEAPRQRVTISCSGSSSNIGNNAVNWYQQLPGKAPKLLIYYDDLLP
SGVSDRFSGSKSGTSASLAISGLQSEDEADYYCAAWDDSLNGHVFGGGTKLTVL
GQPKAAPSVTLFPPSSEELQANKATLVCLISDFYPGAVTVAWKADSSPVKAGVET
TTPSKQSNNKYAASSYLSLTPEQWKSHRSYSCQVTHEGSTVEKTVAPTECS
SEQ ID NO: 66
(HCDR1 of Ab7)
KASGYTFTNYYMH
SEQ ID NO: 67
(HCDR2 of Ab7)
IINPSSGSAS
SEQ ID NO: 68
(HCDR3 of Ab7)
AREGVGRDILQAFDI
SEQ ID NO: 69
(LCDR1 of Ab7 and Ab8)
SGSSSNIGNNAVN
SEQ ID NO: 70
(LCDR2 of Ab7 and Ab8)
YYDDLLPS
SEQ ID NO: 71
(LCDR3 of Ab7)
AAWDDSLNGHV
SEQ ID NO: 72
(HCVR of Ab7)
QVQLVQSGAEVKKPGASVKVSCKASGYTFTNYYMHWVRQAPGQGLEWMGIIN
PSSGSASYAQKFQGRVTMTRDTSTSTVYMELSSLRSEDTAVYYCAREGVGRDIL
QAFDIWGQGTLVTVSS
SEQ ID NO: 73
(LCVR of Ab7)
QSVLTQPPSVSEAPRQRVTISCSGSSSNIGNNAVNWYQQLPGKAPKLLIYYDDLLP
SGVSDRFSGSKSGTSASLAISGLQSEDEADYYCAAWDDSLNGHVFGGGTKLTVL
SEQ ID NO: 74
(HC of Ab8)
QVQLVQSGAEVKKPGASVKVSCKASGYTFTSYYIHWVRQAPGQGLEWMGIINPI
SGRTSSAQKFQGRVTMTRDTSTSTVYMELSSLRSEDTAVYYCAKEGVGGELLRA
FDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
RVEPKSCDKTHTCPPCPAPEAAGGPSVFLFPPKPKDTLMISRTPEVTCVVVSVSHE
DPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHE
ALHNHYTQKSLSLSPGK
SEQ ID NO: 75
(LC of Ab8)
QSVLTQPPSVSEAPRQRVTISCSGSSSNIGNNAVNWYQQLPGKAPKLLIYYDDLLP
SGVSDRFSGSKSGTSASLAISGLQSEDEADYYCAAWDDSLNGFVFGGGTKLTVL
GQPKAAPSVTLFPPSSEELQANKATLVCLISDFYPGAVTVAWKADSSPVKAGVET
TTPSKQSNNKYAASSYLSLTPEQWKSHRSYSCQVTHEGSTVEKTVAPTECS
SEQ ID NO: 76
(HCDR1 of Ab8)
KASGYTFTSYYIH
SEQ ID NO: 77
(HCDR2 of Ab8)
IINPISGRTS
SEQ ID NO: 78
(HCDR3 of Ab8)
AKEGVGGELLRAFDI
SEQ ID NO: 79
(LCDR3 of Ab8)
AAWDDSLNGFV
SEQ ID NO: 80
(HCVR of Ab8)
QVQLVQSGAEVKKPGASVKVSCKASGYTFTSYYIHWVRQAPGQGLEWMGIINPI
SGRTSSAQKFQGRVTMTRDTSTSTVYMELSSLRSEDTAVYYCAKEGVGGELLRA
FDIWGQGTLVTVSS
SEQ ID NO: 81
(LCVR of Ab8)
QSVLTQPPSVSEAPRQRVTISCSGSSSNIGNNAVNWYQQLPGKAPKLLIYYDDLLP
SGVSDRFSGSKSGTSASLAISGLQSEDEADYYCAAWDDSLNGFVFGGGTKLTVL
SEQ ID NO: 82
(DNA of HC for Ab1)
CAAGTTCAGCTGGTGCAGTCTGGAGCCGAAGTGAAGAAGCCCGGAGCCTCAG
TGAAAGTGTCCTGTAAGGCGTCCGGCTATACCTTCACTGGTTACTATATCCAC
TGGGTACGCCAGGCTCCAGGACAGGGCTTGGAGTGGATGGGTTGGATCAATC
CTAACTCTGGGGGCACCAACTACGCACAGAAATTCCAAGGGAGAGTCACCAT
GACGCGGGATACTAGCATTAGCACAGCTTATATGGAGCTCTCGCGTCTGAGG
AGTGACGATACTGCCGTCTACTACTGCGCACGAGAAGACTGGGACTTTGATT
ATTGGGGGCAGGGCACACTTGTGACAGTTTCAAGTgcctccaccaagggcccatcggtcttcc
ccctggcaccctcctccaagagcacctctgggggcacagcggccctgggctgcctggtcaaggactacttccccgaaccggtg
acggtgtcgtggaactcaggcGCGctgaccagcggcgtgcacaccttcccggctgtcctacagtcctcaggactctactccct
cagcagcgtggtgaccgtgccctccagcagcttgggcacccagacctacatctgcaacgtgaatcacaagcccagcaacacca
aggtggacaagagagttgagcccaaatcttgtgacaaaactcacacatgcccaccgtgcccagcacctgaagccgccggggg
accgtcagtcttcctcttccccccaaaacccaaggacaccctcatgatctcccggacccctgaggtcacatgcgtggtggtgtcc
gtgagccacgaagaccctgaggtcaagttcaactggtatgtggacggcgtggaggtgcataatgccaagacaaagccgcggg
aggagcagtacaacagcacgtaccgtgtggtcagcgtcctcaccgtcctgcaccaagactggctgaatggcaaggagtacaag
tgcaaggtctccaacaaagccctcccagcccccatcgagaaaaccatctccaaagccaaagggcagccccgagaaccacag
gtgtacaccctgcccccatcccgggaggagatgaccaagaaccaagtcagcctgacctgcctggtcaaaggcttctatcccagc
gacatcgccgtggagtgggagagcaatgggcagccggagaacaactacaagaccacgcctcccgtgctggactccgacggc
tccttcttcctctattccaagctcaccgtggacaagagcaggtggcagcaggggaacgtcttctcatgctccgtgatgcatgaggc
tctgcacaaccactacacgcagaagagcctctccctgtctccgggcaaa
SEQ ID NO: 83
(DNA of LC for Ab1)
GAGATCGTGCTGACCCAGAGTCCAGGAACACTCAGCCTGTCCCCCGGAGAAC
GGGCTACTTTGTCATGCCGTACGAGCCAGAGCGTGTCCTCTTCTTATCTGGCT
TGGTACCAGCAAAAGCCTGGACAAGCGCCTCGATTACTTATCTATGGTGCCTC
TAACCGCGCCACAGGCATTCCAGACAGATTCTCAGGGTCTGGCAGTGGCACC
GACTTTACACTAACCATTTCCAGGCTCGAACCCGAGGATTTCGCAGTCTACTA
CTGTCAGCAGTATGGGTCGAGCCCGATTACTTTTGGCCAGGGTACCAAGGTTG
AGATCAAAagaactgtggcggcgccatctgtcttcatcttcccgccatctgatgagcagttgaaatccggaactgcctctg
ttgtgtgcctgctgaataacttctatcccagagaggccaaagtacagtggaaggtggataacgccctccaatcgggtaactccca
ggagagtgtcacagagcaggacagcaaggacagcacctacagcctcagcagcaccctgacgctgagcaaagcagactacga
gaaacacaaagtctacgcctgcgaagtcacccatcagggcctgagctcgcccgtcacaaagagcttcaacaggggagagtgc
SEQ ID NO: 84
(DNA of HC for Ab1a)
CAGGTTCAACTTGTGCAAAGTGGAGCACAGGTTAAGAAGCCTGGCGCATCCG
TGAAAGTCTCTTGCAAAGCATCCGGGTACACATTCACCGGCTACTATATCCAT
TGGGTACGGCAGGCCCCAGGTCAGGGGCTTGAATGGATGGGATGGATAAATC
CCAATAGCGGTGGTACCAATTACGCCCAGAAGTTTCAGGGACGAGTCACAAT
GACTAGAGATACAAGTATCTCAACCGCATACATGGAACTCTCAAGACTGAGA
TCAGATGACACCGCAGTCTATTTCTGTGCTCGCGAGGATTGGGACTTCGATTA
CTGGGGCCAAGGCACCCTGGTTACCGTCAGCAGCgcctccaccaagggcccatcggtcttccc
cctggcaccctcctccaagagcacctctgggggcacagcggccctgggctgcctggtcaaggactacttccccgaaccggtga
cggtgtcgtggaactcaggcGCGctgaccagcggcgtgcacaccttcccggctgtcctacagtcctcaggactctactccctc
agcagcgtggtgaccgtgccctccagcagcttgggcacccagacctacatctgcaacgtgaatcacaagcccagcaacaccaa
ggtggacaagagagttgagcccaaatcttgtgacaaaactcacacatgcccaccgtgcccagcacctgaagccgccggggga
ccgtcagtcttcctcttccccccaaaacccaaggacaccctcatgatctcccggacccctgaggtcacatgcgtggtggtgtccgt
gagccacgaagaccctgaggtcaagttcaactggtatgtggacggcgtggaggtgcataatgccaagacaaagccgcgggag
gagcagtacaacagcacgtaccgtgtggtcagcgtcctcaccgtcctgcaccaagactggctgaatggcaaggagtacaagtg
caaggtctccaacaaagccctcccagcccccatcgagaaaaccatctccaaagccaaagggcagccccgagaaccacaggt
gtacaccctgcccccatcccgggaggagatgaccaagaaccaagtcagcctgacctgcctggtcaaaggcttctatcccagcg
acatcgccgtggagtgggagagcaatgggcagccggagaacaactacaagaccacgcctcccgtgctggactccgacggct
ccttcttcctctattccaagctcaccgtggacaagagcaggtggcagcaggggaacgtcttctcatgctccgtgatgcatgaggct
ctgcacaaccactacacgcagaagagcctctccctgtctccgggcaaa
SEQ ID NO: 85
(DNA of LC for Ab1a)
GAAATCGTGCTTACCCAATCCCCTGGTACTCTCTCCCTCAGCCCCGGCGAACG
GGCCACCCTCTCCTGCCGAACAAGCCAAAGTGTGTCTAGCTCCTACCTCGCCT
GGTATCAACAGAAACCCGGCCAAGCACCACGACTTCTTATCTATGGTGCAAG
CAACAGGGCTACTGACATTCCAGATCGCTTCAGTGGCTCTGGCTCAGGAACA
GACTTCACTCTGACTATCAACAGGTTGGAACCTGAGGATTTTGCCGTATATTA
TTGCCAACAATACGGCTCCTCTCCTATAACCTTTGGTCAAGGTACACGCCTGG
AAATAAAGagaactgtggcggcgccatctgtcttcatcttcccgccatctgatgagcagttgaaatccggaactgcctctg
ttgtgtgcctgctgaataacttctatcccagagaggccaaagtacagtggaaggtggataacgccctccaatcgggtaactccca
ggagagtgtcacagagcaggacagcaaggacagcacctacagcctcagcagcaccctgacgctgagcaaagcagactacga
gaaacacaaagtctacgcctgcgaagtcacccatcagggcctgagctcgcccgtcacaaagagcttcaacaggggagagtgc
SEQ ID NO: 86
(DNA of HC for Ab2)
GAAGTCCAACTAGTGGAGAGCGGCGGGGGCCTGGTCCAGCCCGGGGGTTCTC
TCCGTCTCTCTTGCGCTGCATCCGGATTCACATTCTCATCCTATGATATGCACT
GGGTGCGACAGGCAACGGGGAAGGGCCTGGAGTGGGTTAGCGCCATTGGTA
CTGTGGGCGACACTTACTATCCTGGAAGCGTGAAGGGACGGTTTACAATCTC
AAGGGAGAATGCCAAAAACAGTCTGTACCTTCAGATGAACAGTTTGCGCGCT
GGCGACACCGCCGTTTACTATTGTGCGAGAGAATGGAATGGAATGGATGTGT
GGGGCCAGGGTACCACCGTCACAGTATCCTCTgcctccaccaagggcccatcggtcttccccctg
gcaccctcctccaagagcacctctgggggcacagcggccctgggctgcctggtcaaggactacttccccgaaccggtgacggt
gtcgtggaactcaggcGCGctgaccagcggcgtgcacaccttcccggctgtcctacagtcctcaggactctactccctcagca
gcgtggtgaccgtgccctccagcagcttgggcacccagacctacatctgcaacgtgaatcacaagcccagcaacaccaaggtg
gacaagagagttgagcccaaatcttgtgacaaaactcacacatgcccaccgtgcccagcacctgaagccgccgggggaccgt
cagtcttcctcttccccccaaaacccaaggacaccctcatgatctcccggacccctgaggtcacatgcgtggtggtgtccgtgag
ccacgaagaccctgaggtcaagttcaactggtatgtggacggcgtggaggtgcataatgccaagacaaagccgcgggaggag
cagtacaacagcacgtaccgtgtggtcagcgtcctcaccgtcctgcaccaagactggctgaatggcaaggagtacaagtgcaa
ggtctccaacaaagccctcccagcccccatcgagaaaaccatctccaaagccaaagggcagccccgagaaccacaggtgtac
accctgcccccatcccgggaggagatgaccaagaaccaagtcagcctgacctgcctggtcaaaggcttctatcccagcgacat
cgccgtggagtgggagagcaatgggcagccggagaacaactacaagaccacgcctcccgtgctggactccgacggctccttc
ttcctctattccaagctcaccgtggacaagagcaggtggcagcaggggaacgtcttctcatgctccgtgatgcatgaggctctgc
acaaccactacacgcagaagagcctctccctgtctccgggcaaa
SEQ ID NO: 87
(DNA of LC for Ab2)
GACATTGTCATGACCCAGTCACCTGACTCCTTGGCGGTGAGCCTGGGAGAAA
GAGCAACAATTAACTGCAAATCCAGTCAATCGGTCCTCTACAACTCCAATAA
CAAGAATTACCTGGCTTGGTATCAGCAGAAGCCCGGCCAGCCACCTAAGCTC
CTGATCTATTGGGCTAGTACTCGGGAGTCCGGAGTTCCCGATAGGTTCTCTGG
TTCAGGGTCTGGCACCGACTTTACCCTTACAATAAGCAGCCTACAGGCCGAA
GATGTAGCCGTGTATTACTGTCAACAGTTCTATACAACGCCATACTCTTTTGG
CCAGGGGACTAAAGTGGAGATCAAGagaactgtggcggcgccatctgtcttcatcttcccgccatctgat
gagcagttgaaatccggaactgcctctgttgtgtgcctgctgaataacttctatcccagagaggccaaagtacagtggaaggtgg
ataacgccctccaatcgggtaactcccaggagagtgtcacagagcaggacagcaaggacagcacctacagcctcagcagcac
cctgacgctgagcaaagcagactacgagaaacacaaagtctacgcctgcgaagtcacccatcagggcctgagctcgcccgtc
acaaagagcttcaacaggggagagtgc
SEQ ID NO: 88
(DNA of HC for Ab3)
GAGGTGCAGCTGGTTGAGAGCGGCGGCGGGTTAGTCAAGCCTGGTGGCTCCC
TTCGGCTGTCCTGTGCAGCCTCGGGGTTCAATCTGAACCACTACAACATGAAC
TGGGTGCGTCAAGCCCCCGGCAAGGGACTCGAATGGGTCTCTAGTGTTTCCTC
AGGGGGTGGATTTCGCTACTATGCTGACAGCGTAAGGGGACGATTTACCATT
TCTCGGGATAATGCCAAAAACAGCTTGTACCTGCAGATGAATAGTCTAAGAG
CAGAAGATACCGCTGTGTATTATTGCGCGAGAGGTGCCGTCTTCCATGACGCT
TTCGACATCTGGGGACAGGGCACTCTCGTGACAGTGTCATCTgcctccaccaagggccc
atcggtcttccccctggcaccctcctccaagagcacctctgggggcacagcggccctgggctgcctggtcaaggactacttccc
cgaaccggtgacggtgtcgtggaactcaggcGCGctgaccagcggcgtgcacaccttcccggctgtcctacagtcctcagg
actctactccctcagcagcgtggtgaccgtgccctccagcagcttgggcacccagacctacatctgcaacgtgaatcacaagcc
cagcaacaccaaggtggacaagagagttgagcccaaatcttgtgacaaaactcacacatgcccaccgtgcccagcacctgaag
ccgccgggggaccgtcagtcttcctcttccccccaaaacccaaggacaccctcatgatctcccggacccctgaggtcacatgcg
tggtggtgtccgtgagccacgaagaccctgaggtcaagttcaactggtatgtggacggcgtggaggtgcataatgccaagacaa
agccgcgggaggagcagtacaacagcacgtaccgtgtggtcagcgtcctcaccgtcctgcaccaagactggctgaatggcaa
ggagtacaagtgcaaggtctccaacaaagccctcccagcccccatcgagaaaaccatctccaaagccaaagggcagccccga
gaaccacaggtgtacaccctgcccccatcccgggaggagatgaccaagaaccaagtcagcctgacctgcctggtcaaaggctt
ctatcccagcgacatcgccgtggagtgggagagcaatgggcagccggagaacaactacaagaccacgcctcccgtgctgga
ctccgacggctccttcttcctctattccaagctcaccgtggacaagagcaggtggcagcaggggaacgtcttctcatgctccgtga
tgcatgaggctctgcacaaccactacacgcagaagagcctctccctgtctccgggcaaa
SEQ ID NO: 89
(DNA of LC for Ab3)
GATATCCAACTGACCCAGTCTCCAAGTTTCCTGTCTGCTTCAGTGGGCGATAG
GGTCACAATCACCTGTCGGGCCTCCCAAGACATCTCCTCGTACCTGGCGTGGT
ACCAGCAGAAGCCTGGCAAGGCTCCCAAATTGCTCATATACGTTGCAAGTAC
CCTTCAGAGCGGAGTGCCATCCAGATTCTCAGGGTCCGGAAGCGGGACTGAA
TTTACACTAACGATCAGCAGCCTCCAGCCCGAGGACTTCGCCACCTATTATTG
CCAGCAGATTAACTCTTATCCTTTTACATTTGGCCAGGGTACTAAAGTAGAGA
TTAAGagaactgtggcggcgccatctgtcttcatcttcccgccatctgatgagcagttgaaatccggaactgcctctgttgtgt
gcctgctgaataacttctatcccagagaggccaaagtacagtggaaggtggataacgccctccaatcgggtaactcccaggaga
gtgtcacagagcaggacagcaaggacagcacctacagcctcagcagcaccctgacgctgagcaaagcagactacgagaaac
acaaagtctacgcctgcgaagtcacccatcagggcctgagctcgcccgtcacaaagagcttcaacaggggagagtgc
SEQ ID NO: 90
(DNA of HC for Ab4)
CAAGTGCAGCTGCAGGAGAGTGGACCAGGTCTCGTGAAGCCTAGTGAGACAC
TTTCACTCACTTGTACAGTAAGTGGCGGTTCTATCAGCGGGTATTACTGGTCC
TGGATTCGGCAGCCTCCCGGGAAGGGATTAGAATGGATCGGATATATCTATT
ATTCCGGCAGCACCAATTACAACCCCTCTCTAAAATCCAGGGTTACGATAAG
CGTGGATACCTCTAAAAACCAGTTTTCGTTGAAGCTGTCTAGCGTCACCGCAG
CTGACACCGCCGTGTACTACTGCGCTAGACTGGGCATCTTCTTTGACGCCTTC
GATATTTGGGGGCAGGGCACTCTGGTCACAGTTTCCTCAgcctccaccaagggcccatcg
gtcttccccctggcaccctcctccaagagcacctctgggggcacagcggccctgggctgcctggtcaaggactacttccccgaa
ccggtgacggtgtcgtggaactcaggcGCGctgaccagcggcgtgcacaccttcccggctgtcctacagtcctcaggactct
actccctcagcagcgtggtgaccgtgccctccagcagcttgggcacccagacctacatctgcaacgtgaatcacaagcccagc
aacaccaaggtggacaagagagttgagcccaaatcttgtgacaaaactcacacatgcccaccgtgcccagcacctgaagccgc
cgggggaccgtcagtcttcctcttccccccaaaacccaaggacaccctcatgatctcccggacccctgaggtcacatgcgtggt
ggtgtccgtgagccacgaagaccctgaggtcaagttcaactggtatgtggacggcgtggaggtgcataatgccaagacaaagc
cgcgggaggagcagtacaacagcacgtaccgtgtggtcagcgtcctcaccgtcctgcaccaagactggctgaatggcaagga
gtacaagtgcaaggtctccaacaaagccctcccagcccccatcgagaaaaccatctccaaagccaaagggcagccccgagaa
ccacaggtgtacaccctgcccccatcccgggaggagatgaccaagaaccaagtcagcctgacctgcctggtcaaaggcttctat
cccagcgacatcgccgtggagtgggagagcaatgggcagccggagaacaactacaagaccacgcctcccgtgctggactcc
gacggctccttcttcctctattccaagctcaccgtggacaagagcaggtggcagcaggggaacgtcttctcatgctccgtgatgc
atgaggctctgcacaaccactacacgcagaagagcctctccctgtctccgggcaaa
SEQ ID NO: 91
(DNA of LC for Ab4)
GATATACAACTAACACAGTCTCCTTCCTTCTTGTCTGCTAGTGTGGGCGACAG
GGTCACAATCACTTGCCGGGCAAGCCAGGGAATTTCCTCCTACCTGGCATGGT
ATCAGCAGAAGCCCGGGAAAGCTCCCAAGCTCCTGATCTATGCCGCCAGCAC
GCTTCAGAGCGGGGTGCCATCCAGATTTTCGGGTAGCGGCTCTGGCACTGAG
TTTACCCTCACAATTAGTTCACTGCAGCCTGAAGACTTTGCCACCTACTACTG
TCAACAGTTCAACTCATATCCATGGACCTTCGGTCAGGGAACCAAAGTTGAG
ATCAAGagaactgtggcggcgccatctgtcttcatcttcccgccatctgatgagcagttgaaatccggaactgcctctgttgt
gtgcctgctgaataacttctatcccagagaggccaaagtacagtggaaggtggataacgccctccaatcgggtaactcccagga
gagtgtcacagagcaggacagcaaggacagcacctacagcctcagcagcaccctgacgctgagcaaagcagactacgagaa
acacaaagtctacgcctgcgaagtcacccatcagggcctgagctcgcccgtcacaaagagcttcaacaggggagagtgc
SEQ ID NO: 92
(DNA of HC for Ab5)
CAGGTCCAGCTGGTTCAGTCCGGAGCAGAGGTAAAAAAACCAGGAGCTAGTG
TCAAGGTGTCCTGTAAGGCGTCTGGGTACACTTTTACTTCATACTATGTTCAC
TGGGTGCGACAGGCCCCTGGGCAGGGCCTCGAATGGATGGGCATCATAAACC
CCAGTATCATTTCCACCAGCTACGCTCAAAAGTTTCAAGCCCGGGTGACCATG
ACGAGGGATACCTCGACATCAACTGTCTATATGGAGCTATCCTCTTTGAGATC
TGAAGATACAGCCGTGTATTACTGCGCACGCGGCCTTAATTTCGACGCTTTCG
ACATTTGGGGTCAGGGTACACTGGTGACCGTGAGCAGCgcctccaccaagggcccatcgg
tcttccccctggcaccctcctccaagagcacctctgggggcacagcggccctgggctgcctggtcaaggactacttccccgaac
cggtgacggtgtcgtggaactcaggcGCGctgaccagcggcgtgcacaccttcccggctgtcctacagtcctcaggactcta
ctccctcagcagcgtggtgaccgtgccctccagcagcttgggcacccagacctacatctgcaacgtgaatcacaagcccagca
acaccaaggtggacaagagagttgagcccaaatcttgtgacaaaactcacacatgcccaccgtgcccagcacctgaagccgcc
gggggaccgtcagtcttcctcttccccccaaaacccaaggacaccctcatgatctcccggacccctgaggtcacatgcgtggtg
gtgtccgtgagccacgaagaccctgaggtcaagttcaactggtatgtggacggcgtggaggtgcataatgccaagacaaagcc
gcgggaggagcagtacaacagcacgtaccgtgtggtcagcgtcctcaccgtcctgcaccaagactggctgaatggcaaggag
tacaagtgcaaggtctccaacaaagccctcccagcccccatcgagaaaaccatctccaaagccaaagggcagccccgagaac
cacaggtgtacaccctgcccccatcccgggaggagatgaccaagaaccaagtcagcctgacctgcctggtcaaaggcttctatc
ccagcgacatcgccgtggagtgggagagcaatgggcagccggagaacaactacaagaccacgcctcccgtgctggactccg
acggctccttcttcctctattccaagctcaccgtggacaagagcaggtggcagcaggggaacgtcttctcatgctccgtgatgcat
gaggctctgcacaaccactacacgcagaagagcctctccctgtctccgggcaaa
SEQ ID NO: 93
(DNA of LC for Ab5)
GACATCCAGTTAACTCAGTCTCCTAGCTTCCTGAGCGCTTCTGTTGGAGATAG
AGTCACAATTACATGTAGGGCCTCCCAGGGGATTTCATCATATCTGGCTTGGT
ATCAACAGAAGCCTGGCAAAGCACCAAAGCTCCTGATCTATGCCGCATCTAC
CCTACAATCGGGTGTGCCCTCCCGGTTCAGTGGCTCCGGGAGTGGAACGGAA
TTTACCCTTACCATCTCCAGCTTGCAGCCCGAGGACTTCGCCACATACTACTG
CCAGCAGCTCAATAACTACCCATTTACTTTTGGTCAGGGCACCAAAGTGGAG
ATAAAGagaactgtggcggcgccatctgtcttcatcttcccgccatctgatgagcagttgaaatccggaactgcctctgttgt
gtgcctgctgaataacttctatcccagagaggccaaagtacagtggaaggtggataacgccctccaatcgggtaactcccagga
gagtgtcacagagcaggacagcaaggacagcacctacagcctcagcagcaccctgacgctgagcaaagcagactacgagaa
acacaaagtctacgcctgcgaagtcacccatcagggcctgagctcgcccgtcacaaagagcttcaacaggggagagtgc
SEQ ID NO: 94
(DNA of HC for Ab6)
CAAGTTCAGCTGGTGCAATCCGGAGCTGAGGTCAAGAAGCCTGGTGCAAGCG
TGAAAGTGAGTTGTAAAGCCTCAGGCTATACCTTCACTGGGTATTACATGCAC
TGGGTTCGGCAGGCTCCAGGACAGGGATTGGAATGGATGGGCATCATTAACC
CCAGCGGGGGTTCCACAACGTACGCCCAGAAGTTTCAGGGCCGTGTGACCAT
GACACGCGATACTTCTACATCTACCGTATACATGGAACTCAGTTCCCTGCGAT
CTGAGGACACTGCAGTCTATTACTGCGCCAGGGGCAGACTTGGCACCTATTTC
GACTACTGGGGTCAGGGGACCCTAGTCACAGTGAGCTCAgcctccaccaagggcccatcg
gtcttccccctggcaccctcctccaagagcacctctgggggcacagcggccctgggctgcctggtcaaggactacttccccgaa
ccggtgacggtgtcgtggaactcaggcGCGctgaccagcggcgtgcacaccttcccggctgtcctacagtcctcaggactct
actccctcagcagcgtggtgaccgtgccctccagcagcttgggcacccagacctacatctgcaacgtgaatcacaagcccagc
aacaccaaggtggacaagagagttgagcccaaatcttgtgacaaaactcacacatgcccaccgtgcccagcacctgaagccgc
cgggggaccgtcagtcttcctcttccccccaaaacccaaggacaccctcatgatctcccggacccctgaggtcacatgcgtggt
ggtgtccgtgagccacgaagaccctgaggtcaagttcaactggtatgtggacggcgtggaggtgcataatgccaagacaaagc
cgcgggaggagcagtacaacagcacgtaccgtgtggtcagcgtcctcaccgtcctgcaccaagactggctgaatggcaagga
gtacaagtgcaaggtctccaacaaagccctcccagcccccatcgagaaaaccatctccaaagccaaagggcagccccgagaa
ccacaggtgtacaccctgcccccatcccgggaggagatgaccaagaaccaagtcagcctgacctgcctggtcaaaggcttctat
cccagcgacatcgccgtggagtgggagagcaatgggcagccggagaacaactacaagaccacgcctcccgtgctggactcc
gacggctccttcttcctctattccaagctcaccgtggacaagagcaggtggcagcaggggaacgtcttctcatgctccgtgatgc
atgaggctctgcacaaccactacacgcagaagagcctctccctgtctccgggcaaa
SEQ ID NO: 95
(DNA of LC for Ab6)
AGCTACGTCCTTACTCAGCCACCTAGTGTGTCAGTAGCGCCTGGGCAAACCGC
TCGCATAACTTGCGGGGGAAACAACATCGGCTCTAAATCCGTCCACTGGTAC
CAGCAGAAGCCCGGCCAGGCACCCGTATTGGTTGTTTACGACGATAGCGACA
GACCGTCCGGTATCCCAGAGCGGTTTTCCGGTAGTAATTCTGGAAATACAGCT
ACCCTGACAATTTCTAGGGTGGAAGCCGGGGATGAGGCCGACTATTATTGTC
AAGTGTGGGATCGATCAAGCGACCAGGTGGTCTTCGGCGGAGGCACGAAGCT
GACCGTGCTCggccagcccaaggctgccccctcggtcactctgttcccgccctcctctgaggagcttcaagccaacaa
ggccacactggtgtgtctcataagtgacttctacccgggagccgtgacagtggcctggaaggcagatagcagccccgtcaagg
cgggagtggagacaaccacaccctccaaacaaagcaacaacaagtacgcggccagcagctatctgagcctgacgcctgagc
agtggaagtcccacagaagctacagctgccaggtcacgcatgaagggagcaccgtggagaagacagtggcccctacagaat
gttca
SEQ ID NO: 96
(DNA of HC for Ab7)
CAGGTGCAACTGGTGCAGAGTGGAGCCGAGGTGAAGAAGCCCGGCGCTTCG
GTCAAGGTTTCATGCAAAGCATCCGGGTATACCTTCACTAACTACTATATGCA
CTGGGTTCGCCAGGCCCCAGGTCAAGGCTTGGAATGGATGGGAATCATAAAT
CCTTCTAGCGGTTCTGCATCCTACGCTCAGAAATTTCAGGGACGTGTCACCAT
GACAAGAGATACATCTACTTCAACCGTCTACATGGAGCTAAGTTCCCTGAGG
TCCGAAGACACCGCCGTATATTACTGTGCGCGGGAGGGCGTGGGCCGAGACA
TTCTCCAGGCTTTCGATATCTGGGGGCAGGGGACGCTTGTGACAGTGAGCAG
Cgcctccaccaagggcccatcggtcttccccctggcaccctcctccaagagcacctctgggggcacagcggccctgggctgc
ctggtcaaggactacttccccgaaccggtgacggtgtcgtggaactcaggcGCGctgaccagcggcgtgcacaccttcccg
gctgtcctacagtcctcaggactctactccctcagcagcgtggtgaccgtgccctccagcagcttgggcacccagacctacatct
gcaacgtgaatcacaagcccagcaacaccaaggtggacaagagagttgagcccaaatcttgtgacaaaactcacacatgccca
ccgtgcccagcacctgaagccgccgggggaccgtcagtcttcctcttccccccaaaacccaaggacaccctcatgatctcccg
gacccctgaggtcacatgcgtggtggtgtccgtgagccacgaagaccctgaggtcaagttcaactggtatgtggacggcgtgg
aggtgcataatgccaagacaaagccgcgggaggagcagtacaacagcacgtaccgtgtggtcagcgtcctcaccgtcctgca
ccaagactggctgaatggcaaggagtacaagtgcaaggtctccaacaaagccctcccagcccccatcgagaaaaccatctcca
aagccaaagggcagccccgagaaccacaggtgtacaccctgcccccatcccgggaggagatgaccaagaaccaagtcagc
ctgacctgcctggtcaaaggcttctatcccagcgacatcgccgtggagtgggagagcaatgggcagccggagaacaactacaa
gaccacgcctcccgtgctggactccgacggctccttcttcctctattccaagctcaccgtggacaagagcaggtggcagcaggg
gaacgtcttctcatgctccgtgatgcatgaggctctgcacaaccactacacgcagaagagcctctccctgtctccgggcaaa
SEQ ID NO: 97
(DNA of LC for Ab7)
CAGTCTGTTCTCACACAACCACCCTCAGTAAGCGAGGCTCCCCGGCAGAGAG
TCACAATCTCCTGTAGCGGAAGTAGTTCAAACATAGGGAATAATGCGGTGAA
CTGGTATCAGCAGTTGCCCGGTAAAGCCCCTAAACTGCTCATTTACTACGACG
ACTTACTACCTTCCGGCGTGAGCGACAGGTTTTCAGGAAGCAAGTCTGGCACT
TCGGCCTCCCTGGCAATCTCTGGACTTCAGAGTGAGGATGAAGCTGACTATTA
CTGCGCAGCCTGGGATGATTCCTTGAACGGCCACGTCTTCGGTGGCGGGACC
AAGTTGACCGTGCTGggccagcccaaggctgccccctcggtcactctgttcccgccctcctctgaggagcttcaa
gccaacaaggccacactggtgtgtctcataagtgacttctacccgggagccgtgacagtggcctggaaggcagatagcagccc
cgtcaaggcgggagtggagacaaccacaccctccaaacaaagcaacaacaagtacgcggccagcagctatctgagcctgac
gcctgagcagtggaagtcccacagaagctacagctgccaggtcacgcatgaagggagcaccgtggagaagacagtggcccc
tacagaatgttca
SEQ ID NO: 98
(DNA of HC for Ab8)
CAGGTGCAGCTCGTCCAGAGTGGGGCCGAGGTGAAAAAGCCTGGCGCTTCGG
TCAAGGTTTCATGCAAAGCCTCCGGTTACACCTTCACATCCTATTATATCCAC
TGGGTGAGACAAGCTCCAGGACAGGGCCTGGAATGGATGGGAATTATAAACC
CCATTAGCGGTCGAACCTCTTCTGCCCAGAAGTTCCAGGGACGTGTGACTATG
ACTCGGGATACATCCACAAGCACCGTATACATGGAGCTATCTTCACTTAGGA
GTGAGGACACTGCAGTTTACTATTGTGCGAAGGAAGGTGTCGGGGGCGAGCT
GTTGCGCGCATTTGACATCTGGGGGCAAGGCACCCTGGTGACGGTGTCCAGCg
cctccaccaagggcccatcggtcttccccctggcaccctcctccaagagcacctctgggggcacagcggccctgggctgcctg
gtcaaggactacttccccgaaccggtgacggtgtcgtggaactcaggcGCGctgaccagcggcgtgcacaccttcccggct
gtcctacagtcctcaggactctactccctcagcagcgtggtgaccgtgccctccagcagcttgggcacccagacctacatctgca
acgtgaatcacaagcccagcaacaccaaggtggacaagagagttgagcccaaatcttgtgacaaaactcacacatgcccaccg
tgcccagcacctgaagccgccgggggaccgtcagtcttcctcttccccccaaaacccaaggacaccctcatgatctcccggacc
cctgaggtcacatgcgtggtggtgtccgtgagccacgaagaccctgaggtcaagttcaactggtatgtggacggcgtggaggtg
cataatgccaagacaaagccgcgggaggagcagtacaacagcacgtaccgtgtggtcagcgtcctcaccgtcctgcaccaag
actggctgaatggcaaggagtacaagtgcaaggtctccaacaaagccctcccagcccccatcgagaaaaccatctccaaagcc
aaagggcagccccgagaaccacaggtgtacaccctgcccccatcccgggaggagatgaccaagaaccaagtcagcctgacc
tgcctggtcaaaggcttctatcccagcgacatcgccgtggagtgggagagcaatgggcagccggagaacaactacaagacca
cgcctcccgtgctggactccgacggctccttcttcctctattccaagctcaccgtggacaagagcaggtggcagcaggggaacg
tcttctcatgctccgtgatgcatgaggctctgcacaaccactacacgcagaagagcctctccctgtctccgggcaaa
SEQ ID NO: 99
(DNA of LC for Ab8)
CAGAGTGTGCTGACCCAGCCCCCAAGCGTCTCCGAGGCACCCAGACAGAGGG
TGACAATCTCCTGCTCCGGAAGCAGTTCAAACATAGGCAACAACGCCGTAAA
TTGGTACCAGCAGCTGCCAGGTAAGGCCCCTAAACTTCTGATCTATTACGATG
ATTTGCTCCCTTCAGGTGTCTCTGACCGCTTTTCCGGAAGCAAATCTGGGACC
AGCGCTTCTTTGGCAATTTCTGGCCTGCAATCGGAAGACGAGGCTGATTACTA
TTGTGCCGCTTGGGACGACAGTTTAAATGGGTTCGTTTTCGGCGGCGGAACAA
AGCTAACTGTGCTCggccagcccaaggctgccccctcggtcactctgttcccgccctcctctgaggagcttcaagc
caacaaggccacactggtgtgtctcataagtgacttctacccgggagccgtgacagtggcctggaaggcagatagcagccccg
tcaaggcgggagtggagacaaccacaccctccaaacaaagcaacaacaagtacgcggccagcagctatctgagcctgacgc
ctgagcagtggaagtcccacagaagctacagctgccaggtcacgcatgaagggagcaccgtggagaagacagtggccccta
cagaatgttca
SEQ ID NO: 100:
-Ala-Leu-Ala-Leu- (also known as ALAL in one letter code)
SEQ ID NO: 101:
-Leu-Ala-Leu-Ala- (also known as LALA in one letter code)
SEQ ID NO: 102
-Gly-Gly-Phe-Gly- (also known as GGFG in one letter code)
SEQ ID NO: 103
-Gly-Phe-Leu-Gly- (also known as GFLG in one letter code)
SEQ ID NO: 104
-Gly-Leu-Phe-Gly- (also known as GLFG in one letter code)
SEQ ID NO: 105
-Ala-Ala-Ala-Ala- (also known as AAAA in one letter code)
SEQ ID NO: 106
-Gly-Ala-Gly-Gly- (also known as GAGG in one letter code)
SEQ ID NO: 107
-Gly-Gly-Ala-Gly- (also known as GGAG in one letter code)
SEQ ID NO: 108
-Gly-Val-Gly-Gly- (also known as GVGG in one letter code)
SEQ ID NO: 109
-Gly-Gly-Val-Gly- (also known as GGVG in one letter code)
SEQ ID NO: 110:
-Gly-Phe-Gly-Gly- (also known as GFGG in one letter code)

We claim:

1. An antibody that binds human nectin-4, wherein the antibody comprises a heavy chain variable region (HCVR) and a light chain variable region (LCVR), wherein the HCVR comprises heavy chain complementarity determining regions (HCDR) HCDR1, HCDR2, and HCDR3, and the LCVR comprises light chain complementarity determining regions (LCDR) LCDR1, LCDR2, and LCDR3, wherein:
a) the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9;
b) the HCDR1 comprises SEQ ID NO: 18, the HCDR2 comprises SEQ ID NO: 19, the HCDR3 comprises SEQ ID NO: 20, the LCDR1 comprises SEQ ID NO: 21, the LCDR2 comprises SEQ ID NO: 22, and the LCDR3 comprises SEQ ID NO: 23;
c) the HCDR1 comprises SEQ ID NO: 28, the HCDR2 comprises SEQ ID NO: 29, the HCDR3 comprises SEQ ID NO: 30, the LCDR1 comprises SEQ ID NO: 31, the LCDR2 comprises SEQ ID NO: 32, and the LCDR3 comprises SEQ ID NO: 33;
d) the HCDR1 comprises SEQ ID NO: 38, the HCDR2 comprises SEQ ID NO: 39, the HCDR3 comprises SEQ ID NO: 40, the LCDR1 comprises SEQ ID NO: 41, the LCDR2 comprises SEQ ID NO: 42, and the LCDR3 comprises SEQ ID NO: 43;
e) the HCDR1 comprises SEQ ID NO: 48, the HCDR2 comprises SEQ ID NO: 49, the HCDR3 comprises SEQ ID NO: 50, the LCDR1 comprises SEQ ID NO: 41, the LCDR2 comprises SEQ ID NO: 42, and the LCDR3 comprises SEQ ID NO: 51;
f) the HCDR1 comprises SEQ ID NO: 56, the HCDR2 comprises SEQ ID NO: 57, the HCDR3 comprises SEQ ID NO: 58, the LCDR1 comprises SEQ ID NO: 59, the LCDR2 comprises SEQ ID NO: 60, and the LCDR3 comprises SEQ ID NO: 61;
g) the HCDR1 comprises SEQ ID NO: 66, the HCDR2 comprises SEQ ID NO: 67, the HCDR3 comprises SEQ ID NO: 68, the LCDR1 comprises SEQ ID NO: 69, the LCDR2 comprises SEQ ID NO: 70, and the LCDR3 comprises SEQ ID NO: 71; or
h) the HCDR1 comprises SEQ ID NO: 76, the HCDR2 comprises SEQ ID NO: 77, the HCDR3 comprises SEQ ID NO: 78, the LCDR1 comprises SEQ ID NO: 69, the LCDR2 comprises SEQ ID NO: 70, and the LCDR3 comprises SEQ ID NO: 79.

2. The antibody of claim 1, wherein the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9.

3. The antibody of claim 1, wherein the HCDR1 comprises SEQ ID NO: 18, the HCDR2 comprises SEQ ID NO: 19, the HCDR3 comprises SEQ ID NO: 20, the LCDR1 comprises SEQ ID NO: 21, the LCDR2 comprises SEQ ID NO: 22, and the LCDR3 comprises SEQ ID NO: 23.

4. The antibody of claim 1, wherein the HCDR1 comprises SEQ ID NO: 28, the HCDR2 comprises SEQ ID NO: 29, the HCDR3 comprises SEQ ID NO: 30, the LCDR1 comprises SEQ ID NO: 31, the LCDR2 comprises SEQ ID NO: 32, and the LCDR3 comprises SEQ ID NO: 33.

5. The antibody of claim 1, wherein the HCDR1 comprises SEQ ID NO: 38, the HCDR2 comprises SEQ ID NO: 39, the HCDR3 comprises SEQ ID NO: 40, the LCDR1 comprises SEQ ID NO: 41, the LCDR2 comprises SEQ ID NO: 42, and the LCDR3 comprises SEQ ID NO: 43.

6. The antibody of claim 1, wherein the HCDR1 comprises SEQ ID NO: 48, the HCDR2 comprises SEQ ID NO: 49, the HCDR3 comprises SEQ ID NO: 50, the LCDR1 comprises SEQ ID NO: 41, the LCDR2 comprises SEQ ID NO: 42, and the LCDR3 comprises SEQ ID NO: 51.

7. The antibody of claim 1, wherein the HCDR1 comprises SEQ ID NO: 56, the HCDR2 comprises SEQ ID NO: 57, the HCDR3 comprises SEQ ID NO: 58, the LCDR1 comprises SEQ ID NO: 59, the LCDR2 comprises SEQ ID NO: 60, and the LCDR3 comprises SEQ ID NO: 61.

8. The antibody of claim 1, wherein the HCDR1 comprises SEQ ID NO: 66, the HCDR2 comprises SEQ ID NO: 67, the HCDR3 comprises SEQ ID NO: 68, the LCDR1 comprises SEQ ID NO: 69, the LCDR2 comprises SEQ ID NO: 70, and the LCDR3 comprises SEQ ID NO: 71.

9. The antibody of claim 1, wherein the HCDR1 comprises SEQ ID NO: 76, the HCDR2 comprises SEQ ID NO: 77, the HCDR3 comprises SEQ ID NO: 78, the LCDR1 comprises SEQ ID NO: 69, the LCDR2 comprises SEQ ID NO: 70, and the LCDR3 comprises SEQ ID NO: 79.

10. The antibody of claim 1, wherein:
a) the HCVR comprises SEQ ID NO: 10 and the LCVR comprises SEQ ID NO: 11;
b) the HCVR comprises SEQ ID NO: 14 and the LCVR comprises SEQ ID NO: 15;
c) the HCVR comprises SEQ ID NO: 24 and the LCVR comprises SEQ ID NO: 25;
d) the HCVR comprises SEQ ID NO: 34 and the LCVR comprises SEQ ID NO: 35;
e) the HCVR comprises SEQ ID NO: 44 and the LCVR comprises SEQ ID NO: 45;
f) the HCVR comprises SEQ ID NO: 52 and the LCVR comprises SEQ ID NO: 53;
g) the HCVR comprises SEQ ID NO: 62 and the LCVR comprises SEQ ID NO: 63;
h) the HCVR comprises SEQ ID NO: 72 and the LCVR comprises SEQ ID NO: 73; or
i) the HCVR comprises SEQ ID NO: 80 and the LCVR comprises SEQ ID NO: 81.

11. The antibody of claim 10, wherein the antibody comprises a HCVR comprising SEQ ID NO: 10 and a LCVR comprising SEQ ID NO: 11.

12. The antibody of claim 10, wherein the antibody comprises a HCVR comprising SEQ ID NO: 14 and a LCVR comprising SEQ ID NO: 15.

13. The antibody of claim 10, wherein the antibody comprises a HCVR comprising SEQ ID NO: 24 and a LCVR comprising SEQ ID NO: 25.

14. The antibody of claim 10, wherein the antibody comprises a HCVR comprising SEQ ID NO: 34 and a LCVR comprising SEQ ID NO: 35.

15. The antibody of claim 10, wherein the antibody comprises a HCVR comprising SEQ ID NO: 44 and a LCVR comprising SEQ ID NO: 45.

16. The antibody of claim 10, wherein the antibody comprises a HCVR comprising SEQ ID NO: 52 and a LCVR comprising SEQ ID NO: 53.

17. The antibody of claim 10, wherein the antibody comprises a HCVR comprising SEQ ID NO: 62 and a LCVR comprising SEQ ID NO: 63.

18. The antibody of claim 10, wherein the antibody comprises a HCVR comprising SEQ ID NO: 72 and a LCVR comprising SEQ ID NO: 73.

19. The antibody of claim 10, wherein the antibody comprises a HCVR comprising SEQ ID NO: 80 and a LCVR comprising SEQ ID NO: 81.

20. The antibody of claim 10, wherein the antibody has a human IgG1 or IgG4 isotype.

21. The antibody of claim 20, wherein the antibody has a human IgG1 isotype.

22. The antibody of claim 21, comprising alanine at residues 234 and 235 (according to EU Index numbering).

23. The antibody of claim 22, further comprising serine at position 265 (according to EU Index numbering).

24. The antibody of claim 1, wherein the antibody comprises a heavy chain (HC) and a light chain (LC), wherein:
a) the HC comprises amino acids 2-444 of SEQ ID NO: 2 and the LC comprises amino acids 2-215 of SEQ ID NO: 3;
b) the HC comprises amino acids 2-444 of SEQ ID NO: 12 and the LC comprises amino acids 2-215 of SEQ ID NO: 13;
c) the HC comprises amino acids 2-443 of SEQ ID NO: 16 and the LC consists of SEQ ID NO: 17;
d) the HC comprises amino acids 2-447 of SEQ ID NO: 26 and the LC consists of SEQ ID NO: 27;
e) the HC comprises amino acids 2-446 of SEQ ID NO: 36 and the LC consists of SEQ ID NO: 37;
f) the HC comprises amino acids 2-446 of SEQ ID NO: 46 and the LC consists of SEQ ID NO: 47;
g) the HC comprises amino acids 2-446 of SEQ ID NO: 54 and the LC consists of SEQ ID NO: 55;
h) the HC comprises amino acids 2-450 of SEQ ID NO: 64 and the LC comprises amino acids 2-216 of SEQ ID NO: 65; or
i) the HC comprises amino acids 2-450 of SEQ ID NO: 74 and the LC comprises amino acids 2-216 of SEQ ID NO: 75.

25. The antibody of claim 24, wherein:
a) the HC consists of SEQ ID NO: 2 and the LC consists of SEQ ID NO: 3;
b) the HC consists of SEQ ID NO: 12 and the LC consists of SEQ ID NO: 13;
c) the HC consists of SEQ ID NO: 16 and the LC consists of SEQ ID NO: 17;
d) the HC consists of SEQ ID NO: 26 and the LC consists of SEQ ID NO: 27;
e) the HC consists of SEQ ID NO: 36 and the LC consists of SEQ ID NO: 37;
f) the HC consists of SEQ ID NO: 46 and the LC consists of SEQ ID NO: 47;
g) the HC consists of SEQ ID NO: 54 and the LC consists of SEQ ID NO: 55;
h) the HC consists of SEQ ID NO: 64 and the LC consists of SEQ ID NO: 65; or
i) the HC consists of SEQ ID NO: 74 and the LC consists of SEQ ID NO: 75.

26. The antibody of claim 25, wherein the HC consists of SEQ ID NO: 2 and the LC consists of SEQ ID NO: 3.

27. The antibody of claim 25, wherein the HC consists of SEQ ID NO: 12 and the LC consists of SEQ ID NO: 13.

28. The antibody of claim 25, wherein the HC consists of SEQ ID NO: 16 and the LC consists of SEQ ID NO: 17.

29. The antibody of claim 25, wherein the HC consists of SEQ ID NO: 26 and the LC consists of SEQ ID NO: 27.

30. The antibody of claim 25, wherein the HC consists of SEQ ID NO: 36 and the LC consists of SEQ ID NO: 37.

31. The antibody of claim 25, wherein the HC consists of SEQ ID NO: 46 and the LC consists of SEQ ID NO: 47.

32. The antibody of claim 25, wherein the HC consists of SEQ ID NO: 54 and the LC consists of SEQ ID NO: 55.

33. The antibody of claim 25, wherein the HC consists of SEQ ID NO: 64 and the LC consists of SEQ ID NO: 65.

34. The antibody of claim 25, wherein the HC consists of SEQ ID NO: 74 and the LC consists of SEQ ID NO: 75.

35. An antibody-drug conjugate (ADC) comprising the antibody of claim 1 conjugated to a cytotoxic agent.

36. The ADC of claim 35, wherein the cytotoxic agent is selected from the group consisting of a microtubule inhibitor, a topoisomerase I inhibitor, a DNA damaging agent, a DNA alkylating agent, and a DNA minor groove binder.

37. The ADC of claim 36, wherein the cytotoxic agent is a topoisomerase I inhibitor.

38. The ADC of claim 37, wherein the topoisomerase I inhibitor is a camptothecin analog.

39. The ADC of claim 38, wherein the camptothecin analog comprises the Formula X-Y,
wherein:
Y is of the Formula 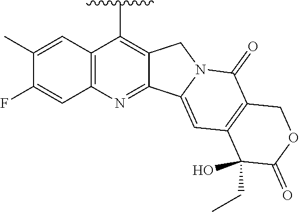 [SMILES: *c1c2c(nc3cc(F)c(C)cc13)-c1cc3c(c(=O)n1C2)COC(=O)[C@]3(O)CC]
and X is *—CH₂O—, *—(CH₂)₂O—, *—(CH₂)₃O—, *—(CH₂)₄O—, *—CH₂NH—, *—(CH₂)₂NH—, *—(CH₂)₃NH—, *—(CH₂)₄NH—, *—CH₂N(CH₃)—, *—(CH₂)₂N(CH₃)—, *—(CH₂)₃N(CH₃)—, *—(CH₂)₄N(CH₃)—, *—CH₂N(R¹)—, *—(CH₂)₂N(R¹)—, *—(CH₂)₃N(R¹)—, *—(CH₂)₄N(R¹)—,*—CH₂N(CH₃)C(═O)CH₂O—, *—CH₂N(R¹)C(═O)CH₂O—, *—CH₂NHC(═O)CH₂O—, *—CH₂NHC(═O)(CH₂)₂O—, *—CH₂NHC(═O)(CH₂)₃O—, *—CH₂NHC(═O)(CH₂)₄O—, *—CH₂NHC(═O)(CH₂)₅O—, *—CH₂NHC(═O)CH₂—, *—CH₂NHC(═O)(CH₂)₂—, *—CH₂NHC(═O)(CH₂)₃—, *—CH₂NHC(═)(CH₂)₄—, *—CH₂NHC(═O)(CH₂)₅—, *—CH₂SCH₂—, *—CH₂S(CH₂)₂—, *—CH₂S(CH₂)₃—, *—CH₂S(CH₂)₄—, or *—CH₂S(CH₂)₅—;
wherein * is the site covalently attached to Y and R¹ is benzyl.

40. The ADC of claim 39, wherein the camptothecin analog comprises any one of the following Formula:

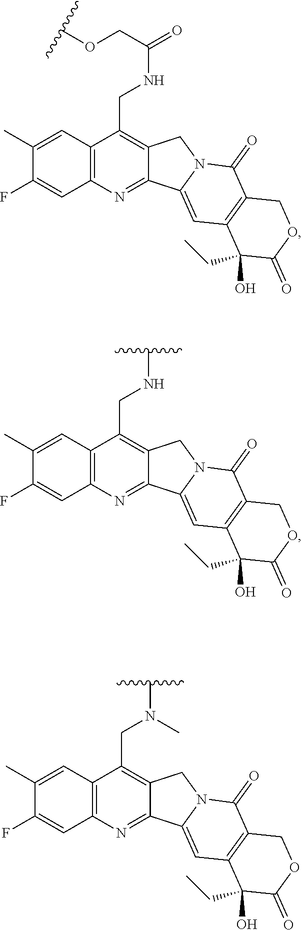 [SMILES: *OCC(=O)NCc1c2c(nc3cc(F)c(C)cc13)-c1cc3c(c(=O)n1C2)COC(=O)[C@]3(O)CC ; *NCc1c2c(nc3cc(F)c(C)cc13)-c1cc3c(c(=O)n1C2)COC(=O)[C@]3(O)CC ; *N(C)Cc1c2c(nc3cc(F)c(C)cc13)-c1cc3c(c(=O)n1C2)COC(=O)[C@]3(O)CC]

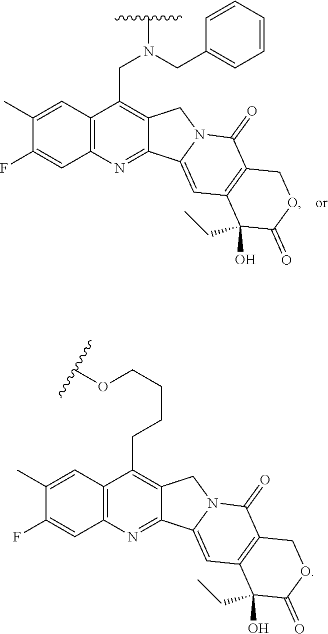 [SMILES: *N(Cc1ccccc1)Cc1c2c(nc3cc(F)c(C)cc13)-c1cc3c(c(=O)n1C2)COC(=O)[C@]3(O)CC ; *OCCCCc1c2c(nc3cc(F)c(C)cc13)-c1cc3c(c(=O)n1C2)COC(=O)[C@]3(O)CC]

41. The ADC of claim 40, wherein the camptothecin analog comprises the Formula:

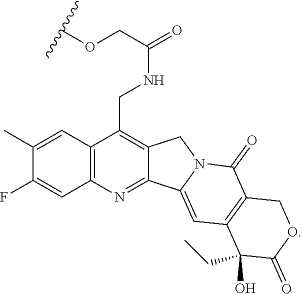 [SMILES: *OCC(=O)NCc1c2c(nc3cc(F)c(C)cc13)-c1cc3c(c(=O)n1C2)COC(=O)[C@]3(O)CC]

42. The ADC of claim 40, wherein the camptothecin analog comprises the Formula:

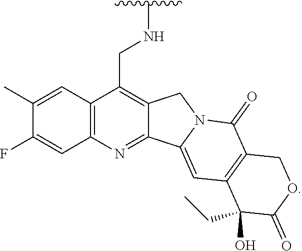 [SMILES: *NCc1c2c(nc3cc(F)c(C)cc13)-c1cc3c(c(=O)n1C2)COC(=O)[C@]3(O)CC]

43. The ADC of claim 40, wherein the camptothecin analog comprises the Formula:

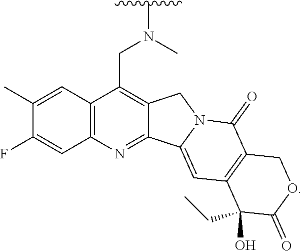 [SMILES: *N(C)Cc1c2c(nc3cc(F)c(C)cc13)-c1cc3c(c(=O)n1C2)COC(=O)[C@]3(O)CC]

44. The ADC of claim 40, wherein the camptothecin analog comprises the Formula:

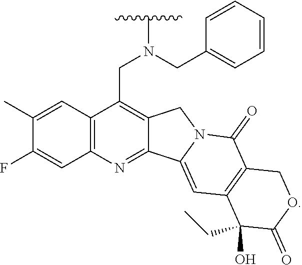 [SMILES: *N(Cc1ccccc1)Cc1c2c(nc3cc(F)c(C)cc13)-c1cc3c(c(=O)n1C2)COC(=O)[C@]3(O)CC]

45. The ADC of claim 40, wherein the camptothecin analog comprises the Formula:

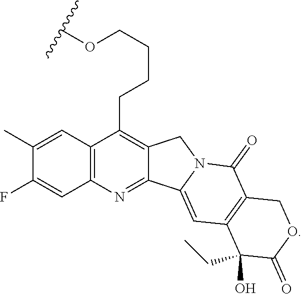 [SMILES: *OCCCCc1c2c(nc3cc(F)c(C)cc13)-c1cc3c(c(=O)n1C2)COC(=O)[C@]3(O)CC]

46. The ADC of claim 40, wherein the ADC further comprises a self-immolative spacer.

47. The ADC of claim 46, wherein the self-immolative spacer is —NH—CH₂— or absent.

48. The ADC of claim 40, wherein the ADC further comprises a linker which connects the antibody to the cytotoxic agent.

49. The ADC of claim 48, wherein the linker comprises a peptide unit.

50. The ADC of claim 49, wherein the peptide unit comprises Ala-Ala-Ala, Val-Cit, or Gly-Gly-Phe-Gly (SEQ ID NO: 102).

51. The ADC of claim 50, wherein the peptide unit comprises Ala-Ala-Ala.

52. The ADC of claim 50, wherein the peptide unit comprises Val-Cit.

53. The ADC of claim 50, wherein the peptide unit comprises Gly-Gly-Phe-Gly (SEQ ID NO: 102).

54. The ADC of claim 48, wherein the linker further comprises a spacer unit A between the antibody and the peptide unit.

55. The ADC of claim 54, wherein spacer unit A is of the Formula: 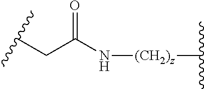
wherein z is from 1 to 5.

56. The ADC of claim 54, wherein spacer unit A is of the Formula: 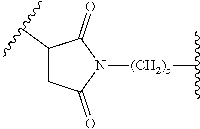
wherein z is from 1 to 5.

57. An antibody-drug conjugate (ADC), wherein the ADC is one of the Formula: 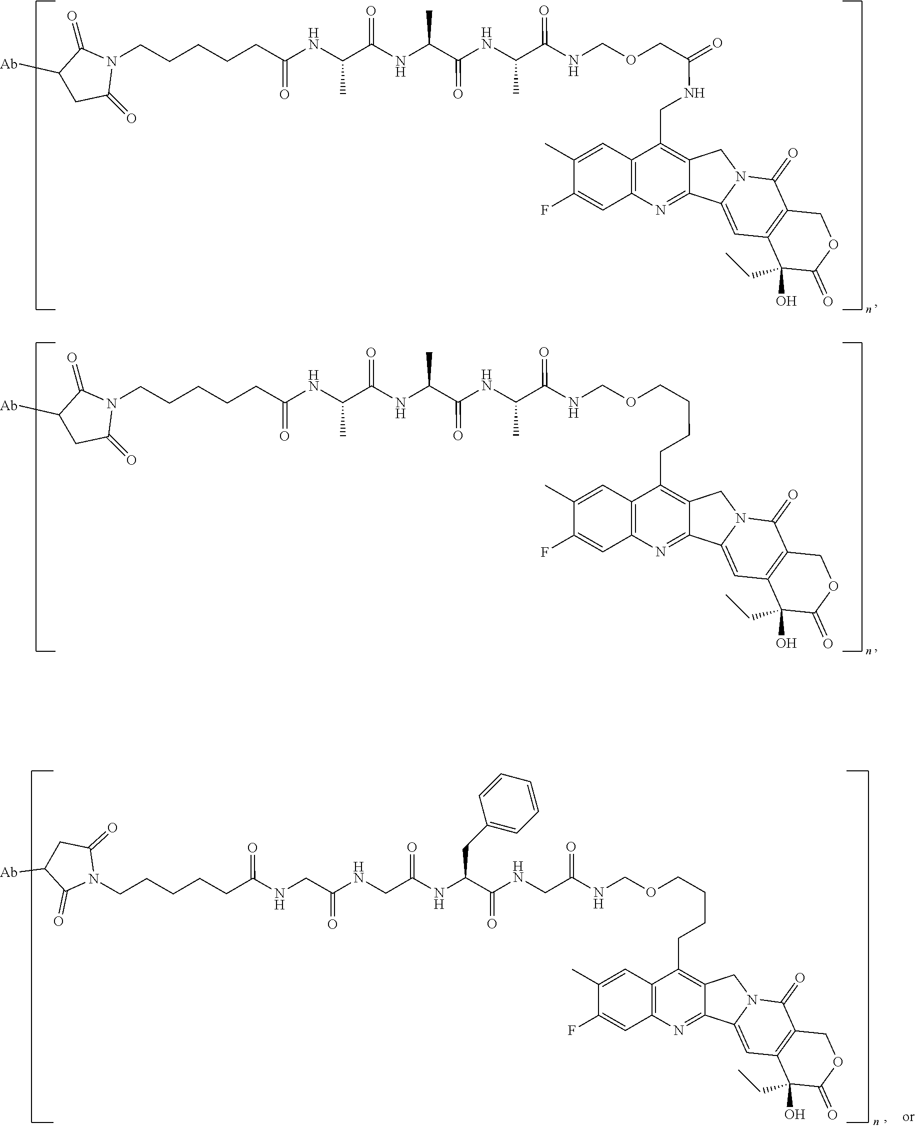 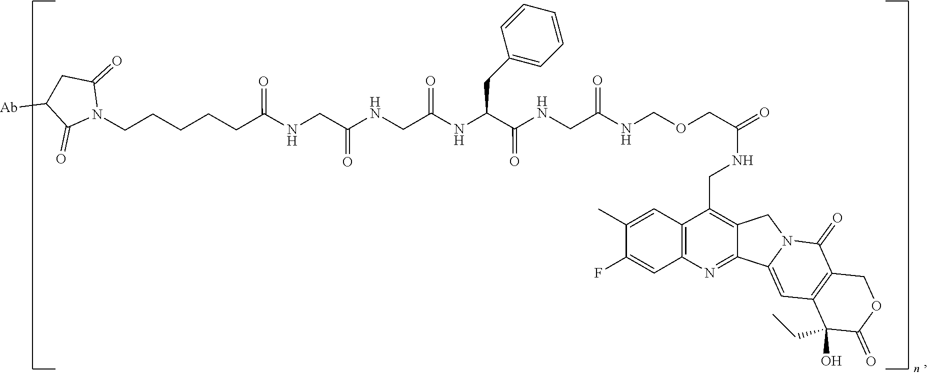
wherein:
Ab is the antibody of claim 1, and
n is from about 1 to 16.

58. The ADC of claim 57, wherein n is from about 2 to 12.

59. The ADC of claim 57, wherein n is from about 2 to 8.

60. The ADC of claim 59, wherein the ADC is of the Formula:

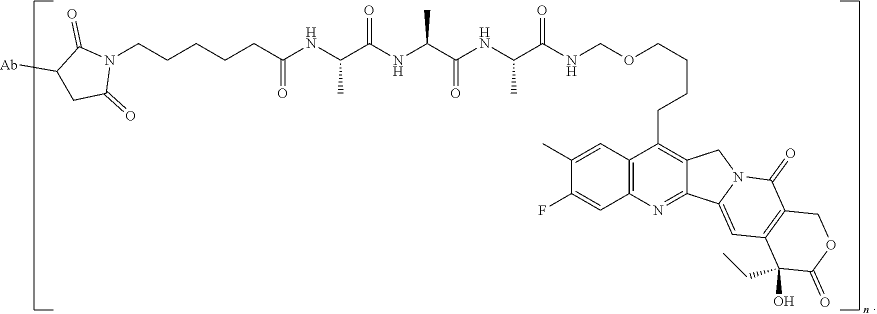

61. The ADC of claim 59, wherein the ADC is of the Formula:

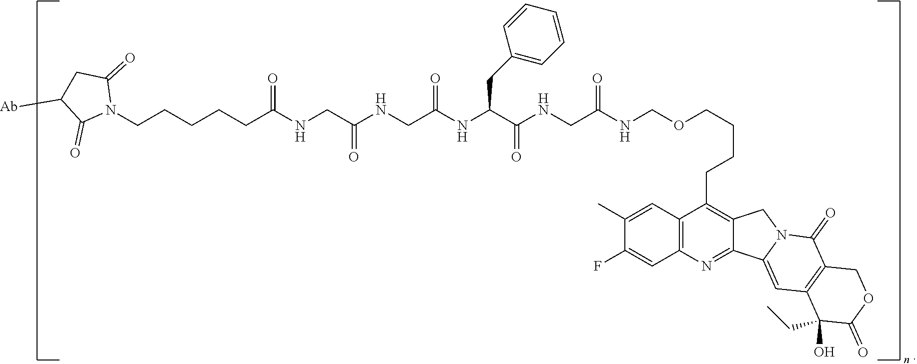

62. The ADC of claim 59, wherein the ADC is of the Formula:

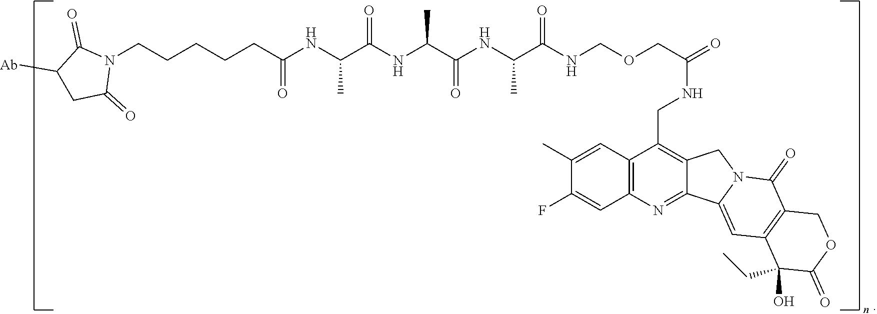

63. The ADC of claim 59, wherein the ADC is of the Formula:

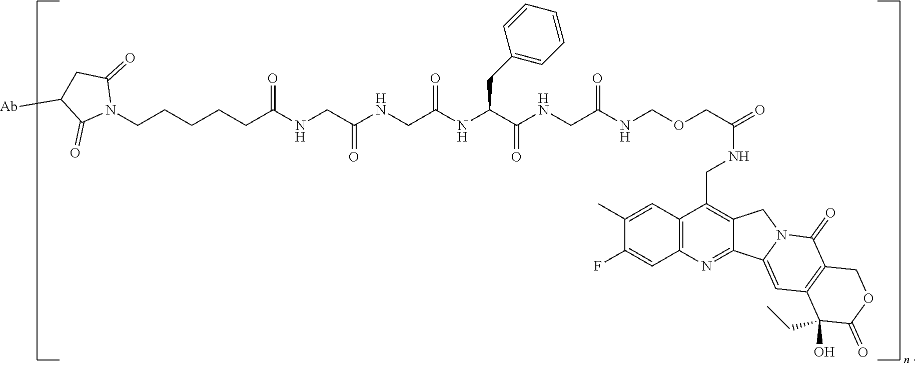

64. The ADC of claim 57, wherein n is about 2.

65. The ADC of claim 57, wherein n is about 4.

66. The ADC of claim 57, wherein n is about 6.

67. The ADC of claim 57, wherein n is about 8.

68. The ADC of claim 57, wherein connection to the antibody occurs through a thiol group on one or more cysteines of the antibody.

69. The ADC of claim 68, wherein the one or more cysteines are each a natural cysteine in the hinge region of the antibody.

70. The ADC of claim 69, wherein the Ab comprises a HC comprising amino acids 2-444 of SEQ ID NO: 2 and a LC comprising amino acids 2-215 of SEQ ID NO: 3, and wherein n is about 8.

71. The ADC of claim 70, wherein the Ab comprises a HC consisting of SEQ ID NO: 2 and a LC consisting of SEQ ID NO: 3.

72. The ADC of claim 69, wherein the Ab comprises a HC comprising amino acids 2-444 of SEQ ID NO: 12 and a LC comprising amino acids 2-215 of SEQ ID NO: 13, and wherein n is about 8.

73. The ADC of claim 72, wherein the Ab comprises a HC consisting of SEQ ID NO: 12 and a LC consisting of SEQ ID NO: 13.

74. The ADC of claim 69, wherein the Ab comprises a HC comprising amino acids 2-443 of SEQ ID NO: 16 and a LC consisting of SEQ ID NO: 17, and wherein n is about 8.

75. The ADC of claim 74, wherein the Ab comprises a HC consisting of SEQ ID NO: 16 and a LC consisting of SEQ ID NO: 17.

76. The ADC of claim 69, wherein the Ab comprising amino acids 2-447 of SEQ ID NO: 26 and a LC consisting of SEQ ID NO: 27, and wherein n is about 8.

77. The ADC of claim 76, wherein the Ab comprises a HC consisting of SEQ ID NO: 26 and a LC consisting of SEQ ID NO: 27.

78. The ADC of claim 69, wherein the Ab comprising amino acids 2-446 of SEQ ID NO: 36 and a LC consisting of SEQ ID NO: 37, and wherein n is about 8.

79. The ADC of claim 78, wherein the Ab comprises a HC consisting of SEQ ID NO: 36 and a LC consisting of SEQ ID NO: 37.

80. The ADC of claim 69, wherein the Ab comprising amino acids 2-446 of SEQ ID NO: 46 and a LC consisting of SEQ ID NO: 47, and wherein n is about 8.

81. The ADC of claim 80, wherein the Ab comprises a HC consisting of SEQ ID NO: 46 and a LC consisting of SEQ ID NO: 47.

82. The ADC of claim 69, wherein the Ab comprising amino acids 2-446 of SEQ ID NO: 54 and a LC consisting of SEQ ID NO: 55, and wherein n is about 8.

83. The ADC of claim 82, wherein the Ab comprises a HC consisting of SEQ ID NO: 54 and a LC consisting of SEQ ID NO: 55.

84. The ADC of claim 69, wherein the Ab comprising amino acids 2-450 of SEQ ID NO: 64 and a LC comprising amino acids 2-216 of SEQ ID NO: 65, and wherein n is about 8.

85. The ADC of claim 84, wherein the Ab comprises a HC consisting of SEQ ID NO: 64 and a LC consisting of SEQ ID NO: 65.

86. The ADC of claim 69, wherein the Ab comprising amino acids 2-450 of SEQ ID NO: 74 and a LC comprising amino acids 2-216 of SEQ ID NO: 75, and wherein n is about 8.

87. The ADC of claim 86, wherein the Ab comprises a HC consisting of SEQ ID NO: 74 and a LC consisting of SEQ ID NO: 75.

88. A pharmaceutical composition comprising the antibody of claim 1 and one or more pharmaceutically acceptable carriers, diluents, or excipients.

89. A pharmaceutical composition comprising the ADC of claim 35 and one or more pharmaceutically acceptable carriers, diluents, or excipients.

90. A method of treating cancer, comprising administering to a patient in need thereof, an effective amount of the ADC of claim 35.

91. The method of claim 90, wherein the cancer is urothelial carcinoma, breast cancer, lung cancer, gastric cancer, colorectal cancer, pancreatic cancer, head and neck cancer, ovarian cancer, or prostate cancer.

92. The method of claim 91, wherein the cancer is urothelial carcinoma.

93. The method of claim 90, wherein the patient being treated has relapsed after being administered enfortumab vedotin, or has become refractory to enfortumab vedotin.

94. The method of claim 90, wherein the patient is ineligible for treatment with enfortumab vedotin.

95. The method of claim 90, further comprising administrating simultaneously, separately, or sequentially a PD-1 inhibitor or PD-L1 inhibitor.

96. A method of producing an ADC, the method comprising contacting the antibody of claim 1 with a compound of the formula:

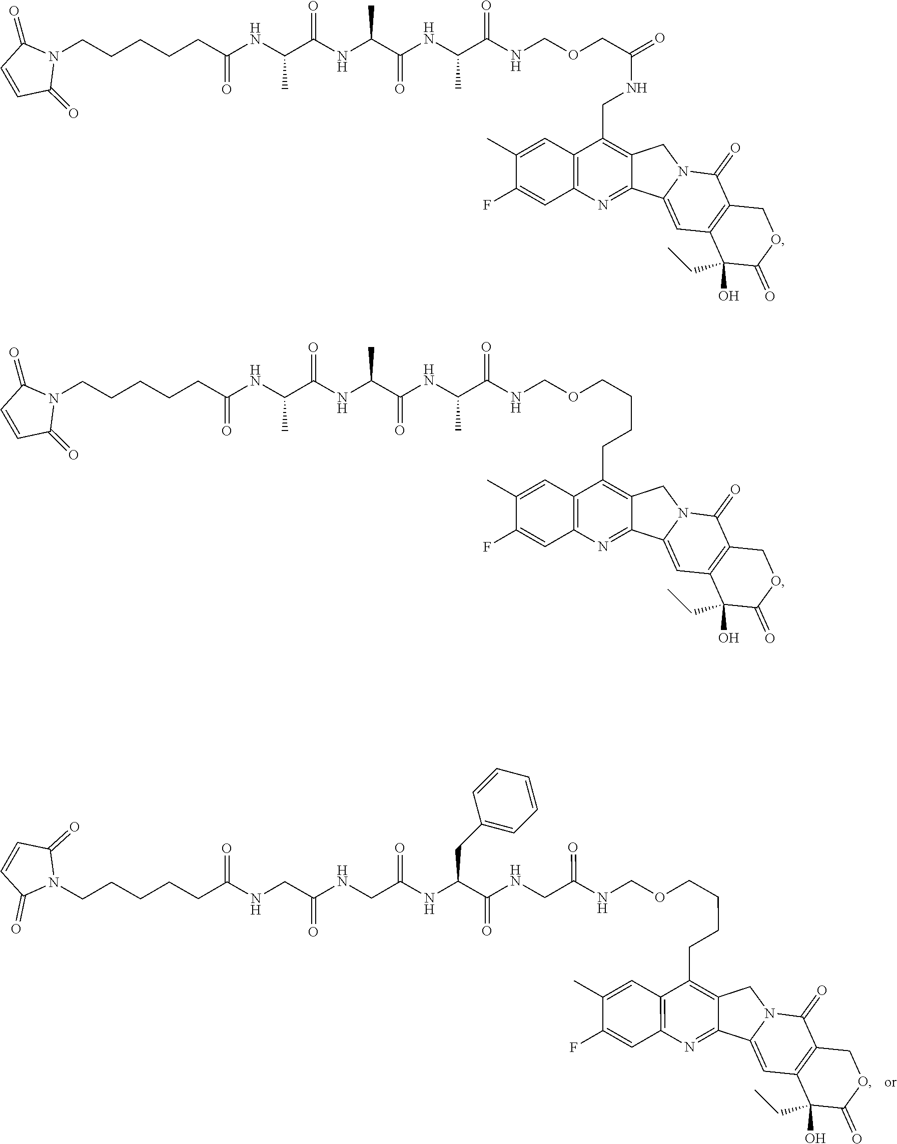 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)CCCCCN1C(=O)C=CC1=O]

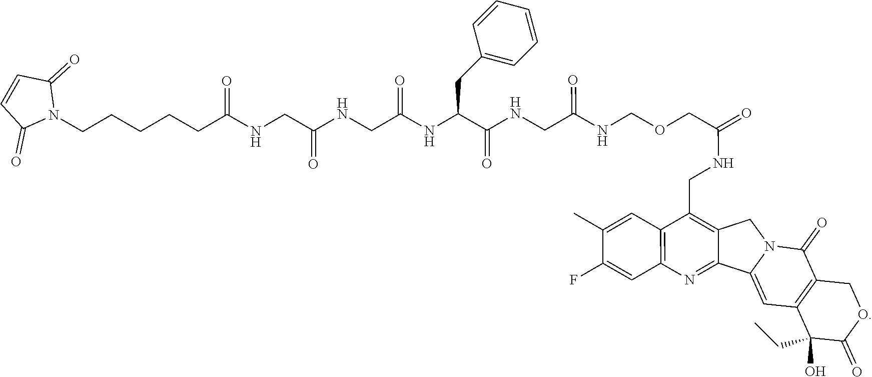 [SMILES: CC[C@@]1(O)C(=O)OCc2c1cc1n(c2=O)Cc2c-1nc1cc(F)c(C)cc1c2CNC(=O)COCNC(=O)CNC(=O)[C@H](Cc1ccccc1)NC(=O)CNC(=O)CNC(=O)CCCCCN1C(=O)C=CC1=O]

97. The method of claim 96, further comprising, reducing the antibody with a reducing agent to produce a reduced nectin-4 antibody prior to the contacting.

98. The method of claim 97, wherein the reducing agent is DTT or TCEP.


SEQUENCE LISTING (110 sequences)

SEQ ID NO: 1 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 2 - protein, 446 aa, synthetic construct
QVQLVQSGAEVKKPGASVKVSCKASGYTFTGYYIHWVRQAPGQGLEWMGWINPNSGGTNYAQKFQGRVTMTRDTSISTAYMELSRLRSDDTAVYYCAREDWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 215 aa, synthetic construct
EIVLTQSPGTLSLSPGERATLSCRTSQSVSSSYLAWYQQKPGQAPRLLIYGASNRATGIPDRFSGSGSGTDFTLTISRLEPEDFAVYYCQQYGSSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 13 aa, synthetic construct
KASGYTFTGYYIH

SEQ ID NO: 5 - protein, 10 aa, synthetic construct
WINPNSGGTN

SEQ ID NO: 6 - protein, 9 aa, synthetic construct
AREDWDFDY

SEQ ID NO: 7 - protein, 12 aa, synthetic construct
RTSQSVSSSYLA

SEQ ID NO: 8 - protein, 8 aa, synthetic construct
YGASNRAT

SEQ ID NO: 9 - protein, 9 aa, synthetic construct
QQYGSSPIT

SEQ ID NO: 10 - protein, 116 aa, synthetic construct
QVQLVQSGAEVKKPGASVKVSCKASGYTFTGYYIHWVRQAPGQGLEWMGWINPNSGGTNYAQKFQGRVTMTRDTSISTAYMELSRLRSDDTAVYYCAREDWDFDYWGQGTLVTVSS

SEQ ID NO: 11 - protein, 108 aa, synthetic construct
EIVLTQSPGTLSLSPGERATLSCRTSQSVSSSYLAWYQQKPGQAPRLLIYGASNRATGIPDRFSGSGSGTDFTLTISRLEPEDFAVYYCQQYGSSPITFGQGTKVEIK

SEQ ID NO: 12 - protein, 446 aa, synthetic construct
QVQLVQSGAQVKKPGASVKVSCKASGYTFTGYYIHWVRQAPGQGLEWMGWINPNSGGTNYAQKFQGRVTMTRDTSISTAYMELSRLRSDDTAVYFCAREDWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 215 aa, synthetic construct
EIVLTQSPGTLSLSPGERATLSCRTSQSVSSSYLAWYQQKPGQAPRLLIYGASNRATDIPDRFSGSGSGTDFTLTINRLEPEDFAVYYCQQYGSSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 116 aa, synthetic construct
QVQLVQSGAQVKKPGASVKVSCKASGYTFTGYYIHWVRQAPGQGLEWMGWINPNSGGTNYAQKFQGRVTMTRDTSISTAYMELSRLRSDDTAVYFCAREDWDFDYWGQGTLVTVSS

SEQ ID NO: 15 - protein, 108 aa, synthetic construct
EIVLTQSPGTLSLSPGERATLSCRTSQSVSSSYLAWYQQKPGQAPRLLIYGASNRATDIPDRFSGSGSGTDFTLTINRLEPEDFAVYYCQQYGSSPITFGQGTRLEIK

SEQ ID NO: 16 - protein, 445 aa, synthetic construct
EVQLVESGGGLVQPGGSLRLSCAASGFTFSSYDMHWVRQATGKGLEWVSAIGTVGDTYYPGSVKGRFTISRENAKNSLYLQMNSLRAGDTAVYYCAREWNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 220 aa, synthetic construct
DIVMTQSPDSLAVSLGERATINCKSSQSVLYNSNNKNYLAWYQQKPGQPPKLLIYWASTRESGVPDRFSGSGSGTDFTLTISSLQAEDVAVYYCQQFYTTPYSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 13 aa, synthetic construct
AASGFTFSSYDMH

SEQ ID NO: 19 - protein, 9 aa, synthetic construct
AIGTVGDTY

SEQ ID NO: 20 - protein, 9 aa, synthetic construct
AREWNGMDV

SEQ ID NO: 21 - protein, 17 aa, synthetic construct
KSSQSVLYNSNNKNYLA

SEQ ID NO: 22 - protein, 8 aa, synthetic construct
YWASTRES

SEQ ID NO: 23 - protein, 9 aa, synthetic construct
QQFYTTPYS

SEQ ID NO: 24 - protein, 115 aa, synthetic construct
EVQLVESGGGLVQPGGSLRLSCAASGFTFSSYDMHWVRQATGKGLEWVSAIGTVGDTYYPGSVKGRFTISRENAKNSLYLQMNSLRAGDTAVYYCAREWNGMDVWGQGTTVTVSS

SEQ ID NO: 25 - protein, 113 aa, synthetic construct
DIVMTQSPDSLAVSLGERATINCKSSQSVLYNSNNKNYLAWYQQKPGQPPKLLIYWASTRESGVPDRFSGSGSGTDFTLTISSLQAEDVAVYYCQQFYTTPYSFGQGTKVEIK

SEQ ID NO: 26 - protein, 449 aa, synthetic construct
EVQLVESGGGLVKPGGSLRLSCAASGFNLNHYNMNWVRQAPGKGLEWVSSVSSGGGFRYYADSVRGRFTISRDNAKNSLYLQMNSLRAEDTAVYYCARGAVFHDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, synthetic construct
DIQLTQSPSFLSASVGDRVTITCRASQDISSYLAWYQQKPGKAPKLLIYVASTLQSGVPSRFSGSGSGTEFTLTISSLQPEDFATYYCQQINSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 13 aa, synthetic construct
AASGFNLNHYNMN

SEQ ID NO: 29 - protein, 10 aa, synthetic construct
SVSSGGGFRY

SEQ ID NO: 30 - protein, 12 aa, synthetic construct
ARGAVFHDAFDI

SEQ ID NO: 31 - protein, 11 aa, synthetic construct
RASQDISSYLA

SEQ ID NO: 32 - protein, 8 aa, synthetic construct
YVASTLQS

SEQ ID NO: 33 - protein, 9 aa, synthetic construct
QQINSYPFT

SEQ ID NO: 34 - protein, 119 aa, synthetic construct
EVQLVESGGGLVKPGGSLRLSCAASGFNLNHYNMNWVRQAPGKGLEWVSSVSSGGGFRYYADSVRGRFTISRDNAKNSLYLQMNSLRAEDTAVYYCARGAVFHDAFDIWGQGTLVTVSS

SEQ ID NO: 35 - protein, 107 aa, synthetic construct
DIQLTQSPSFLSASVGDRVTITCRASQDISSYLAWYQQKPGKAPKLLIYVASTLQSGVPSRFSGSGSGTEFTLTISSLQPEDFATYYCQQINSYPFTFGQGTKVEIK

SEQ ID NO: 36 - protein, 448 aa, synthetic construct
QVQLQESGPGLVKPSETLSLTCTVSGGSISGYYWSWIRQPPGKGLEWIGYIYYSGSTNYNPSLKSRVTISVDTSKNQFSLKLSSVTAADTAVYYCARLGIFFDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQFNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 13 aa, synthetic construct
TVSGGSISGYYWS

SEQ ID NO: 39 - protein, 9 aa, synthetic construct
YIYYSGSTN

SEQ ID NO: 40 - protein, 12 aa, synthetic construct
ARLGIFFDAFDI

SEQ ID NO: 41 - protein, 11 aa, synthetic construct
RASQGISSYLA

SEQ ID NO: 42 - protein, 8 aa, synthetic construct
YAASTLQS

SEQ ID NO: 43 - protein, 9 aa, synthetic construct
QQFNSYPWT

SEQ ID NO: 44 - protein, 118 aa, synthetic construct
QVQLQESGPGLVKPSETLSLTCTVSGGSISGYYWSWIRQPPGKGLEWIGYIYYSGSTNYNPSLKSRVTISVDTSKNQFSLKLSSVTAADTAVYYCARLGIFFDAFDIWGQGTLVTVSS

SEQ ID NO: 45 - protein, 107 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQFNSYPWTFGQGTKVEIK

SEQ ID NO: 46 - protein, 448 aa, synthetic construct
QVQLVQSGAEVKKPGASVKVSCKASGYTFTSYYVHWVRQAPGQGLEWMGIINPSIISTSYAQKFQARVTMTRDTSTSTVYMELSSLRSEDTAVYYCARGLNFDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 214 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQLNNYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 13 aa, synthetic construct
KASGYTFTSYYVH

SEQ ID NO: 49 - protein, 10 aa, synthetic construct
IINPSIISTS

SEQ ID NO: 50 - protein, 11 aa, synthetic construct
ARGLNFDAFDI

SEQ ID NO: 51 - protein, 9 aa, synthetic construct
QQLNNYPFT

SEQ ID NO: 52 - protein, 118 aa, synthetic construct
QVQLVQSGAEVKKPGASVKVSCKASGYTFTSYYVHWVRQAPGQGLEWMGIINPSIISTSYAQKFQARVTMTRDTSTSTVYMELSSLRSEDTAVYYCARGLNFDAFDIWGQGTLVTVSS

SEQ ID NO: 53 - protein, 107 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQLNNYPFTFGQGTKVEIK

SEQ ID NO: 54 - protein, 448 aa, synthetic construct
QVQLVQSGAEVKKPGASVKVSCKASGYTFTGYYMHWVRQAPGQGLEWMGIINPSGGSTTYAQKFQGRVTMTRDTSTSTVYMELSSLRSEDTAVYYCARGRLGTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 214 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDRSSDQVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 13 aa, synthetic construct
KASGYTFTGYYMH

SEQ ID NO: 57 - protein, 10 aa, synthetic construct
IINPSGGSTT

SEQ ID NO: 58 - protein, 11 aa, synthetic construct
ARGRLGTYFDY

SEQ ID NO: 59 - protein, 11 aa, synthetic construct
GGNNIGSKSVH

SEQ ID NO: 60 - protein, 8 aa, synthetic construct
YDDSDRPS

SEQ ID NO: 61 - protein, 11 aa, synthetic construct
QVWDRSSDQVV

SEQ ID NO: 62 - protein, 118 aa, synthetic construct
QVQLVQSGAEVKKPGASVKVSCKASGYTFTGYYMHWVRQAPGQGLEWMGIINPSGGSTTYAQKFQGRVTMTRDTSTSTVYMELSSLRSEDTAVYYCARGRLGTYFDYWGQGTLVTVSS

SEQ ID NO: 63 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDRSSDQVVFGGGTKLTVL

SEQ ID NO: 64 - protein, 452 aa, synthetic construct
QVQLVQSGAEVKKPGASVKVSCKASGYTFTNYYMHWVRQAPGQGLEWMGIINPSSGSASYAQKFQGRVTMTRDTSTSTVYMELSSLRSEDTAVYYCAREGVGRDILQAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 216 aa, synthetic construct
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNGHVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 13 aa, synthetic construct
KASGYTFTNYYMH

SEQ ID NO: 67 - protein, 10 aa, synthetic construct
IINPSSGSAS

SEQ ID NO: 68 - protein, 15 aa, synthetic construct
AREGVGRDILQAFDI

SEQ ID NO: 69 - protein, 13 aa, synthetic construct
SGSSSNIGNNAVN

SEQ ID NO: 70 - protein, 8 aa, synthetic construct
YYDDLLPS

SEQ ID NO: 71 - protein, 11 aa, synthetic construct
AAWDDSLNGHV

SEQ ID NO: 72 - protein, 122 aa, synthetic construct
QVQLVQSGAEVKKPGASVKVSCKASGYTFTNYYMHWVRQAPGQGLEWMGIINPSSGSASYAQKFQGRVTMTRDTSTSTVYMELSSLRSEDTAVYYCAREGVGRDILQAFDIWGQGTLVTVSS

SEQ ID NO: 73 - protein, 110 aa, synthetic construct
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNGHVFGGGTKLTVL

SEQ ID NO: 74 - protein, 452 aa, synthetic construct
QVQLVQSGAEVKKPGASVKVSCKASGYTFTSYYIHWVRQAPGQGLEWMGIINPISGRTSSAQKFQGRVTMTRDTSTSTVYMELSSLRSEDTAVYYCAKEGVGGELLRAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 216 aa, synthetic construct
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNGFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 76 - protein, 13 aa, synthetic construct
KASGYTFTSYYIH

SEQ ID NO: 77 - protein, 10 aa, synthetic construct
IINPISGRTS

SEQ ID NO: 78 - protein, 15 aa, synthetic construct
AKEGVGGELLRAFDI

SEQ ID NO: 79 - protein, 11 aa, synthetic construct
AAWDDSLNGFV

SEQ ID NO: 80 - protein, 122 aa, synthetic construct
QVQLVQSGAEVKKPGASVKVSCKASGYTFTSYYIHWVRQAPGQGLEWMGIINPISGRTSSAQKFQGRVTMTRDTSTSTVYMELSSLRSEDTAVYYCAKEGVGGELLRAFDIWGQGTLVTVSS

SEQ ID NO: 81 - protein, 110 aa, synthetic construct
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNGFVFGGGTKLTVL

SEQ ID NO: 82 - DNA, 1338 bp, synthetic construct
  source 1..1338: mol_type = other DNA
caagttcagctggtgcagtctggagccgaagtgaagaagcccggagcctcagtgaaagtgtcctgtaaggcgtccggctataccttcactggttactatatccactgggtacgccaggctccaggacagggcttggagtggatgggttggatcaatcctaactctgggggcaccaactacgcacagaaattccaagggagagtcaccatgacgcgggatactagcattagcacagcttatatggagctctcgcgtctgaggagtgacgatactgccgtctactactgcgcacgagaagactgggactttgattattgggggcagggcacacttgtgacagtttcaagtgcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 83 - DNA, 645 bp, synthetic construct
  source 1..645: mol_type = other DNA
gagatcgtgctgacccagagtccaggaacactcagcctgtcccccggagaacgggctactttgtcatgccgtacgagccagagcgtgtcctcttcttatctggcttggtaccagcaaaagcctggacaagcgcctcgattacttatctatggtgcctctaaccgcgccacaggcattccagacagattctcagggtctggcagtggcaccgactttacactaaccatttccaggctcgaacccgaggatttcgcagtctactactgtcagcagtatgggtcgagcccgattacttttggccagggtaccaaggttgagatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 84 - DNA, 1338 bp, synthetic construct
  source 1..1338: mol_type = other DNA
caggttcaacttgtgcaaagtggagcacaggttaagaagcctggcgcatccgtgaaagtctcttgcaaagcatccgggtacacattcaccggctactatatccattgggtacggcaggccccaggtcaggggcttgaatggatgggatggataaatcccaatagcggtggtaccaattacgcccagaagtttcagggacgagtcacaatgactagagatacaagtatctcaaccgcatacatggaactctcaagactgagatcagatgacaccgcagtctatttctgtgctcgcgaggattgggacttcgattactggggccaaggcaccctggttaccgtcagcagcgcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 85 - DNA, 645 bp, synthetic construct
  source 1..645: mol_type = other DNA
gaaatcgtgcttacccaatcccctggtactctctccctcagccccggcgaacgggccaccctctcctgccgaacaagccaaagtgtgtctagctcctacctcgcctggtatcaacagaaacccggccaagcaccacgacttcttatctatggtgcaagcaacagggctactgacattccagatcgcttcagtggctctggctcaggaacagacttcactctgactatcaacaggttggaacctgaggattttgccgtatattattgccaacaatacggctcctctcctataacctttggtcaaggtacacgcctggaaataaagagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 86 - DNA, 1335 bp, synthetic construct
  source 1..1335: mol_type = other DNA
gaagtccaactagtggagagcggcgggggcctggtccagcccgggggttctctccgtctctcttgcgctgcatccggattcacattctcatcctatgatatgcactgggtgcgacaggcaacggggaagggcctggagtgggttagcgccattggtactgtgggcgacacttactatcctggaagcgtgaagggacggtttacaatctcaagggagaatgccaaaaacagtctgtaccttcagatgaacagtttgcgcgctggcgacaccgccgtttactattgtgcgagagaatggaatggaatggatgtgtggggccagggtaccaccgtcacagtatcctctgcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 87 - DNA, 660 bp, synthetic construct
  source 1..660: mol_type = other DNA
gacattgtcatgacccagtcacctgactccttggcggtgagcctgggagaaagagcaacaattaactgcaaatccagtcaatcggtcctctacaactccaataacaagaattacctggcttggtatcagcagaagcccggccagccacctaagctcctgatctattgggctagtactcgggagtccggagttcccgataggttctctggttcagggtctggcaccgactttacccttacaataagcagcctacaggccgaagatgtagccgtgtattactgtcaacagttctatacaacgccatactcttttggccaggggactaaagtggagatcaagagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 88 - DNA, 1347 bp, synthetic construct
  source 1..1347: mol_type = other DNA
gaggtgcagctggttgagagcggcggcgggttagtcaagcctggtggctcccttcggctgtcctgtgcagcctcggggttcaatctgaaccactacaacatgaactgggtgcgtcaagcccccggcaagggactcgaatgggtctctagtgtttcctcagggggtggatttcgctactatgctgacagcgtaaggggacgatttaccatttctcgggataatgccaaaaacagcttgtacctgcagatgaatagtctaagagcagaagataccgctgtgtattattgcgcgagaggtgccgtcttccatgacgctttcgacatctggggacagggcactctcgtgacagtgtcatctgcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 89 - DNA, 642 bp, synthetic construct
  source 1..642: mol_type = other DNA
gatatccaactgacccagtctccaagtttcctgtctgcttcagtgggcgatagggtcacaatcacctgtcgggcctcccaagacatctcctcgtacctggcgtggtaccagcagaagcctggcaaggctcccaaattgctcatatacgttgcaagtacccttcagagcggagtgccatccagattctcagggtccggaagcgggactgaatttacactaacgatcagcagcctccagcccgaggacttcgccacctattattgccagcagattaactcttatccttttacatttggccagggtactaaagtagagattaagagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 90 - DNA, 1344 bp, synthetic construct
  source 1..1344: mol_type = other DNA
caagtgcagctgcaggagagtggaccaggtctcgtgaagcctagtgagacactttcactcacttgtacagtaagtggcggttctatcagcgggtattactggtcctggattcggcagcctcccgggaagggattagaatggatcggatatatctattattccggcagcaccaattacaacccctctctaaaatccagggttacgataagcgtggatacctctaaaaaccagttttcgttgaagctgtctagcgtcaccgcagctgacaccgccgtgtactactgcgctagactgggcatcttctttgacgccttcgatatttgggggcagggcactctggtcacagtttcctcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 91 - DNA, 642 bp, synthetic construct
  source 1..642: mol_type = other DNA
gatatacaactaacacagtctccttccttcttgtctgctagtgtgggcgacagggtcacaatcacttgccgggcaagccagggaatttcctcctacctggcatggtatcagcagaagcccgggaaagctcccaagctcctgatctatgccgccagcacgcttcagagcggggtgccatccagattttcgggtagcggctctggcactgagtttaccctcacaattagttcactgcagcctgaagactttgccacctactactgtcaacagttcaactcatatccatggaccttcggtcagggaaccaaagttgagatcaagagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 92 - DNA, 1344 bp, synthetic construct
  source 1..1344: mol_type = other DNA
caggtccagctggttcagtccggagcagaggtaaaaaaaccaggagctagtgtcaaggtgtcctgtaaggcgtctgggtacacttttacttcatactatgttcactgggtgcgacaggcccctgggcagggcctcgaatggatgggcatcataaaccccagtatcatttccaccagctacgctcaaaagtttcaagcccgggtgaccatgacgagggatacctcgacatcaactgtctatatggagctatcctctttgagatctgaagatacagccgtgtattactgcgcacgcggccttaatttcgacgctttcgacatttggggtcagggtacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 93 - DNA, 642 bp, synthetic construct
  source 1..642: mol_type = other DNA
gacatccagttaactcagtctcctagcttcctgagcgcttctgttggagatagagtcacaattacatgtagggcctcccaggggatttcatcatatctggcttggtatcaacagaagcctggcaaagcaccaaagctcctgatctatgccgcatctaccctacaatcgggtgtgccctcccggttcagtggctccgggagtggaacggaatttacccttaccatctccagcttgcagcccgaggacttcgccacatactactgccagcagctcaataactacccatttacttttggtcagggcaccaaagtggagataaagagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 94 - DNA, 1344 bp, synthetic construct
  source 1..1344: mol_type = other DNA
caagttcagctggtgcaatccggagctgaggtcaagaagcctggtgcaagcgtgaaagtgagttgtaaagcctcaggctataccttcactgggtattacatgcactgggttcggcaggctccaggacagggattggaatggatgggcatcattaaccccagcgggggttccacaacgtacgcccagaagtttcagggccgtgtgaccatgacacgcgatacttctacatctaccgtatacatggaactcagttccctgcgatctgaggacactgcagtctattactgcgccaggggcagacttggcacctatttcgactactggggtcaggggaccctagtcacagtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 95 - DNA, 642 bp, synthetic construct
  source 1..642: mol_type = other DNA
agctacgtccttactcagccacctagtgtgtcagtagcgcctgggcaaaccgctcgcataacttgcgggggaaacaacatcggctctaaatccgtccactggtaccagcagaagcccggccaggcacccgtattggttgtttacgacgatagcgacagaccgtccggtatcccagagcggttttccggtagtaattctggaaatacagctaccctgacaatttctagggtggaagccggggatgaggccgactattattgtcaagtgtgggatcgatcaagcgaccaggtggtcttcggcggaggcacgaagctgaccgtgctcggccagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 96 - DNA, 1356 bp, synthetic construct
  source 1..1356: mol_type = other DNA
caggtgcaactggtgcagagtggagccgaggtgaagaagcccggcgcttcggtcaaggtttcatgcaaagcatccgggtataccttcactaactactatatgcactgggttcgccaggccccaggtcaaggcttggaatggatgggaatcataaatccttctagcggttctgcatcctacgctcagaaatttcagggacgtgtcaccatgacaagagatacatctacttcaaccgtctacatggagctaagttccctgaggtccgaagacaccgccgtatattactgtgcgcgggagggcgtgggccgagacattctccaggctttcgatatctgggggcaggggacgcttgtgacagtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 97 - DNA, 648 bp, synthetic construct
  source 1..648: mol_type = other DNA
cagtctgttctcacacaaccaccctcagtaagcgaggctccccggcagagagtcacaatctcctgtagcggaagtagttcaaacatagggaataatgcggtgaactggtatcagcagttgcccggtaaagcccctaaactgctcatttactacgacgacttactaccttccggcgtgagcgacaggttttcaggaagcaagtctggcacttcggcctccctggcaatctctggacttcagagtgaggatgaagctgactattactgcgcagcctgggatgattccttgaacggccacgtcttcggtggcgggaccaagttgaccgtgctgggccagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 98 - DNA, 1356 bp, synthetic construct
  source 1..1356: mol_type = other DNA
caggtgcagctcgtccagagtggggccgaggtgaaaaagcctggcgcttcggtcaaggtttcatgcaaagcctccggttacaccttcacatcctattatatccactgggtgagacaagctccaggacagggcctggaatggatgggaattataaaccccattagcggtcgaacctcttctgcccagaagttccagggacgtgtgactatgactcgggatacatccacaagcaccgtatacatggagctatcttcacttaggagtgaggacactgcagtttactattgtgcgaaggaaggtgtcgggggcgagctgttgcgcgcatttgacatctgggggcaaggcaccctggtgacggtgtccagcgcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 99 - DNA, 648 bp, synthetic construct
  source 1..648: mol_type = other DNA
cagagtgtgctgacccagcccccaagcgtctccgaggcacccagacagagggtgacaatctcctgctccggaagcagttcaaacataggcaacaacgccgtaaattggtaccagcagctgccaggtaaggcccctaaacttctgatctattacgatgatttgctcccttcaggtgtctctgaccgcttttccggaagcaaatctgggaccagcgcttctttggcaatttctggcctgcaatcggaagacgaggctgattactattgtgccgcttgggacgacagtttaaatgggttcgttttcggcggcggaacaaagctaactgtgctcggccagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 100 - protein, 4 aa, synthetic construct
ALAL

SEQ ID NO: 101 - protein, 4 aa, synthetic construct
LALA

SEQ ID NO: 102 - protein, 4 aa, synthetic construct
GGFG

SEQ ID NO: 103 - protein, 4 aa, synthetic construct
GFLG

SEQ ID NO: 104 - protein, 4 aa, synthetic construct
GLFG

SEQ ID NO: 105 - protein, 4 aa, synthetic construct
AAAA

SEQ ID NO: 106 - protein, 4 aa, synthetic construct
GAGG

SEQ ID NO: 107 - protein, 4 aa, synthetic construct
GGAG

SEQ ID NO: 108 - protein, 4 aa, synthetic construct
GVGG

SEQ ID NO: 109 - protein, 4 aa, synthetic construct
GGVG

SEQ ID NO: 110 - protein, 4 aa, synthetic construct
GFGG